SEQUENCE LISTING (191 sequences)

SEQ ID NO: 1 - protein, 126 aa, synthetic construct
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 2 - protein, 107 aa, synthetic construct
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 3 - protein, 5 aa, synthetic construct
GYYMH

SEQ ID NO: 4 - protein, 10 aa, synthetic construct
GYTFTGYYMH

SEQ ID NO: 5 - protein, 8 aa, synthetic construct
GYTFTGYY

SEQ ID NO: 6 - protein, 17 aa, synthetic construct
WINPDSGGTNYAQKFQG

SEQ ID NO: 7 - protein, 10 aa, synthetic construct
WINPDSGGTN

SEQ ID NO: 8 - protein, 8 aa, synthetic construct
WINPDSGG

SEQ ID NO: 9 - protein, 8 aa, synthetic construct
INPDSGGT

SEQ ID NO: 10 - protein, 17 aa, synthetic construct
DQPLGYCTNGVCSYFDY

SEQ ID NO: 11 - protein, 19 aa, synthetic construct
ARDQPLGYCTNGVCSYFDY

SEQ ID NO: 12 - protein, 11 aa, synthetic construct
RASQGIYSWLA

SEQ ID NO: 13 - protein, 6 aa, synthetic construct
QGIYSW

SEQ ID NO: 14 - protein, 7 aa, synthetic construct
TASTLQS

SEQ ID NO: 15 - skipped

SEQ ID NO: 16 - protein, 9 aa, synthetic construct
QQANIFPLT

SEQ ID NO: 17 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTRFVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 18 - protein, 107 aa, synthetic construct [sequence omitted]

SEQ ID NO: 19 - protein, 452 aa, synthetic construct
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 20 - protein, 473 aa, synthetic construct
DVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSSGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 473 aa, synthetic construct
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSS

SEQ ID NO: 22 - protein, 711 aa, synthetic construct
DVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSSGGGGSGGGGSGGGGSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 23 - protein, 214 aa, synthetic construct
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 711 aa, synthetic construct
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSDVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSS

SEQ ID NO: 25 - protein, 479 aa, synthetic construct
QVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSSGGGGSGGGGSGGGGSGGGGASDVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIKGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 479 aa, synthetic construct
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSSGGGGSGGGGSGGGGSGGGGASDVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIK

SEQ ID NO: 27 - protein, 717 aa, synthetic construct
QVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSSGGGGSGGGGSGGGGSGGGGASDVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 28 - protein, 717 aa, synthetic construct
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSQVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSSGGGGSGGGGSGGGGSGGGGASDVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIK

SEQ ID NO: 29 - protein, 473 aa, synthetic construct
DVVMTQSPDSLAVSLGERATINCKSSQSIVHRNGNTYLEWYQLKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTMHWVRQAPGQGLEWMGYINPSSGYAAYNQKFQGRVTMTADKSTSTAYMELSSLRSEDTAVYYCAREKFYGYDYWGQGTTVTVSSGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 473 aa, synthetic construct
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDVVMTQSPDSLAVSLGERATINCKSSQSIVHRNGNTYLEWYQLKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTMHWVRQAPGQGLEWMGYINPSSGYAAYNQKFQGRVTMTADKSTSTAYMELSSLRSEDTAVYYCAREKFYGYDYWGQGTTVTVSS

SEQ ID NO: 31 - protein, 711 aa, synthetic construct
DVVMTQSPDSLAVSLGERATINCKSSQSIVHRNGNTYLEWYQLKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTMHWVRQAPGQGLEWMGYINPSSGYAAYNQKFQGRVTMTADKSTSTAYMELSSLRSEDTAVYYCAREKFYGYDYWGQGTTVTVSSGGGGSGGGGSGGGGSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 32 - protein, 711 aa, synthetic construct
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSDVVMTQSPDSLAVSLGERATINCKSSQSIVHRNGNTYLEWYQLKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTMHWVRQAPGQGLEWMGYINPSSGYAAYNQKFQGRVTMTADKSTSTAYMELSSLRSEDTAVYYCAREKFYGYDYWGQGTTVTVSS

SEQ ID NO: 33 - protein, 116 aa, synthetic construct
EVQLVQSGAEVKKPGESLRISCKGSGDSFTTYWIGWVRQMPGKGLEWMGIIYPGDSDTIYSPSFQGQVTISADKSISTAYLQWSSLKASDTAMYYCTRGDRGVDYWGQGTLVTVSS

SEQ ID NO: 34 - protein, 107 aa, synthetic construct
DIQMTQSPSSLSASVGDRVTITCRASQGISRWLAWYQQKPEKAPKSLIYAASSLQSGVPSRFSGSGSGTDFTLTISGLQPEDFATYYCQQYNSYPRTFGQGTKVEIK

SEQ ID NO: 35 - protein, 5 aa, synthetic construct
TYWIG

SEQ ID NO: 36 - protein, 10 aa, synthetic construct
GDSFTTYWIG

SEQ ID NO: 37 - protein, 8 aa, synthetic construct
GDSFTTYW

SEQ ID NO: 38 - protein, 17 aa, synthetic construct
IIYPGDSDTIYSPSFQG

SEQ ID NO: 39 - protein, 10 aa, synthetic construct
IIYPGDSDTI

SEQ ID NO: 40 - protein, 7 aa, synthetic construct
IIYPGDS

SEQ ID NO: 41 - protein, 8 aa, synthetic construct
IYPGDSDT

SEQ ID NO: 42 - protein, 7 aa, synthetic construct
GDRGVDY

SEQ ID NO: 43 - protein, 9 aa, synthetic construct
TRGDRGVDY

SEQ ID NO: 44 - protein, 11 aa, synthetic construct
RASQGISRWLA

SEQ ID NO: 45 - protein, 6 aa, synthetic construct
QGISRW

SEQ ID NO: 46 - protein, 7 aa, synthetic construct
AASSLQS

SEQ ID NO: 47 - skipped

SEQ ID NO: 48 - protein, 9 aa, synthetic construct
QQYNSYPRT

SEQ ID NO: 49 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 446 aa, synthetic construct
EVQLVQSGAEVKKPGESLRISCKGSGDSFTTYWIGWVRQMPGKGLEWMGIIYPGDSDTIYSPSFQGQVTISADKSISTAYLQWSSLKASDTAMYYCTRGDRGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 473 aa, synthetic construct
DVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSSGGGGSGGGGSGGGGSDIQMTQSPSSLSASVGDRVTITCRASQGISRWLAWYQQKPEKAPKSLIYAASSLQSGVPSRFSGSGSGTDFTLTISGLQPEDFATYYCQQYNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 473 aa, synthetic construct
DIQMTQSPSSLSASVGDRVTITCRASQGISRWLAWYQQKPEKAPKSLIYAASSLQSGVPSRFSGSGSGTDFTLTISGLQPEDFATYYCQQYNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSS

SEQ ID NO: 53 - protein, 705 aa, synthetic construct
DVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSSGGGGSGGGGSGGGGSEVQLVQSGAEVKKPGESLRISCKGSGDSFTTYWIGWVRQMPGKGLEWMGIIYPGDSDTIYSPSFQGQVTISADKSISTAYLQWSSLKASDTAMYYCTRGDRGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 214 aa, synthetic construct
DIQMTQSPSSLSASVGDRVTITCRASQGISRWLAWYQQKPEKAPKSLIYAASSLQSGVPSRFSGSGSGTDFTLTISGLQPEDFATYYCQQYNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 705 aa, synthetic construct
EVQLVQSGAEVKKPGESLRISCKGSGDSFTTYWIGWVRQMPGKGLEWMGIIYPGDSDTIYSPSFQGQVTISADKSISTAYLQWSSLKASDTAMYYCTRGDRGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSS

SEQ ID NO: 56 - protein, 479 aa, synthetic construct
QVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSSGGGGSGGGGSGGGGSGGGGASDVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIKGGGGSGGGGSGGGGSDIQMTQSPSSLSASVGDRVTITCRASQGISRWLAWYQQKPEKAPKSLIYAASSLQSGVPSRFSGSGSGTDFTLTISGLQPEDFATYYCQQYNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 479 aa, synthetic construct
DIQMTQSPSSLSASVGDRVTITCRASQGISRWLAWYQQKPEKAPKSLIYAASSLQSGVPSRFSGSGSGTDFTLTISGLQPEDFATYYCQQYNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSSGGGGSGGGGSGGGGSGGGGASDVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIK

SEQ ID NO: 58 - protein, 711 aa, synthetic construct
QVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSSGGGGSGGGGSGGGGSGGGGASDVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIKGGGGSGGGGSGGGGSEVQLVQSGAEVKKPGESLRISCKGSGDSFTTYWIGWVRQMPGKGLEWMGIIYPGDSDTIYSPSFQGQVTISADKSISTAYLQWSSLKASDTAMYYCTRGDRGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 711 aa, synthetic construct
EVQLVQSGAEVKKPGESLRISCKGSGDSFTTYWIGWVRQMPGKGLEWMGIIYPGDSDTIYSPSFQGQVTISADKSISTAYLQWSSLKASDTAMYYCTRGDRGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSSGGGGSGGGGSGGGGSGGGGASDVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIK

SEQ ID NO: 60 - protein, 473 aa, synthetic construct
DVVMTQSPDSLAVSLGERATINCKSSQSIVHRNGNTYLEWYQLKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTMHWVRQAPGQGLEWMGYINPSSGYAAYNQKFQGRVTMTADKSTSTAYMELSSLRSEDTAVYYCAREKFYGYDYWGQGTTVTVSSGGGGSGGGGSGGGGSDIQMTQSPSSLSASVGDRVTITCRASQGISRWLAWYQQKPEKAPKSLIYAASSLQSGVPSRFSGSGSGTDFTLTISGLQPEDFATYYCQQYNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 473 aa, synthetic construct
DIQMTQSPSSLSASVGDRVTITCRASQGISRWLAWYQQKPEKAPKSLIYAASSLQSGVPSRFSGSGSGTDFTLTISGLQPEDFATYYCQQYNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDVVMTQSPDSLAVSLGERATINCKSSQSIVHRNGNTYLEWYQLKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTMHWVRQAPGQGLEWMGYINPSSGYAAYNQKFQGRVTMTADKSTSTAYMELSSLRSEDTAVYYCAREKFYGYDYWGQGTTVTVSS

SEQ ID NO: 62 - protein, 705 aa, synthetic construct
DVVMTQSPDSLAVSLGERATINCKSSQSIVHRNGNTYLEWYQLKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTMHWVRQAPGQGLEWMGYINPSSGYAAYNQKFQGRVTMTADKSTSTAYMELSSLRSEDTAVYYCAREKFYGYDYWGQGTTVTVSSGGGGSGGGGSGGGGSEVQLVQSGAEVKKPGESLRISCKGSGDSFTTYWIGWVRQMPGKGLEWMGIIYPGDSDTIYSPSFQGQVTISADKSISTAYLQWSSLKASDTAMYYCTRGDRGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 705 aa, synthetic construct
EVQLVQSGAEVKKPGESLRISCKGSGDSFTTYWIGWVRQMPGKGLEWMGIIYPGDSDTIYSPSFQGQVTISADKSISTAYLQWSSLKASDTAMYYCTRGDRGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDVVMTQSPDSLAVSLGERATINCKSSQSIVHRNGNTYLEWYQLKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTMHWVRQAPGQGLEWMGYINPSSGYAAYNQKFQGRVTMTADKSTSTAYMELSSLRSEDTAVYYCAREKFYGYDYWGQGTTVTVSS

SEQ ID NO: 64 - protein, 15 aa, synthetic construct
GGGGSGGGGSGGGGS

SEQ ID NO: 65 - protein, 21 aa, synthetic construct
GGGGSGGGGSGGGGSGGGGAS

SEQ ID NO: 66 - protein, 19 aa, synthetic construct
MKHLWFFLLLVAAPRWVLS

SEQ ID NO: 67 - protein, 20 aa, synthetic construct
MVLQTQVFISLLLWISGAYG

SEQ ID NO: 68 - protein, 514 aa, synthetic construct
EVHLVESGGGLVQPGGSLRLSCAASGFTFSRYWMSWVRQAQGKGLEWVASIKQAGSEKTYVDSVKGRFTISRDNAKNSLSLQMNSLRAEDTAVYYCAREGAYYYDSASYYPYYYYYSMDVWGQGTTVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSQYTFGQGTKLEIKGGGGSDYKDDDDKLSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 514 aa, synthetic construct
EVHLVESGGGLVQPGGSLRLSCAASGFTFSRYWMSWVRQAQGKGLEWVASIKQAGSEKTYVDSVKGRFTISRDNAKNSLSLQMNSLRAEDTAVYYCAREGAYYYDSASYYPYYYYYSMDVWGQGTTVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSQYTFGQGTKLEIKGGGGSDYKDDDDKLSNSIMYFSHFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 117 aa, synthetic construct
QVQLQQSAGELARPGASVKMSCKASGYSFTTYTIHWVKQRPGQGLEWIGYINPSSGYAAYNQNFKDETTLTADKSSSTAYMQLSSLTSEDSAVYYCAREKFYGYDYWGQGTTLTVSS

SEQ ID NO: 71 - protein, 117 aa, synthetic construct
QVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSS

SEQ ID NO: 72 - protein, 117 aa, synthetic construct
QVQLVQSGAEVKKPGASVKVSCKASGYSFTTYTMHWVRQAPGQGLEWMGYINPSSGYAAYNQKFQGRVTMTADKSTSTAYMELSSLRSEDTAVYYCAREKFYGYDYWGQGTTVTVSS

SEQ ID NO: 73 - protein, 117 aa, synthetic construct
QVQLVQSGSELKKPGASVKVSCKASGYSFTTYTIHWVRQAPGQGLEWIGYINPSSGYAAYNQGFTGRFVLSADKSVSTAYLQISSLKAEDTAVYYCAREKFYGYDYWGQGTTVTVSS

SEQ ID NO: 74 - protein, 117 aa, synthetic construct
QVQLVQSGSELKKPGASVKVSCKASGYSFTTYTMNWVRQAPGQGLEWMGYINPSSGYAAYNQGFTGRFVFSADKSVSTAYLQISSLKAEDTAVYYCAREKFYGYDYWGQGTTVTVSS

SEQ ID NO: 75 - protein, 112 aa, synthetic construct
DVLMTQLPLSLPVSLGDQASISCRSSQSIVHRNGNTYLEWYLLKPGQSPKLLIYKVSNRFSGVPDRFSGSGSGTDFTLKISRVEAEDLGVYYCFQGSHVPYTFGGGTKLEIK

SEQ ID NO: 76 - protein, 112 aa, synthetic construct
DVVMTQSPLSLPVTLGQPASISCRSSQSIVHRNGNTYLEWFQLRPGQSPRLLIYKVSNRFSGVPDRFSGSGSGTDFTLKISRVEAEDVGVYYCFQGSHVPYTFGGGTKLEIK

SEQ ID NO: 77 - protein, 112 aa, synthetic construct
DVVMTQSPLSLPVTLGQPASISCRSSQSIVHRNGNTYLEWFQQRPGQSPRLLIYKVSNRFSGVPDRFSGSGSGTDFTLKISRVEAEDVGVYYCFQGSHVPYTFGGGTKLEIK

SEQ ID NO: 78 - protein, 112 aa, synthetic construct
DVVMTQSPDSLAVSLGERATINCKSSQSIVHRNGNTYLEWYQLKPGQPPKLLIYKVSNRFSGVPDRFSGSGSGTDFTLTISSLQAEDVAVYYCFQGSHVPYTFGGGTKLEIK

SEQ ID NO: 79 - protein, 112 aa, synthetic construct
DVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIK

SEQ ID NO: 80 - protein, 4 aa, synthetic construct
DYKD

SEQ ID NO: 81 - protein, 8 aa, synthetic construct
GYSFTTYT

SEQ ID NO: 82 - protein, 8 aa, synthetic construct
INPSSGYA

SEQ ID NO: 83 - protein, 10 aa, synthetic construct
AREKFYGYDY

SEQ ID NO: 84 - protein, 25 aa, synthetic construct [sequence omitted]

SEQ ID NO: 85 - protein, 17 aa, synthetic construct
IHWVRQAPGQGLEWIGY

SEQ ID NO: 86 - protein, 17 aa, synthetic construct
MHWVRQAPGQGLEWMGY

SEQ ID NO: 87 - protein, 38 aa, synthetic construct
AYNQKFQGRVTLTADKSTSTAYMELSSLRSEDTAVYYC

SEQ ID NO: 88 - protein, 38 aa, synthetic construct
AYNQKFQGRVTMTADKSTSTAYMELSSLRSEDTAVYYC

SEQ ID NO: 89 - protein, 11 aa, synthetic construct
WGQGTTVTVSS

SEQ ID NO: 90 - protein, 11 aa, synthetic construct
QSIVHRNGNTY

SEQ ID NO: 91 - skipped

SEQ ID NO: 92 - protein, 9 aa, synthetic construct
FQGSHVPYT

SEQ ID NO: 93 - protein, 26 aa, synthetic construct
DVVMTQSPDSLAVSLGERATINCKSS

SEQ ID NO: 94 - protein, 17 aa, synthetic construct
LEWYQQKPGQPPKLLIY

SEQ ID NO: 95 - protein, 17 aa, synthetic construct
LEWYQLKPGQPPKLLIY

SEQ ID NO: 96 - protein, 36 aa, synthetic construct [sequence omitted]

SEQ ID NO: 97 - protein, 10 aa, synthetic construct
FGGGTKLEIK

SEQ ID NO: 98 - protein, 10 aa, synthetic construct
GDYKDDDDKG

SEQ ID NO: 99 - protein, 8 aa, synthetic construct
DYKDDDDK

SEQ ID NO: 100 - protein, 9 aa, synthetic construct
MDYKDDDDK

SEQ ID NO: 101 - protein, 7 aa, synthetic construct
DFKDDDK

SEQ ID NO: 102 - protein, 8 aa, synthetic construct
DYKAFDNL

SEQ ID NO: 103 - protein, 7 aa, synthetic construct
DYKDHDG

SEQ ID NO: 104 - protein, 9 aa, synthetic construct
MDFKDDDDK

SEQ ID NO: 105 - protein, 9 aa, synthetic construct
MDYKAFDNL

SEQ ID NO: 106 - protein, 7 aa, synthetic construct
DYKDHDI

SEQ ID NO: 107 - protein, 5 aa, synthetic construct
DYKDH

SEQ ID NO: 108 - protein, 5 aa, synthetic construct
DYKDD

SEQ ID NO: 109 - protein, 6 aa, synthetic construct
DYKDHD

SEQ ID NO: 110 - protein, 6 aa, synthetic construct
DYKDDD

SEQ ID NO: 111 - protein, 5 aa, synthetic construct
TYTIH

SEQ ID NO: 112 - protein, 5 aa, synthetic construct
TYTMH

SEQ ID NO: 113 - protein, 17 aa, synthetic construct
YINPSSGYAAYNQKFQG

SEQ ID NO: 114 - protein, 8 aa, synthetic construct
EKFYGYDY

SEQ ID NO: 115 - protein, 29 aa, synthetic construct
QVQLVQSGAEVKKPGASVKVSCKASGYSF

SEQ ID NO: 116 - protein, 14 aa, synthetic construct
WVRQAPGQGLEWIG

SEQ ID NO: 117 - protein, 14 aa, synthetic construct
WVRQAPGQGLEWMG

SEQ ID NO: 118 - protein, 32 aa, synthetic construct
RVTLTADKSTSTAYMELSSLRSEDTAVYYCAR

SEQ ID NO: 119 - protein, 32 aa, synthetic construct
RVTMTADKSTSTAYMELSSLRSEDTAVYYCAR

SEQ ID NO: 120 - protein, 16 aa, synthetic construct
KSSQSIVHRNGNTYLE

SEQ ID NO: 121 - protein, 7 aa, synthetic construct
KVSNRFS

SEQ ID NO: 122 - protein, 23 aa, synthetic construct
DVVMTQSPDSLAVSLGERATINC

SEQ ID NO: 123 - protein, 15 aa, synthetic construct
WYQQKPGQPPKLLIY

SEQ ID NO: 124 - protein, 15 aa, synthetic construct
WYQLKPGQPPKLLIY

SEQ ID NO: 125 - protein, 32 aa, synthetic construct
GVPDRFSGSGSGTDFTLTISSLQAEDVAVYYC

SEQ ID NO: 126 - protein, 7 aa, synthetic construct
GYSFTTY

SEQ ID NO: 127 - protein, 6 aa, synthetic construct
NPSSGY

SEQ ID NO: 128 - protein, 19 aa, synthetic construct
TIHWVRQAPGQGLEWIGYI

SEQ ID NO: 129 - protein, 19 aa, synthetic construct
TMHWVRQAPGQGLEWMGYI

SEQ ID NO: 130 - protein, 41 aa, synthetic construct
AAYNQKFQGRVTLTADKSTSTAYMELSSLRSEDTAVYYCAR

SEQ ID NO: 131 - protein, 41 aa, synthetic construct
AAYNQKFQGRVTMTADKSTSTAYMELSSLRSEDTAVYYCAR

SEQ ID NO: 132 - protein, 17 aa, synthetic construct
YINPSSGYAAYNQNFKD

SEQ ID NO: 133 - protein, 16 aa, synthetic construct
RSSQSIVHRNGNTYLE

SEQ ID NO: 134 - protein, 25 aa, synthetic construct
QVQLQQSAGELARPGASVKMSCKAS

SEQ ID NO: 135 - protein, 17 aa, synthetic construct
IHWVKQRPGQGLEWIGY

SEQ ID NO: 136 - protein, 38 aa, synthetic construct
AYNQNFKDETTLTADKSSSTAYMQLSSLTSEDSAVYYC

SEQ ID NO: 137 - protein, 11 aa, synthetic construct
WGQGTTLTVSS

SEQ ID NO: 138 - protein, 26 aa, synthetic construct
DVLMTQLPLSLPVSLGDQASISCRSS

SEQ ID NO: 139 - protein, 17 aa, synthetic construct
LEWYLLKPGQSPKLLIY

SEQ ID NO: 140 - protein, 36 aa, synthetic construct
NRFSGVPDRFSGSGSGTDFTLKISRVEAEDLGVYYC

SEQ ID NO: 141 - protein, 30 aa, synthetic construct
QVQLQQSAGELARPGASVKMSCKASGYSFT

SEQ ID NO: 142 - protein, 14 aa, synthetic construct
WVKQRPGQGLEWIG

SEQ ID NO: 143 - protein, 32 aa, synthetic construct
ETTLTADKSSSTAYMQLSSLTSEDSAVYYCAR

SEQ ID NO: 144 - protein, 23 aa, synthetic construct
DVLMTQLPLSLPVSLGDQASISC

SEQ ID NO: 145 - protein, 15 aa, synthetic construct
WYLLKPGQSPKLLIY

SEQ ID NO: 146 - protein, 33 aa, synthetic construct
GVPDRFSGSGSGTDFTLKISRVEAEDLGVYYCF

SEQ ID NO: 147 - protein, 19 aa, synthetic construct
TIHWVKQRPGQGLEWIGYI

SEQ ID NO: 148 - protein, 41 aa, synthetic construct
AAYNQNFKDETTLTADKSSSTAYMQLSSLTSEDSAVYYCAR

SEQ ID NO: 149 - protein, 25 aa, synthetic construct
QVQLVQSGSELKKPGASVKVSCKAS

SEQ ID NO: 150 - protein, 17 aa, synthetic construct
MNWVRQAPGQGLEWMGY

SEQ ID NO: 151 - protein, 38 aa, synthetic construct
AYNQGFTGRFVLSADKSVSTAYLQISSLKAEDTAVYYC

SEQ ID NO: 152 - protein, 38 aa, synthetic construct
AYNQGFTGRFVFSADKSVSTAYLQISSLKAEDTAVYYC

SEQ ID NO: 153 - protein, 26 aa, synthetic construct
DVVMTQSPLSLPVTLGQPASISCRSS

SEQ ID NO: 154 - protein, 17 aa, synthetic construct
LEWFQQRPGQSPRLLIY

SEQ ID NO: 155 - protein, 36 aa, synthetic construct
NRFSGVPDRFSGSGSGTDFTLKISRVEAEDVGVYYC

SEQ ID NO: 156 - protein, 5 aa, synthetic construct
TYTMN

SEQ ID NO: 157 - protein, 17 aa, synthetic construct
YINPSSGYAAYNQGFTG

SEQ ID NO: 158 - protein, 30 aa, synthetic construct
QVQLVQSGSELKKPGASVKVSCKASGYSFT

SEQ ID NO: 159 - protein, 32 aa, synthetic construct
RFVLSADKSVSTAYLQISSLKAEDTAVYYCAR

SEQ ID NO: 160 - protein, 32 aa, synthetic construct
RFVFSADKSVSTAYLQISSLKAEDTAVYYCAR

SEQ ID NO: 161 - protein, 23 aa, synthetic construct
DVVMTQSPLSLPVTLGQPASISC

SEQ ID NO: 162 - protein, 15 aa, synthetic construct
WFQQRPGQSPRLLIY

SEQ ID NO: 163 - protein, 32 aa, synthetic construct
GVPDRFSGSGSGTDFTLKISRVEAEDVGVYYC

SEQ ID NO: 164 - protein, 25 aa, synthetic construct
QVQLVQSGSELKKPGASVKVSCKAS

SEQ ID NO: 165 - protein, 19 aa, synthetic construct
TMNWVRQAPGQGLEWMGYI

SEQ ID NO: 166 - protein, 41 aa, synthetic construct
AAYNQGFTGRFVLSADKSVSTAYLQISSLKAEDTAVYYCAR

SEQ ID NO: 167 - protein, 41 aa, synthetic construct
AAYNQGFTGRFVFSADKSVSTAYLQISSLKAEDTAVYYCAR

SEQ ID NO: 168 - protein, 23 aa, synthetic construct
DVVMTQSPLSLPVTLGQPASISC

SEQ ID NO: 169 - protein, 15 aa, synthetic construct
WFQQRPGQSPRLLIY

SEQ ID NO: 170 - protein, 32 aa, synthetic construct; identical to SEQ ID NO: 163

SEQ ID NO: 171 - protein, 471 aa, synthetic construct
MKHLWFFLLLVAAPRWVLSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPCREEMTKNQVSLWCLVKGFYPSDIAVEWESNGQPENNYKTTPPMLDSDGSFFLYSKLTVDKSRWQQGNVFSCSVMHEALHNHYTQKSLSLSPGK

SEQ ID NO: 172 - protein, 234 aa, synthetic construct
MVLQTQVFISLLLWISGAYGDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 506 aa, synthetic construct
MKHLWFFLLLVAAPRWVLSDVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSSGGGGSGGGGSGGGGSERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVCTLPPSREEMTKNQVSLSCAVKGFYPSDIAVEWESNGQPENNYKTTPPMLDSDGSFFLVSKLTVDKSRWQQGNVFSCSVMHEALHNHYTQKSLSLSPGK

SEQ ID NO: 174 - protein, 471 aa, synthetic construct
MKHLWFFLLLVAAPRWVLSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLYCLVKGFYPSDIAVEWESNGQPENNYKTTPPMLDSDGSFFLYSKLTVDKSRWQQGNVFSCSVMHEALHNHYTQKSLSLSPGK

SEQ ID NO: 175 - protein, 506 aa, synthetic construct
MKHLWFFLLLVAAPRWVLSDVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSSGGGGSGGGGSGGGGSERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLTSKLTVDKSRWQQGNVFSCSVMHEALHNHYTQKSLSLSPGK

SEQ ID NO: 176 - protein, 730 aa, synthetic construct
MKHLWFFLLLVAAPRWVLSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVCTLPPSREEMTKNQVSLSCAVKGFYPSDIAVEWESNGQPENNYKTTPPMLDSDGSFFLVSKLTVDKSRWQQGNVFSCSVMHEALHNHYTQKSLSLSPGKGGGGSGGGGSGGGGSDVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSS

SEQ ID NO: 177 - protein, 730 aa, synthetic construct
MKHLWFFLLLVAAPRWVLSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLTSKLTVDKSRWQQGNVFSCSVMHEALHNHYTQKSLSLSPGKGGGGSGGGGSGGGGSDVVMTQSPDSLAVSLGERATINCKSSQSIVHRNGNTYLEWYQQKPGQPPKLLIYKVSNRFSGVPDRFSGSGSGTDFTLTISSLQAEDVAVYYCFQGSHVPYTFGGGTKLEIKGGGGSGGGGSGGGGSQVQLVQSGAEVKKPGASVKVSCKASGYSFTTYTIHWVRQAPGQGLEWIGYINPSSGYAAYNQKFQGRVTLTADKSTSTAYMELSSLRSEDTAVYYCAREKFYGYDYWGQGTTVTVSS

SEQ ID NO: 178 - DNA, 2133 bp, synthetic construct
  source 1..2133: mol_type = other DNA
caagtgcaactagtgcaaagtggtgcagaagtcaagaagcccggcgcttccgtgaaggtatcatgcaaggcctctggatacaccttcaccggctactacatgcattgggtcagacaggcccccggacaaggtctggaatggatgggctggatcaaccctgactccggcggaaccaactacgcccagaagtttcagggacgcgtgaccatgaccagagacacaagcatctccaccgcttatatggagctgaaccggctgagatccgacgacaccgccgtgtactactgtgctagagaccagcctctgggctactgcaccaatggcgtgtgctcctacttcgactactggggccagggaaccctggtgacagtgtcctctgccagcaccaaaggcccttctgtgttccccctggccccttgctctcggagcacctccgagtctaccgctgctctgggctgcctggtgaaggattactttcccgagcctgtgaccgtctcttggaactccggcgccctcacctccggcgtgcacacctttcctgctgtgctgcagtccagcggcctgtactctctgtcctctgtggtgaccgtgccttcttccaacttcggcacacagacctacacctgcaacgtggaccacaagcctagcaataccaaggtagataagacggtggaacggaagtgctgcgtggagtgccctccgtgtcctgcccctcctgtggcaggcccatctgtgtttctgttccctccaaagcccaaggacaccctgatgatctccagaacccctgaggtgacctgtgtggtggtcgatgtgtctcacgaggaccctgaagtgcagttcaactggtacgtggacggagtggaagtgcataacgccaagaccaaacctcgggaagagcagttcaacagcaccttcagagtggtgtccgtgctgaccgtggtacaccaggactggctgaacggcaaagagtacaagtgcaaggtgtctaacaagggcctgcctgctccaatcgagaagacgatttccaaaacaaagggccagcccagagaacctcaggtgtacacactgcctccatctagggaggaaatgaccaagaaccaggtgtccctgacctgtctggtcaagggcttctacccctccgacatcgccgtggaatgggagagcaatggacagcctgagaacaactacaaaaccacacctcctatgctggactctgacggctcattcttcctgtactccaagctgaccgtggataagtccagatggcagcagggcaatgtgttcagctgttctgtgatgcacgaggccctgcacaaccactacacccagaagtccctgtctctcagccctgggaagggcggaggaggctccggaggcggcggctctggaggtggaggcagcgatgtcgtgatgacccaatctcctgatagcctggctgtgtccttgggcgagagagccaccatcaactgcaagtcttctcagtctatcgtgcaccggaacggcaacacctacctcgagtggtaccagcagaaaccaggccaacctcctaagctgctgatctacaaggtgtccaacagattctctggcgtccccgaccggttctccggctccggctccggcaccgacttcaccctgaccatctcctccctgcaggctgaggacgtggccgtgtattattgcttccagggctcccacgtgccctacacctttggcggcggcaccaagctggagatcaagggtggcggcggctctggcggcggcggctccggaggtggtggctcccaagtgcagctggtgcagtccggcgccgaggtgaagaaacctggcgcttctgtgaaagtgtcctgcaaggcctctggctactccttcaccacctatacaatccactgggtgcggcaggcccctggccaaggcctggaatggatcggctacatcaacccctccagtggctacgccgcttacaaccagaagttccagggcagagtgaccctgaccgccgacaagtccacctctacagcctacatggaactgtcctctctgcggtctgaggataccgctgtgtactactgcgctagagagaagttctacggctacgactactggggccagggcaccaccgtgacagtctcctct

SEQ ID NO: 179 - DNA, 642 bp, synthetic construct
  source 1..642: mol_type = other DNA
gatatccaaatgactcaaagtccaagtagtgtgtccgcttccgtgggcgaccgcgtgaccatcacctgtagagcctcgcagggcatctattcttggctggcttggtaccagcagaagcctggcaaggctcctaatctgctgatctacaccgcctctaccctccagtccggcgtgccttcccggttctccggctctggaagcggcaccgacttcaccctgactatctcctctctgcagcccgaggactttgctacctactactgccagcaggccaacatcttccctctgaccttcggcggaggaaccaaagtggaaatcaagcggaccgtggccgcccctagcgtgttcatctttccaccttctgatgaacaactgaagtctggcaccgcttctgtggtgtgcctgctgaacaacttctaccctagagaggccaaagtgcagtggaaggtggacaacgccctgcagtctggcaactcccaagagtccgtcaccgagcaggattctaaggactccacctactccctgtctagcacactgacactgtccaaggccgactacgagaagcacaaggtgtacgcctgcgaagtgacccaccagggcctgagctcccccgtcacaaagtccttcaacagaggcgagtgc

SEQ ID NO: 180 - DNA, 2115 bp, synthetic construct
  source 1..2115: mol_type = other DNA
gatgtggtgatgactcaaagtccagatagtctggccgtgagcctgggcgagagagccaccatcaactgcaagtctagccagagcatcgtgcacagaaacggcaacacctacctggaatggtaccagctgaagcccggtcagcctcctaagctgctgatctacaaggtgtccaacagattctccggagtgcctgatcggttctctggctccggctctggcaccgacttcaccctgaccatctcctctctgcaggctgaggatgtggccgtgtactattgcttccagggctcccatgtgccctacaccttcggcggcggcacaaagctggagatcaagggcggaggcggcagcggcggaggcggcagtggcggtggcggctcccaggtgcagctggtgcagtctggcgccgaggtgaagaagcccggagcttctgtgaaagtgtcctgtaaggcctctggctacagcttcaccacatataccatgcactgggtcagacaggctcctggacaaggactggagtggatgggctacatcaacccttccagcggatacgccgcctacaaccagaagtttcagggcagagtgaccatgaccgctgacaagtccacctctaccgcttacatggaactgagctccctgagatccgaggacactgcagtgtactactgtgccagagagaagttctacggctacgactactggggccagggaacaaccgttaccgtgtcctccggtggcggcggctctggaggcgggggctccggcggcggaggctctgaagtgcagctggtgcagtctggcgcagaagtgaagaaacctggcgagagcctgcggatctcctgcaagggatccggcgattcttttaccacctactggatcggctgggtgcggcagatgcctggcaagggcctggagtggatgggcatcatctaccctggcgactctgataccatctattctccttccttccagggccaagtcaccatctctgccgacaagtccatcagcaccgcctacctgcagtggtcctccctgaaggcctccgacaccgccatgtactactgcacccggggcgacagaggcgtcgactattggggccagggcaccctcgtgaccgtctccagcgcctctacaaaaggcccttccgtcttccctctggctccttcttctaaatctaccagcggcggcaccgctgctctgggctgtctggtgaaggattacttccccgagcccgtgacagtgtcatggaactctggcgccctgacctctggcgtgcatacattccctgctgtgctgcagtcctctggcctgtactccctgagttctgtggtgaccgtgccttcgtcctccctcggcacccagacatacatctgcaacgtgaaccacaagccctccaacaccaaagtcgacaagaaggtggaacctaagtcttgcgacaaaacccacacctgtcctccttgccccgccccggaggccgctggcggacctagcgtgtttctgttccctcctaagcccaaggacacactgatgatctctcggacccctgaggttacctgcgtggtcgtggacgtttcccacgaagatcctgaggtgaagttcaactggtacgtggatggcgtggaagtgcacaacgccaagaccaagcctcgggaggaacagtacaattccacttacagagtggtctctgtgctgaccgtcctgcaccaggactggctgaacggcaaagagtacaagtgcaaggtgtcaaacaaggctctcccagcccctatcgagaagaccatttccaaggctaagggccagcctcgcgagccccaagtgtacaccctgccaccttctagggaagagatgactaagaatcaagtgtcccttacatgcctggtgaaagggttctatccttctgacatcgccgtggagtgggagtctaatggccagccagagaacaactacaagaccacccctcctgtgctggactccgacggctctttcttcctgtactccaagctgacagtggacaagagcagatggcagcagggcaacgtgttttcctgctccgtgatgcacgaagctctgcacaaccactacacccagaagtccctgtccctgtctccgggcaag

SEQ ID NO: 181 - DNA, 642 bp, synthetic construct
  source 1..642: mol_type = other DNA
gatatccaaatgactcaaagtccaagtagtctgtctgcttctgtgggcgacagagtgaccatcacctgccgcgctagccagggcatctctagatggctggcctggtaccagcagaagcccgagaaagcccctaagtccctgatctacgctgcctcttctctgcagtctggcgtgccttctcggttctccggcagcggctccggcaccgattttacactgacaatctccggtctccagcctgaggacttcgccacctactactgccaacagtacaattcctaccctagaacctttggccagggcaccaaggtggaaatcaagcggaccgtggctgctcccagcgtgttcatcttccctccttccgacgaacagctgaagtccggaaccgcctctgtggtgtgcctgctgaacaacttctatcctcgggaagctaaagtgcagtggaaggtcgataacgccctgcagtccggcaactctcaagagtccgtgaccgagcaggactccaaggactctacctactccctgagctccaccctgaccctgagcaaggccgactacgagaagcacaaggtgtacgcctgcgaggtgacccaccagggactgtcctccccagtcacaaagtccttcaacagaggcgagtgt

SEQ ID NO: 182 - DNA, 2190 bp, synthetic construct
  source 1..2190: mol_type = other DNA
atgaagcatctatggttttttctactactagtcgccgctccaagatgggtgctgtctcaggtgcagctggtgcagagcggagccgaagtcaagaagcctggagcttccgtcaaggtgtcctgcaaggcctctggctatacctttaccggctactatatgcactgggtgcggcaggcccctggccaaggcctggaatggatgggctggatcaaccctgattctggcggcaccaactacgcccagaagtttcagggaagagtgaccatgacaagagatacctctatctccaccgcctacatggagctgaacagactgagatctgacgacaccgccgtgtactactgtgctcgggaccagcccctgggatactgcaccaatggcgtgtgctcctacttcgactactggggccaagggaccttggtgacagtgtcctccgcctctaccaagggcccttctgtgttccctctggccccctgttctcggtctacatctgagtccaccgctgctctgggctgcctggtgaaggattactttcctgagcctgtgaccgtgtcttggaactccggcgctctgacctccggcgtgcacaccttccctgccgtgctccaatctagcgggctgtactctctgtcctccgtcgtgaccgttccttcctccaacttcggcacccagacctacacctgcaacgtggatcacaagccctccaacaccaaagtggacaagaccgtcgagcggaagtgctgtgtggaatgtcctccttgcccagctcctcccgtggctggcccatctgtgtttctgttcccacctaagcctaaggacacactgatgatctcccgcacacctgaagtgacatgcgtggtggtggacgtctcccatgaagatcctgaagtgcagttcaactggtacgtggatggcgtggaggtgcacaacgccaagaccaagcctagagaggaacagttcaacagcaccttccgggtggtctccgtgctgacagttgtgcaccaggactggctgaacggcaaagagtacaagtgcaaagtgtctaacaagggcctgcctgcccctatcgagaagaccatcagcaaaaccaagggccagcccagagaacctcaggtgtacaccctgcctccttctagggaagagatgaccaagaatcaggtgtctctgacctgtctggtcaagggcttctacccctccgacatcgccgtggagtgggagtccaacggacagcctgagaacaactacaagacaacccctcctatgctggacagcgacggcagcttcttcctgtacagcaagctgacagtggacaagagccggtggcagcagggcaatgtgttcagctgctccgtgatgcacgaggccctgcacaaccactacacacagaaatctctgagcctcagccccggcaagggcggtggcgggtccggcggcggtggctctggcggcggcggcagcgatgtggtcatgacccagtcccctgactccctggctgtgtccctgggcgagagagctacgatcaactgtaagtcctctcagtccatcgtgcatcggaacggcaacacctacctggaatggtaccagcaaaaacccggccagcctcccaagctgctgatctacaaggtgtccaaccggttctctggcgtgcccgacagattctctggctctggctctggcaccgacttcaccctgaccattagctcactgcaggccgaggacgtagccgtgtactactgcttccagggctctcacgtgccttatacctttggcggcggcaccaaactcgagatcaagggcggtggcggctccggaggaggcggaagcggcggcggaggctcccaagtgcagctggtgcagtccggcgctgaggtgaagaaacctggtgcttccgtgaaggtgtcttgcaaggcctccggctactccttcaccacctacaccatccactgggtcagacaggctccaggccagggactggagtggatcggctacatcaatcctagctctggttatgccgcctacaaccagaagttccagggcagagtgacactgaccgccgataagtccacttctacagcttatatggaactgtcttctttgcggtccgaggacaccgcagtgtactactgcgccagagagaagttctacggctacgactactggggccagggcaccaccgtgaccgtgtcctcc

SEQ ID NO: 183 - DNA, 702 bp, synthetic construct
  source 1..702: mol_type = other DNA
atggtgctacaaactcaagtgtttatcagtttgctgctgtggatctctggcgcctacggcgatatccagatgacccagtccccttcctctgtgtccgcttccgtgggcgatagggtgaccatcacctgtagagcttcccagggcatctactcttggctggcctggtaccagcagaagcctggcaaggctcctaacctgctgatctataccgcctccacactgcaatctggagtgcccagcagattctccggctccggctctggcaccgacttcaccctgaccatctcctctctgcagcctgaggacttcgccacctactactgccagcaggccaacatcttccctctgaccttcggcggcggaacaaaggtggaaattaagcggaccgtggccgctccctctgtctttatctttcctccatccgacgagcagctgaaaagcggcaccgctagcgtggtgtgcctgctgaacaacttctaccctagagaggccaaggtgcagtggaaagtcgacaacgccctgcagtctggcaactcccaagaaagcgtgaccgagcaggactccaaggacagcacctactccctgtcctccaccctcaccctgagcaaggctgattacgagaagcacaaggtttacgcctgcgaggtgacacaccagggactgtcttctcccgtgactaagtcattcaatcggggcgaatgc

SEQ ID NO: 184 - DNA, 2172 bp, synthetic construct
  source 1..2172: mol_type = other DNA
atgaagcatctatggttttttctactactagtggccgcccctagatgggtgctgtccgacgtggtgatgacccagtcccctgactccctggccgtgtccctcggcgagagagccaccatcaactgcaagtcctcccagtctatcgtgcacagaaatggcaacacctacttggagtggtaccagctgaagcctggacagcctcccaagctgctgatctacaaggtctctaaccggttctccggcgtccctgatagattttccggctccggctccggcaccgacttcaccctgaccatctccagcctgcaggctgaggacgtggctgtgtactactgcttccagggctctcacgtgccttacactttcggcggcggcaccaagctggagatcaaggggggcggcggcagcggcggaggcggttctggcggcggcggctctcaggtgcagctggtccagtccggcgccgaggtgaagaagcccggcgcctctgtgaaagtgtcttgcaaggcttccggctacagcttcaccacctacaccatgcactgggtgcggcaggcccctggccagggcctggaatggatgggctacatcaatcccagctctggctacgccgcttacaaccagaagttccagggcagagtgaccatgaccgctgacaagtccacatctaccgcctacatggaactgtcttccctgagaagcgaggacaccgctgtgtactactgcgccagagagaagttctacggctacgactattggggacaaggaaccaccgtgaccgtttccagcggcggcggcggatctggaggcggaggctctggcggcggcggctccgaagtgcagctcgtgcagtccggcgcagaggtgaagaagcctggcgaatctctgcggatctcctgcaagggctccggggatagcttcaccacatactggatcggctgggtcagacagatgcctggaaagggcctggaatggatgggcatcatctaccctggcgactctgacaccatctactctccatctttccaaggccaagtgaccatctctgccgataagtccatctctacagcctacctgcagtggtccagtctgaaagcctccgataccgccatgtactactgcacaagaggagatagaggcgtggactattggggccagggcaccctggtgaccgtgtcttctgcttctaccaaaggcccctccgtgtttcctctggctccttcttccaagtccacctccggcggtacggccgctctgggctgtctggtcaaggactacttccctgagcctgtgaccgtctcttggaactctggagctctgacctctggcgtgcataccttcccagccgtgctgcaatcctccggactgtactcgctgtcttctgtggtgacagtgcccagctcttctctgggaacacagacctacatctgcaacgtgaaccacaagccctcgaacaccaaggtggacaagaaggtggaacctaagtcttgcgacaaaacccacacctgccctccttgtcctgctcccgaggcagctggcggccctagtgtgtttctgttccctcccaagcctaaggacaccttgatgatctctcgcacccctgaggtcacctgtgtggtcgtggacgtgtcccacgaggaccctgaggtgaagtttaactggtacgtggacggcgtggaagtgcacaacgccaagaccaagccccgggaggaacagtacaactccacctaccgggttgtgtccgtgctgacagtgctgcaccaggattggctgaacggaaaagagtacaagtgcaaagtgtctaacaaggccctgcctgctcctatcgagaagacaattagcaaggctaagggtcagcctcgggagcctcaggtgtatacactgccaccttccagggaagagatgacaaagaaccaagtgagcctgacctgtctggtgaagggcttctacccatccgacatcgccgtggaatgggagagtaacggccagcccgagaacaactacaaaaccacacctccagtgctggactccgatggctccttcttcctgtattctaagctgaccgtggacaagtcccggtggcagcagggcaacgtgttctcctgctccgtgatgcatgaagccctgcacaatcactacacccagaaatctctgtccctgtctcctggcaag

SEQ ID NO: 185 - DNA, 702 bp, synthetic construct
  source 1..702: mol_type = other DNA
atggtgctacaaactcaagtgtttatcagtctcctgctgtggatctctggagcctatggcgacatccagatgacccagtctccttccagcctgtctgcctccgtgggcgatagagtgaccatcacctgcagagcctctcagggaatctccaggtggctggcctggtaccagcagaagcctgaaaaagctcctaagtccctgatctacgctgcttcctctctgcaatctggcgtgccttctcggttctccggatctggctctggcaccgacttcaccctgacaatcagcggcctgcagcctgaggattttgctacctactactgccagcagtacaactcctaccccagaacctttggccagggcaccaaagtggaaatcaagcggaccgtggccgctccatccgtgttcatcttccctcccagcgacgagcagctgaagtctggcacagcttctgtcgtgtgcctgctgaacaacttctaccctagagaggccaaggtgcagtggaaggtggataacgccctgcagtccggcaattcccaagagtccgtgactgagcaggactccaaggactccacctactccctgagctctaccctgacactgtccaaggccgactacgagaagcacaaggtgtacgcctgtgaagtgacccaccagggcctgagtagccccgtcaccaagtccttcaaccggggcgagtgc

SEQ ID NO: 186 - protein, 20 aa, Homo sapiens
LLDTRQFLIYLEDTKRCVDA

SEQ ID NO: 187 - protein, 5 aa, synthetic construct
GSGGS

SEQ ID NO: 188 - protein, 4 aa, synthetic construct
GGGS

SEQ ID NO: 189 - protein, 5 aa, synthetic construct
GGGGS

SEQ ID NO: 190 - protein, 4 aa, synthetic construct
LLQG

SEQ ID NO: 191 - protein, 6 aa, synthetic construct
FHENSP

(19) United States
(12) Patent Application Publication
(10) Pub. No.: US 2026/0285997 A1
(43) Pub. Date: Sep. 24, 2026

(54) BISPECIFIC POLYPEPTIDES AND USES THEREOF

(71) Applicant: Currus Biologics Pty Ltd, Melbourne, Victoria (AU)

(72) Inventors: Michael Kershaw, Ocean Grove (AU); Samantha Cobb, Melbourne (AU)

(73) Assignee: Currus Biologics Pty Ltd, Melbourne, Victoria (AU)

(21) Appl. No.: 19/112,495

(22) PCT Filed: Sep. 20, 2023
(86) PCT No.: PCT/AU2023/050902

§ 371 Date: Mar. 17, 2025

(30) Foreign Application Priority Data
Sep. 20, 2022 (AU) 2022902713

(51) Int. Cl. C07K 16/28; A61P 35/00; C07K 16/00

(52) U.S. Cl. CPC C07K 16/2878; A61P 35/00; C07K 16/00; C07K 2317/31; C07K 2317/569; C07K 2317/622; C07K 2317/626

(57) ABSTRACT

The present disclosure relates generally to a bispecific polypeptide, including parts and uses thereof, comprising a first binding domain, which is capable of binding an antigen presenting cell (APC) and a second binding domain, which is capable of binding to a T cell. In a first aspect disclosed herein, there is provided a bispecific polypeptide comprising a first antigen binding protein and a second antigen binding protein, the first antigen binding protein specifically binds to CD40 and wherein the second antigen binding protein specifically binds to a FLAG tag. In a second aspect disclosed herein, there is provided a bispecific polypeptide comprising a first antigen binding protein and a second antigen binding protein, wherein the first antigen binding protein specifically binds to CD206 and wherein the second antigen binding protein specifically binds to a FLAG tag.

### FIELD OF THE INVENTION

The present disclosure relates generally to a bispecific polypeptide, including parts and uses thereof, comprising a first binding domain, which is capable of binding an antigen presenting cell (APC) and a second binding domain, which is capable of binding to a T cell.

### RELATED APPLICATIONS

This application is a national stage filing under 35 U.S.C. 371 of International Patent Application No. PCT/AU2023/050902, filed Sep. 20, 2023, which claims the benefit of Australian application number AU 2022902713, each of which is herein incorporated by reference in its entirety.

### REFERENCE TO AN ELECTRONIC SEQUENCE LISTING

The contents of the electronic sequence listing (C1695700001US00-SEQ-KZM.xml; Size: 194,200 bytes; and Date of Creation: Mar. 12, 2025) is herein incorporated by reference in its entirety.

### BACKGROUND OF THE INVENTION

Adoptive cell transfer (ACT) is demonstrating exciting potential for cancer treatment. In ACT, large numbers of autologous tumor-reactive T cells are generated in vitro before reinfusion to patients. Tumour-reactive T cells can be isolated from blood or tumors and expanded in vitro using stimulation with peptides and/or cytokines.

Methods to improve the reactivity of T cells for ACT include the genetic modification of patient lymphocytes to generate tumor-reactive T cells against most malignancies, including solid cancers and those of the blood. Two main approaches of genetic modification involve genes encoding T cell receptor (TCR) or a chimeric antigen receptor (CAR). A CAR consists of an antibody-derived domain fused with T cell signaling domains that redirects the effector function of T cells against tumor cells. Both approaches can render the T cells tumor-reactive, but the CAR approach, being non-MHC-restricted, is more widely applicable to a broader range of patients.

CARs can take various forms but are typically composed of an extracellular domain consisting of a single-chain variable fragment (scFv) of an antibody specific for a tumour-associated antigen (TAA). This scFv is linked, via hinge and transmembrane domains, to an intracellular region composed of one or more signaling moieties. CARs have been developed with specificity for a range of TAA, including Mesothelin, Her2, CEA, FBP, CD19 and BCMA. The most advanced clinical studies have utilized CARs specific for CD19 for the treatment of B cell leukemia and lymphomas. Since 2017, six CAR T-cell therapies have been approved by the Food and Drug Administration (FDA). All are approved for the treatment of blood cancers, including lymphomas, some forms of leukemia, and, most recently, multiple myeloma.

Attempts to optimize this type of therapy have led to combining CAR T cells with other therapeutic approaches designed to overcome tumour-induced immunosuppression, including co-treatment with α-PD-1 monoclonal antibody, genetic modification of signaling and cytokine pathways, and the use of adjuvants, such as agonistic α-4-1 BB monoclonal antibodies (mAB) and Bispecific T Cell Engagers (BiTEs). While some of these approaches have proved successful in enabling a direct interaction between T cells and cancer cells, which can lead to T cell expansion in hematopoietic cancer cells, significant expansion in solid cancer setting is rarely observed.

Optimization of therapeutic approaches for the delivery of CAR T cells is often exacerbated by difficulties faced in the expansion and manufacture of CAR T cells in vitro due to low lymphocyte counts and poor condition of cells from heavily treated patients. Furthermore, even where sufficient donor cells are available, a significant number of cells are required to be manufactured in order to provide an effective dose of these therapies to patients, while allogeneic cells from healthy donors have been suggested as a solution to this problem, there are a number of issues, such as human leucocyte antigen (HLA) mismatch between the donor and recipient, which can lead to Graft vs. Host Disease (GvHD). There remains, therefore, an urgent need for the development of new reagents to improve the efficacy of ACT, in particular CAR T therapies.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In a first aspect disclosed herein, there is provided a bispecific polypeptide comprising a first antigen binding protein and a second antigen binding protein, wherein the first antigen binding protein specifically binds to CD40 and wherein the second antigen binding protein specifically binds to a FLAG tag.

In an embodiment of the first aspect, the first antigen binding protein binds the outer A-module of the membrane proximal domain (D1a) of CD40, optionally wherein said binding does not prevent, reduce or inhibit CD40-CD40L binding.

In an embodiment of the first aspect, the first antigen binding protein comprises an antigen binding domain comprising:
FR1-CDR1-FR2-CDR2-FR3-CDR3-FR4, and
FR1a-CDR1a-FR2a-CDR2a-FR3a-CDR3a-FR4a,
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the framework regions and/or complementarity determining regions are as described herein, preferably as described in Table 1 below in relation to CD40 binding proteins.

In any embodiment of the first aspect, CDR1, CDR2 and CDR3 refer to complementarity determining regions from the variable heavy chain of an antibody (a VH), CDR1a, CDR2a and CDR3a are complementarity determining regions from the variable light chain of an antibody (a VL), or where CDR1, CDR2 and CDR3 are complementarity determining regions from the VL, CDR1a, CDR2a and CDR3a are complementarity determining regions from VH. In such examples, the CDRs may be referred to as CDRH1, CDRH2, CDRH3, CDRL1, CDRL2 and CDRL3 as the case may be.

Reference herein to a protein or antibody that “binds to” CD40 provides literal support for a protein or antibody that “binds specifically to” or “specifically binds to” CD40.

In any embodiment of the first aspect, the invention provides a first antigen binding protein for binding to CD40, wherein the first antigen binding protein competitively inhibits the binding to CD40 of an antibody or antigen binding fragment thereof, comprising a VH comprising a sequence as set forth in SEQ ID NO: 1 and a VL comprising a sequence as set forth in SEQ ID NO: 2.

In any embodiment of the first aspect, the invention provides a first antigen binding protein with a CDRH1, a CDRH2 and/or a CDRH3 of an antigen binding domain having a variable heavy domain as defined in SEQ ID NO: 1.

In any embodiment of the first aspect, the invention provides a first antigen binding protein with a CDRL1, a CDRL2 and/or a CDRL3 of an antigen binding domain having a variable light chain as defined in of SEQ ID NO: 2.

In any embodiment of the first aspect, the invention provides a first antigen binding protein with a CDR1, a CDR2 and/or a CDR3 of an antigen binding domain having a variable heavy chain as defined in SEQ ID NO: 1 and a variable light chain as defined in SEQ ID NO: 2.

In any embodiment of the first aspect, a first antigen binding protein described herein comprises:
FR1-CDR1-FR2-CDR2-FR3-CDR3-FR4-linker-FR1a-CDR1a-FR2a-CDR2a-FR3a-CDR3a-FR4a,
wherein the sequence of any of the framework regions and/or complementarity determining regions are as described herein, preferably as described in Table 1 below.

As defined herein, the linker may be a chemical, one or more amino acids, or a disulphide bond formed between two cysteine residues.

In any embodiment of the first aspect, a first antigen binding protein comprises:
(i) and (ii):
(i) a VH comprising a complementarity determining region (CDR) 1 comprising or consisting of a sequence as set forth in SEQ ID NO: 3, a CDR2 comprising or consisting of a sequence as set forth in SEQ ID NO: 6 and a CDR3 comprising or consisting of a sequence as set forth in SEQ ID NO: 10, and
(ii) a VL comprising a complementarity determining region (CDR) 1 comprising or consisting of a sequence as set forth in SEQ ID NO: 12, a CDR2 comprising or consisting of a sequence as set forth in SEQ ID NO: 14 and a CDR3 comprising or consisting of a sequence as set forth in SEQ ID NO: 16;
or
(iii) and (iv):
(iii) a VH comprising a complementarity determining region (CDR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 3, a CDR2 comprising or consisting of a sequence at least about 80%, at least 85%, at least 90%, at least 92%, at least 95%, at least 97%, at least 99% identical to a sequence set forth in SEQ ID NO: 6; a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 10, and
(iv) a VL comprising a CDR1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 12, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 14, a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 16;
or
(v) and (vi):
(v) a VH comprising a complementarity determining region (CDR) 1 comprising or consisting of a sequence as set forth in SEQ ID NO: 5, a CDR2 comprising or consisting of a sequence as set forth in SEQ ID NO: 9 and a CDR3 comprising or consisting of a sequence as set forth in SEQ ID NO: 11, and
(vi) a VL comprising a complementarity determining region (CDR) 1 comprising or consisting of a sequence as set forth in SEQ ID NO: 13, a CDR2 comprising or consisting of a sequence as set forth in SEQ ID NO: 15 and a CDR3 comprising or consisting of a sequence as set forth in SEQ ID NO: 16;
or
(vii) and (viii):
(vii) a VH comprising a complementarity determining region (CDR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 5, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 9; a CDR3 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 11, and
(viii) a VL comprising a CDR1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 13, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 15, a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 16;
or
(ix) and (x):
(ix) a VH comprising a complementarity determining region (CDR) 1 comprising or consisting of a sequence as set forth in SEQ ID NO: 4, a CDR2 comprising or consisting of a sequence as set forth in SEQ ID NO: 7 and a CDR3 comprising or consisting of a sequence as set forth in SEQ ID NO: 10, and
(x) a VL comprising a complementarity determining region (CDR) 1 comprising or consisting of a sequence as set forth in SEQ ID NO: 12, a CDR2 comprising or consisting of a sequence as set forth in SEQ ID NO: 14 and a CDR3 comprising or consisting of a sequence as set forth in SEQ ID NO: 16;
or
(xi) and (xii):
(xi) a VH comprising a complementarity determining region (CDR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 4, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 7; a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 10, and
(xii) a VL comprising a CDR1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 12, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 14, a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 16;
or
(xiii) and (xiv):
(xiii) a VH comprising a complementarity determining region (CDR) 1 comprising or consisting of a sequence as set forth in SEQ ID NO: 4, a CDR2 comprising or consisting of a sequence as set forth in SEQ ID NO: 8 and a CDR3 comprising or consisting of a sequence as set forth in SEQ ID NO: 10, and
(xiv) a VL comprising a complementarity determining region (CDR) 1 comprising or consisting of a sequence as set forth in SEQ ID NO: 12, a CDR2 comprising or consisting of a sequence as set forth in SEQ ID NO: 14 and a CDR3 comprising or consisting of a sequence as set forth in SEQ ID NO: 16;
or
(xv) and (xvi):
(xv) a VH comprising a complementarity determining region (CDR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 4, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 8; a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 10, and
(xvi) a VL comprising a CDR1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 12, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 14, a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 16.

In any embodiment of the first aspect, the first antigen binding protein comprises a heavy chain variable domain comprising or consisting of the amino acid sequence as set forth in SEQ ID NO: 1, or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto. Optionally, the heavy chain variable domain of the first antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEQ ID NO: 1; optionally wherein the amino acid substitutions are not in the CDRs and/or the antigen binding protein retains the ability to bind to CD40.

In any embodiment of the first aspect, the first antigen binding protein comprises a light chain variable domain comprising or consisting of the amino acid sequence as set forth in SEQ ID NO: 2; or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto. Optionally, the light chain variable domain of the first antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEQ ID NO: 2; optionally wherein the amino acid substitutions are not in the CDRs and/or the antigen binding protein retains the ability to bind to CD40.

In any embodiment of the first aspect, the first antigen binding protein comprises a heavy chain variable domain comprising or consisting of the amino acid sequence as set forth in SEQ ID NO: 1, or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto, and a light chain variable domain comprising or consisting of the amino acid sequence as set forth in SEQ ID NO: 2, or sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto. Optionally, the heavy and/or light chain variable domain of the first antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEQ ID NO: 1 or 2, respectively; optionally wherein the amino acid substitutions are not in the CDRs and/or the antigen binding protein retains the ability to bind to CD40.

In any embodiment of the first aspect, the first antigen binding protein comprises, consists essentially of or consists of the amino acid sequence of (in order of N to C terminus or C to N terminus) SEQ ID NO: 1 and 2.

In any embodiment of the first aspect, the first antigen binding protein described herein may have from N to C terminus a VH then VL, or a VL then VH, or any CDR 1, 2 and 3 defined herein as VH then any CDR 1, 2 and 3 defined herein as VL, or any CDR 1, 2 and 3 defined herein as VL then any CDR 1, 2 and 3 defined herein as VH.

In any embodiment of the first aspect, the first antigen binding protein may be in the form of:
(i) a single domain antibody (sdAb);
(ii) a single chain Fv fragment (scFv);
(iii) a dimeric scFv (di-scFv); or
(iv) one of (ii) or (iii) linked to a constant region of an antibody, Fc or a heavy chain constant domain (CH) 2 and/or CH3.

Further, as described herein, in any embodiment of the first aspect, the first antigen binding protein may be in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody;
(viii) one of (i) to (vii) linked to a constant region of an antibody, Fc or a heavy chain constant domain (CH) 2 and/or CH3.

In any embodiment of the first aspect, the first antigen binding protein may be in the form of an immunoglobulin G molecule (IgG). Optionally, in accordance with any embodiment of the first aspect of the invention, the first antigen binding protein may be in the form of an IgG or heterodimeric Fab-Fc, and the second antigen binding protein may be in the form of an antigen binding fragment of an IgG, such as an scFv.

In a second aspect disclosed herein, there is provided a bispecific polypeptide comprising a first antigen binding protein and a second antigen binding protein, wherein the first antigen binding protein specifically binds to CD206 and wherein the second antigen binding protein specifically binds to a FLAG tag.

In any embodiment of the second aspect, the first antigen binding protein binds to CD206 expressed on the surface of an immune cell, preferably an antigen presenting cell, more preferably a professional antigen presenting cell. Preferably, the first antigen binding protein binds to CD206 expressed on the surface of a professional antigen presenting cell selected from: a dendritic cell, macrophage, B-cell, epithelial cell, most preferably a dendritic cell and even more preferably wherein the professional antigen presenting cell is not a tumor cell.

In an embodiment of the first aspect, the first antigen binding protein binds to human macrophage B111 antigen, optionally wherein the B111 antigen comprises the amino acid sequence as set forth om SEQ ID NO: 186.

In any embodiment of the second aspect, the first antigen binding protein comprises an antigen binding domain comprising:
FR1-CDR1-FR2-CDR2-FR3-CDR3-FR4, and
FR1a-CDR1a-FR2a-CDR2a-FR3a-CDR3a-FR4a,
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the framework regions and/or complementarity determining regions are as described herein, preferably as described in Table 1 below in relation to CD206 binding proteins.

In this embodiment of the second aspect, CDR1, CDR2 and CDR3 refer to complementarity determining regions from the variable heavy chain of an antibody (a VH), CDR1a, CDR2a and CDR3a are complementarity determining regions from the variable light chain of an antibody (a VL), or where CDR1, CDR2 and CDR3 are complementarity determining regions from the VL, CDR1a, CDR2a and CDR3a are complementarity determining regions from VH. In such examples, the CDRs may be referred to as CDRH1, CDRH2, CDRH3, CDRL1, CDRL2 and CDRL3 as the case may be.

Reference herein to a protein or antibody that “binds to” CD206 provides literal support for a protein or antibody that “binds specifically to” or “specifically binds to” CD206.

In any embodiment of the second aspect, the invention provides a first antigen binding protein for binding to CD206, wherein the first antigen binding protein competitively inhibits the binding to CD206 of an antibody comprising a VH comprising a sequence as set forth in SEQ ID NO: 33 and a VL comprising a sequence as set forth in SEQ ID NO: 34.

In any embodiment of the second aspect, the invention provide a first antigen binding protein with a CDRH1, a CDRH2 and/or a CDRH3 of an antigen binding domain having a variable heavy domain as defined in SEQ ID NO: 33.

In any embodiment of the second aspect, the invention provides a first antigen binding protein with a CDRL1, a CDRL2 and/or a CDRL3 of an antigen binding domain having a variable light chain as defined in of SEQ ID NO: 34.

In any embodiment of the second aspect, the invention provides a first antigen binding protein with a CDR1, a CDR2 and/or a CDR3 of an antigen binding domain having a variable heavy chain as defined in SEQ ID NO: 33 and a variable light chain as defined in SEQ ID NO: 34.

In any embodiment of the second aspect, a first antigen binding protein described herein comprises:
FR1-CDR1-FR2-CDR2-FR3-CDR3-FR4-linker-FR1a-CDR1a-FR2a-CDR2a-FR3a-CDR3a-FR4a,
wherein the sequence of any of the framework regions and/or complementarity determining regions are as described herein, preferably as described in Table 1 below.

As defined herein, the linker may be a chemical, one or more amino acids, or a disulphide bond formed between two cysteine residues.

In any embodiment of the second aspect, a first antigen binding protein comprises an antigen binding domain comprising:
(i) and (ii):
(i) a VH comprising a complementarity determining region (CDR) 1 comprising or consisting of a sequence as set forth in SEQ ID NO: 35, a CDR2 comprising or consisting of a sequence as set forth in SEQ ID NO: 38 and a CDR3 comprising or consisting of a sequence as set forth in SEQ ID NO: 42, and
(ii) a VL comprising a complementarity determining region (CDR) 1 comprising or consisting of a sequence as set forth in SEQ ID NO: 44, a CDR2 comprising or consisting of a sequence as set forth in SEQ ID NO: 46 and a CDR3 comprising or consisting of a sequence as set forth in SEQ ID NO: 48;
or
(iii) and (iv):
(iii) a VH comprising a complementarity determining region (CDR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 35, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 38; a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 42, and
(iv) a VL comprising a CDR1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 44, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 46, a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 48;
or
(v) and (vi):
(v) a VH comprising a complementarity determining region (CDR) 1 comprising or consisting of a sequence as set forth in SEQ ID NO: 37, a CDR2 comprising or consisting of a sequence as set forth in SEQ ID NO: 41 and a CDR3 comprising or consisting of a sequence as set forth in SEQ ID NO: 43, and
(vi) a VL comprising a complementarity determining region (CDR) 1 comprising or consisting of a sequence as set forth in SEQ ID NO: 45, a CDR2 comprising or consisting of a sequence as set forth in SEQ ID NO: 47 and a CDR3 comprising or consisting of a sequence as set forth in SEQ ID NO: 48;
or
(vii) and (viii):
(vii) a VH comprising a complementarity determining region (CDR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 37, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 41; a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 43, and
(viii) a VL comprising a CDR1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 45, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 47, a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 48;
or
(ix) and (x):
(ix) a VH comprising a complementarity determining region (CDR) 1 comprising or consisting of a sequence as set forth in SEQ ID NO: 36, a CDR2 comprising or consisting of a sequence as set forth in SEQ ID NO: 39 and a CDR3 comprising or consisting of a sequence as set forth in SEQ ID NO: 42, and
(x) a VL comprising a complementarity determining region (CDR) 1 comprising or consisting of a sequence as set forth in SEQ ID NO: 44, a CDR2 comprising or consisting of a sequence as set forth in SEQ ID NO: 46 and a CDR3 comprising or consisting of a sequence as set forth in SEQ ID NO: 48;
or
(xi) and (xii):
(xi) a VH comprising a complementarity determining region (CDR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 36, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 39; a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 42, and
(xii) a VL comprising a CDR1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 44, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 46, a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 48;
or
(xiii) and (xiv):
(xiii) a VH comprising a complementarity determining region (CDR) 1 comprising or consisting of a sequence as set forth in SEQ ID NO: 36, a CDR2 comprising or consisting of a sequence as set forth in SEQ ID NO: 40 and a CDR3 comprising or consisting of a sequence as set forth in SEQ ID NO: 42, and
(xiv) a VL comprising a complementarity determining region (CDR) 1 comprising or consisting of a sequence as set forth in SEQ ID NO: 44, a CDR2 comprising or consisting of a sequence as set forth in SEQ ID NO: 46 and a CDR3 comprising or consisting of a sequence as set forth in SEQ ID NO: 48;
or
(xv) and (xvi):
(xv) a VH comprising a complementarity determining region (CDR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 36, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 40; a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 42, and
(xvi) a VL comprising a CDR1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 44, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 46, a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 48.

In any embodiment of the second aspect, the first antigen binding protein comprises a heavy chain variable domain comprising or consisting of the amino acid sequence as set forth in SEQ ID NO: 33, or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto. Optionally, the heavy chain variable domain of the first antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEQ ID NO: 33; optionally wherein the amino acid substitutions are not in the CDRs and/or the antigen binding protein retains the ability to bind to CD206.

In any embodiment of the second aspect, the first antigen binding protein comprises a light chain variable domain comprising or consisting of the amino acid sequence as set forth in SEQ ID NO: 34; or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto. Optionally, the light chain variable domain of the first antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEQ ID NO: 34; optionally wherein the amino acid substitutions are not in the CDRs and/or the antigen binding protein retains the ability to bind to CD206.

In any embodiment of the second aspect, the first antigen binding protein comprises a heavy chain variable domain comprising or consisting of the amino acid sequence as set forth in SEQ ID NO: 33, or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto, and a light chain variable domain comprising the amino acid sequence as set forth in SEQ ID NO: 34, or sequences at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto. Optionally, the heavy and light chain variable domains of the first antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEQ ID NO: 33 or 34, respectively; optionally wherein the amino acid substitutions are not in the CDRs and/or the antigen binding protein retains the ability to bind to CD206.

In any embodiment of the second aspect, the first antigen binding protein comprises, consists essentially of or consists of an amino acid sequence of (in order of N to C terminus or C to N terminus) SEQ ID NO: 33 and 34.

In any embodiment of the second aspect, the first antigen binding protein described herein may have from N to C terminus a VH then VL, or a VL then VH, or any CDR 1, 2 and 3 defined herein as VH then any CDR 1, 2 and 3 defined herein as VL, or any CDR 1, 2 and 3 defined herein as VL then any CDR 1, 2 and 3 defined herein as VH.

In any embodiment of the second aspect, the first antigen binding protein may be in the form of:
(i) a single domain antibody (sdAb);
(ii) a single chain Fv fragment (scFv);
(iii) a dimeric scFv (di-scFv); or
(iv) one of (ii) or (iii) linked to a constant region of an antibody, Fc or a heavy chain constant domain (CH) 2 and/or CH3.

Further, as described herein, the first antigen binding protein may be in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody;
(viii) one of (i) to (vii) linked to a constant region of an antibody, Fc or a heavy chain constant domain (CH) 2 and/or CH3.

In any embodiment of the second aspect, the first antigen binding protein may be in IgG format. In any embodiment of the second aspect, the first antigen binding protein may be in the form of an immunoglobulin G molecule (IgG). Optionally, in accordance with any embodiment of the second aspect of the invention, the first antigen binding protein may be in the form of an IgG or heterodimeric Fab-Fc, and the second antigen binding protein may be in the form of an antigen binding fragment of an IgG, such as an scFv.

In any aspect, including the first and second aspects, the second antigen binding protein is for binding to a FLAG tag, the second antigen binding protein comprising an antigen binding domain comprising:
FR1-CDR1-FR2-CDR2-FR3-CDR3-FR4, and
FR1a-CDR1a-FR2a-CDR2a-FR3a-CDR3a-FR4a,
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the framework regions and/or complementarity determining regions are as described herein, preferably as described in Table 1 below.

In any embodiment, CDR1, CDR2 and CDR3 refer to complementarity determining regions from the variable heavy chain of an antibody (a VH), CDR1a, CDR2a and CDR3a are complementarity determining regions from the variable light chain of an antibody (a VL), or where CDR1, CDR2 and CDR3 are complementarity determining regions from the VL, CDR1a, CDR2a and CDR3a are complementarity determining regions from VH. In such examples, the CDRs may be referred to as CDRH1, CDRH2, CDRH3, CDRL1, CDRL2 and CDRL3 as the case may be.

In any embodiment of any aspect of the invention the second antigen binding protein is capable of specifically binding to a FLAG tag or variant thereof (such as defined in SEQ ID NOs: 80 and 99 or as otherwise defined herein). The antigen binding proteins of the invention are preferably capable of specifically binding to protein domains comprising multiples of the FLAG tag sequences (eg 2×FLAG, 3×FLAG etc).

In any embodiment of any aspect of the invention, the second antigen binding protein is for specifically binding to a peptide tag that contains or consists of the sequence DYK, preferably the sequence DYKD (SEQ ID NO: 80). In addition to these sequences, other amino acids can be present in the FLAG tag bound by the second antigen binding protein, preferably wherein the additional amino acids in the FLAG tag are hydrophilic amino acids for example R (Arg), D (Asp), E (Glu) and K (Lys) and/or amino acids with aromatic side chains for example Y (Tyr), F (Phe), H (His) and W (Trp).

In preferred embodiments of any aspect of the invention the second antigen binding protein is capable of specifically binding to a FLAG-tag that contains, comprises or consists of the sequence GDYKDDDDKG (SEQ ID NO: 98), DYKDDDDK (SEQ ID NO: 99), MDYKDDDDK (SEQ ID NO: 100), DFKDDDK (SEQ ID NO: 101), DYKAFDNL (SEQ ID NO: 102), DYKDHDG (SEQ ID NO: 103), MDFKDDDDK (SEQ ID NO: 104), MDYKAFDNL (SEQ ID NO: 105), DYKDHDI (SEQ ID NO: 106), DYKDH (SEQ ID NO: 107), DYKDD (SEQ ID NO: 108), DYKDHD (SEQ ID NO: 109) and/or DYKDDD (SEQ ID NO: 110). The most preferred sequence is DYKDDDDK (SEQ ID NO: 99).

As used herein, the term FLAG-tag also encompasses modified FLAG-tags, such as those derived from the FLAG-tags described above, especially the tag with the sequence DYKDDDDK, by amino acid insertion, deletion or substitution.

Reference herein to a protein or antibody that “binds to” FLAG tag provides literal support for a protein or antibody that “binds specifically to” or “specifically binds to” a FLAG tag.

In any embodiment, the FLAG tag is present at the N-terminus, the C-terminus or within the protein to which the bispecific polypeptide of the invention is capable of binding to, binds to or specifically binds to.

In any embodiment of any aspect of the invention, the second antigen binding protein competitively inhibits the binding to a FLAG tag of an antibody:
comprising a VH comprising a sequence as set forth in SEQ ID NO: 70 and a VL comprising a sequence as set forth in SEQ ID NO: 75;
comprising a VH comprising a sequence as set forth in SEQ ID NO: 71 and a VL comprising a sequence as set forth in SEQ ID NO: 76;
comprising a VH comprising a sequence as set forth in SEQ ID NO: 71 and a VL comprising a sequence as set forth in SEQ ID NO: 77;
comprising a VH comprising a sequence as set forth in SEQ ID NO: 71 and a VL comprising a sequence as set forth in SEQ ID NO: 78;
comprising a VH comprising a sequence as set forth in SEQ ID NO: 71 and a VL comprising a sequence as set forth in SEQ ID NO: 79;
comprising a VH comprising a sequence as set forth in SEQ ID NO: 72 and a VL comprising a sequence as set forth in SEQ ID NO: 76;
comprising a VH comprising a sequence as set forth in SEQ ID NO: 72 and a VL comprising a sequence as set forth in SEQ ID NO: 77;
comprising a VH comprising a sequence as set forth in SEQ ID NO: 72 and a VL comprising a sequence as set forth in SEQ ID NO: 78;
comprising a VH comprising a sequence as set forth in SEQ ID NO: 72 and a VL comprising a sequence as set forth in SEQ ID NO: 79;
comprising a VH comprising a sequence as set forth in SEQ ID NO: 73 and a VL comprising a sequence as set forth in SEQ ID NO: 76;
comprising a VH comprising a sequence as set forth in SEQ ID NO: 73 and a VL comprising a sequence as set forth in SEQ ID NO: 77;
comprising a VH comprising a sequence as set forth in SEQ ID NO: 73 and a VL comprising a sequence as set forth in SEQ ID NO: 78;
comprising a VH comprising a sequence as set forth in SEQ ID NO: 73 and a VL comprising a sequence as set forth in SEQ ID NO: 79;
comprising a VH comprising a sequence as set forth in SEQ ID NO: 74 and a VL comprising a sequence as set forth in SEQ ID NO: 76;
comprising a VH comprising a sequence as set forth in SEQ ID NO: 74 and a VL comprising a sequence as set forth in SEQ ID NO: 77;
comprising a VH comprising a sequence as set forth in SEQ ID NO: 74 and a VL comprising a sequence as set forth in SEQ ID NO: 78; or
comprising a VH comprising a sequence as set forth in SEQ ID NO: 74 and a VL comprising a sequence as set forth in SEQ ID NO: 79.

In any embodiment, the second antigen binding protein comprises a CDRH1, a CDRH2 and/or a CDRH3 of an antigen binding domain having a variable heavy chain as defined in any one of SEQ ID NOs: 71 to 74.

In any embodiment, the second antigen binding protein comprises a CDRL1, a CDRL2 and/or a CDRL3 of an antigen binding domain having a variable light chain as defined in any one of SEQ ID NOs: 76 to 79.

In any embodiment, the second antigen binding protein comprises a CDR1, a CDR2 and/or a CDR3 of an antigen binding domain having a variable heavy chain as defined in any one of SEQ ID NOs: 71 to 74 and a variable light chain as defined in any one of SEQ ID NOs: 76 to 79.

In any embodiment of any aspect, a second antigen binding protein described herein comprises:
FR1-CDR1-FR2-CDR2-FR3-CDR3-FR4-linker-FR1a-CDR1a-FR2a-CDR2a-FR3a-CDR3a-FR4a,
wherein the sequence of any of the framework regions and/or complementarity determining regions are as described herein, preferably as described in Table 1 below.

As defined herein, the linker may be a chemical, one or more amino acids, or a disulphide bond formed between two cysteine residues.

In any embodiment of any aspect of the invention, the second antigen binding domain comprises an antigen binding domain comprising:
(a) and (b):
(a) a VH comprising a CDR1 comprising or consisting of an amino acid sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence as set forth in SEQ ID NO: 81; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 82; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 83, and
(b) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NOs: 90; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 91; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 92;
or
(c) and (d):
(c) a VH comprising a complementarity determining region CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NOs: 111 or 112; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 113; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 114, and
(d) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 120; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 121; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 92;
or
(e) and (f):
(e) a VH comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NOs: 111 or 112; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 113; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 114, and
(f) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 133; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 121; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 92;
(g) and (h):
(g) a VH comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NOs: 111 or 156; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 157; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 114, and
(h) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 120; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 121; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 92;
or
(i) and (j):
(i) a VH comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NOs: 111 or 156; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 157; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 114, and
(j) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NOs: 133; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 121; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 92;
or
(k) and (l):
(k) a VH comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 126; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 127; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 114, and
(l) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 120; a CDR2 comprising or consisting of an amino acid sequence of at least about 80%, at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 121; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 92; or
(m) and (n):
(m) a VH comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 126; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 127; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 114, and
(n) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 133; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 121; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 92;
or
(o) and (p):
(o) a VH comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 111; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 132; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 114, and
(p) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 133; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 121; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 92;
or
(q) and (r):
(q) a VH comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 126; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 127; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 114, and
(r) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 133; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 121; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 92.

In an embodiment of any aspect of the invention, the second antigen binding protein comprises an antigen binding domain comprising:
(a) and (b):
(a) a VH comprising a CDR1 comprising or consisting of an amino acid sequence of SEQ ID NO: 81; a CDR2 comprising or consisting of an amino acid sequence of SEQ ID NO: 82; a CDR3 comprising or consisting of an amino acid sequence of SEQ ID NO: 83, and
(b) a VL comprising a CDR1 comprising or consisting of an amino acid sequence SEQ ID NO: 90; a CDR2 comprising or consisting of an amino acid sequence of SEQ ID NO: 91; a CDR3 comprising or consisting of an amino acid sequence SEQ ID NO: 92;
or
(c) and (d):
(c) a VH comprising a CDR1 comprising or consisting of an amino acid sequence of SEQ ID NOs: 111 or 112; a CDR2 comprising or consisting of an amino acid sequence of SEQ ID NO: 113; a CDR3 comprising or consisting of an amino acid sequence of SEQ ID NO: 114, and
(d) a VL comprising a CDR1 comprising or consisting of an amino acid sequence of SEQ ID NO: 120; a CDR2 comprising or consisting of an amino acid sequence of SEQ ID NO: 121; a CDR3 comprising or consisting of an amino acid sequence of SEQ ID NO: 92;
or
(e) and (f):
(e) a VH comprising a CDR1 comprising or consisting of an amino acid sequence of SEQ ID NOs: 111 or 112; a CDR2 comprising or consisting of an amino acid sequence of SEQ ID NO: 113; a CDR3 comprising or consisting of an amino acid sequence of SEQ ID NO: 114, and
(f) a VL comprising a CDR1 comprising or consisting of an amino acid sequence of SEQ ID NO: 133; a CDR2 comprising or consisting of an amino acid sequence SEQ ID NO: 121; a CDR3 comprising or consisting of an amino acid sequence of SEQ ID NO: 92;
or
(g) and (h):
(g) a VH comprising a CDR1 comprising or consisting of an amino acid sequence of SEQ ID NOs: 111 or 156; a CDR2 comprising or consisting of an amino acid sequence of SEQ ID NO: 157; a CDR3 comprising or consisting of an amino acid sequence of SEQ ID NO: 114, and
(h) a VL comprising a CDR1 comprising or consisting of an amino acid sequence of SEQ ID NO: 120; a CDR2 comprising or consisting of an amino acid sequence of SEQ ID NO: 121; a CDR3 comprising or consisting of an amino acid sequence of SEQ ID NO: 92;
or
(i) and (j):
(i) a VH comprising a CDR1 comprising or consisting of an amino acid sequence of SEQ ID NOs: 111 or 156; a CDR2 comprising or consisting of an amino acid sequence of SEQ ID NO: 157; a CDR3 comprising or consisting of an amino acid sequence of SEQ ID NO: 114, and
(j) a VL comprising a CDR1 comprising or consisting of an amino acid sequence of SEQ ID NO: 133; a CDR2 comprising or consisting of an amino acid sequence of SEQ ID NO: 121; a CDR3 comprising or consisting of an amino acid sequence of SEQ ID NO: 92;
or
(k) and (l):
(k) a VH comprising a CDR1 comprising or consisting of an amino acid sequence of SEQ ID NO: 126; a CDR2 comprising or consisting of an amino acid sequence of SEQ ID NO: 127; a CDR3 comprising or consisting of an amino acid sequence of SEQ ID NO: 114, and
(l) a VL comprising a CDR1 comprising or consisting of an amino acid sequence of SEQ ID NO: 120; a CDR2 comprising or consisting of an amino acid sequence of SEQ ID NO: 121; a CDR3 comprising or consisting of an amino acid sequence of SEQ ID NO: 92;
or
(m) and (n):
(m) a VH comprising a CDR1 comprising or consisting of an amino acid sequence of SEQ ID NO: 126; a CDR2 comprising or consisting of an amino acid sequence of SEQ ID NO: 127; a CDR3 comprising or consisting of an amino acid sequence of SEQ ID NO: 114, and
(n) a VL comprising a CDR1 comprising or consisting of an amino acid sequence of SEQ ID NO: 133; a CDR2 comprising or consisting of an amino acid sequence of SEQ ID NO: 121; a CDR3 comprising or consisting of an amino acid sequence of SEQ ID NO: 92;
or
(o) and (p):
(o) a VH comprising a CDR1 comprising or consisting of an amino acid sequence of SEQ ID NO: 111; a CDR2 comprising or consisting of an amino acid sequence of SEQ ID NO: 132; a CDR3 comprising or consisting of an amino acid sequence of SEQ ID NO: 114, and
(p) a VL comprising a CDR1 comprising or consisting of an amino acid sequence of SEQ ID NO: 133; a CDR2 comprising or consisting of an amino acid sequence of SEQ ID NO: 121; a CDR3 comprising or consisting of an amino acid sequence of SEQ ID NO: 92;
or
(q) and (r):
(q) a VH comprising a CDR1 comprising or consisting of an amino acid sequence of SEQ ID NO: 126; a CDR2 comprising or consisting of an amino acid sequence of SEQ ID NO: 127; a CDR3 comprising or consisting of an amino acid sequence of SEQ ID NO: 114, and
(r) a VL comprising a CDR1 comprising or consisting of an amino acid sequence of SEQ ID NO: 133; a CDR2 comprising or consisting of an amino acid sequence of SEQ ID NO: 121; a CDR3 comprising or consisting of an amino acid sequence of SEQ ID NO: 92.

In any embodiment of any aspect of the invention, FR1, FR2, FR3 and FR4 may refer to framework regions from the variable heavy chain of an antibody (a VH), FR1a, FR2a, FR3a and FR4a may refer to framework regions from the variable light chain of an antibody (a VL), or where FR1, FR2, FR3 and FR4 are framework regions from the VL, FR1a, FR2a, FR3a and FR4a are framework regions from VH. In such examples, the FRs may be referred to as FR H1, FR H2, FR H3, FR H4, FR L1, FR L2, FR L3 and FR L4 as the case may be.

In any embodiment of any aspect of the invention, the second antigen binding protein comprises an antigen binding domain with a FR H1, a FR H2, a FR H3 and/or a FR H4 from a human germline, wherein the human germline is IGHV1-46*01 or IGHV7-4-1*02.

In any embodiment of any aspect of the invention, the second antigen binding protein comprises an antigen binding domain with a FR L1, a FR L2, a FR L3 and/or a FR L4 from a human germline, wherein the human germline is IGKV2-30*01 or IGKV4-1*01.

In any embodiment of any aspect of the invention, the second antigen binding protein comprises an antigen binding domain with a FR H1, a FR H2, a FR H3 and/or a FR H4 from a human germline, wherein the human germline is IGHV1-46*01 or IGHV7-4-1*02, and a FR L1, a FR L2, a FR L3 and/or a FR L4 from a human germline, wherein the human germline is IGKV2-30*01 or IGKV4-1*01.

In any embodiment of any aspect of the invention, the second antigen binding protein comprises an antigen binding domain comprising a VH that has a greater than 80%, 81%, 82%, 83%, 84%, 85%, 86%, 87%, 88%, 89%, 90%, 91%, 92% identity to human, preferably the % identity to human is calculated as described in Example 2, and/or a VL that has a greater than 80%, 81%, 82%, 8%, 84%, 85%, 86%, 87%, 88%, 89%, 90%, 91%, 92% identity to human, preferably the % identity to human is calculated as described in Example 2.

In an embodiment of any aspect of the invention, the second antigen binding protein comprises an antigen binding domain with a FR H1, a FR H2, a FR H3 and/or a FR H4 of an antigen binding domain having a variable heavy chain as defined in any one of SEQ ID NOs: 71 to 74.

In any embodiment of any aspect of the invention, the second antigen binding protein comprises an antigen binding domain with a FR L1, a FR L2, a FR L3 and/or a FR L4 of an antigen binding domain having a variable light chain as defined in any one of SEQ ID NOs: 76 to 79.

In any embodiment of any aspect of the invention, the second antigen binding protein comprises an antigen binding domain comprising a FR1, a FR2, a FR3 and/or a FR4 of an antigen binding domain having a variable heavy chain as defined in any one of SEQ ID NOs: 71 to 74, and a FR1, a FR2, a FR3 and/or a FR4 of an antigen binding domain having a variable light chain as defined in any one of SEQ ID NOs: 76 to 79.

In any embodiment of any aspect of the invention, the second antigen binding protein comprises an antigen binding domain, wherein the antigen binding domain comprises a VH comprising a complementarity determining region (CDR) 1, CDR2 and CDR3 as defined in any (a) above and a VL comprising a CDR1, CDR2 and CDR3 as defined in any (b) above, and:
A. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 134, 135, 136 and 137 respectively, and for VL FRs the reference sequences are set forth in SEQ ID NO: 138, 139, 140 and 97 respectively;
B. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 84, 85, 87 and 89 respectively, and for VL FRs the reference sequences are set forth in SEQ ID NO: 138, 139, 140 and 97 respectively;
or
C. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 84, 85, 87 and 89 respectively, and for VL FRs the reference sequences are set forth in SEQ ID NO: 153, 154, 155 and 97 respectively;
or
D. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 84, 85, 87 and 89 respectively, and for VL FRs the reference sequences are set forth in SEQ ID NO: 93, 95, 96 and 97 respectively;
or
E. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 85, 87 and 89 respectively, and for VL FRs the reference sequences are set forth in SEQ ID NO: 93, 94, 96 and 97 respectively;
or
F. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 86, 88 and 89 respectively, and for VL FRs the reference sequences are set forth in SEQ ID NO: 138, 139, 140 and 97 respectively;
or
G. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 84, 86, 88 and 89 respectively, and for VL FRs the reference sequences are set forth in SEQ ID NO: 153, 154, 155 and 97 respectively;
or
H. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 86, 88 and 89 respectively, and for VL FRs the reference sequences are set forth in SEQ ID NO: 93, 95, 96 and 97 respectively;
or
I. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 84, 86, 88 and 89 respectively, and for VL FRs the reference sequences are set forth in SEQ ID NO: 93, 94, 96 and 97 respectively;
or
J. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 149, 85, 151 and 89 respectively, and for VL FRs the reference sequences are set forth in SEQ ID NO: 138, 139, 140 and 97 respectively;
or
K. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 149, 85, 151 and 89 respectively, and for VL FRs the reference sequences are set forth in SEQ ID NO: 153, 154, 155 and 97 respectively;
or
L. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 149, 85, 151 and 89 respectively, and for VL FRs the reference sequences are set forth in SEQ ID NO: 93, 95, 96 and 97 respectively;
or
M. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 149, 85, 151 and 89 respectively, and for VL FRs the reference sequences are set forth in SEQ ID NO: 93, 94, 96 and 97 respectively;
or
N. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 149, 150, 152 and 89 respectively, and for VL FRs the reference sequences are set forth in SEQ ID NO: 138, 139, 140 and 97 respectively;
or
O. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 149, 150, 152 and 89 respectively, and for VL FRs the reference sequences are set forth in SEQ ID NO: 153, 154, 155 and 97 respectively;
or
P. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 149, 150, 152 and 89 respectively, and for VL FRs the reference sequences are set forth in SEQ ID NO: 93, 95, 96 and 97 respectively;
or
Q. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 149, 150, 152 and 89 respectively, and for VL FRs the reference sequences are set forth in SEQ ID NO: 93, 94, 96 and 97 respectively.

In any embodiment, the second antigen binding protein comprises an antigen binding domain, wherein the antigen binding domain comprises VH comprising a complementarity determining region (CDR) 1, CDR2 and CDR3 as defined in any of (c) above and a VL comprising a CDR1, CDR2 and CDR3 as defined in any of (d) above, and:
A. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NO: 115, 116, 118 and 89 respectively, and for VL FRs the reference sequences are set forth in SEQ ID NO: 122, 124, 125 and 127 respectively;
or
B. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NO: 115, 116, 118 and 89 respectively, and for VL FRs the reference sequences are set forth in SEQ ID NO: 122, 123, 125 and 127 respectively;
or
C. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NO: 115, 117, 119 and 89 respectively, and for VL FRs the reference sequences are set forth in SEQ ID NO: 122, 124, 125 and 127 respectively;
or
D. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NO: 115, 117, 119 and 89 respectively, and for VL FRs the reference sequences are set forth in SEQ ID NO: 122, 123, 125 and 127 respectively.

In any embodiment of any aspect of the invention, the second antigen binding protein comprises an antigen binding domain, wherein the antigen binding domain comprises a VH comprising a complementarity determining region (CDR) 1, CDR2 and CDR3 as defined in any of (e) above and a VL comprising a CDR1, CDR2 and CDR3 as defined in any of (f) above, and:
A. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 115, 116, 118 and 89 respectively, and for VL FRs the reference sequences are set forth in SEQ ID NO: 144, 145, 146 and 97 respectively;
or
B. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 115, 116, 118 and 89 respectively, and for VL FRs the reference sequences are set forth in SEQ ID NO: 161, 162, 163 and 97 respectively;
or
C. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 115, 117, 119 and 89 respectively, and for VL FRs the reference sequences are set forth in SEQ ID NO: 144, 145, 146 and 97 respectively;
or
E. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 115, 117, 119 and 89 respectively, and for VL FRs the reference sequences are set forth in SEQ ID NO: 161, 162, 163 and 97 respectively.

In any embodiment of any aspect of the invention, the second antigen binding protein comprises an antigen binding domain comprising a VH comprising a complementarity determining region (CDR) 1, CDR2 and CDR3 as defined in any of (g) above and a VL comprising a CDR1, CDR2 and CDR3 as defined in any of (h) above, and:
A. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 158, 116, 159 and 89 respectively, and for VL FRs the reference sequences are set forth in SEQ ID NO: 122, 124, 125 and 127 respectively;
or
B. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 158, 116, 159 and 89 respectively, and for VL FRs the reference sequences are set forth in SEQ ID NO: 122, 123, 125 and 127 respectively;
or
C. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 158, 117, 160 and 89 respectively, and for VL FRs the reference sequences are set forth in SEQ ID NO: 122, 124, 125 and 127 respectively;
or
D. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 158, 117, 160 and 89 respectively, and for VL FRs the reference sequences are set forth in SEQ ID NO: 122, 123, 125 and 127 respectively.

In any embodiment of any aspect of the invention, the second antigen binding protein comprises an antigen binding domain, wherein the antigen binding domain comprises a VH comprising a complementarity determining region (CDR) 1, CDR2 and CDR3 as defined in any of (i) above and a VL comprising a CDR1, CDR2 and CDR3 as defined in any of (j) above, and:
A. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 158, 116, 159 and 89 respectively, and for VL FRs the reference sequences are set forth in SEQ ID NO: 144, 145, 146 and 97 respectively;
or
B. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 158, 116, 159 and 89 respectively, and for VL FRs the reference sequences are set forth in SEQ ID NO: 161, 162, 163 and 97 respectively;
or
C. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 158, 117, 160 and 89 respectively, and for VL FRs the reference sequences are set forth in SEQ ID NO: 144, 145, 146 and 97 respectively;
or
D. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 158, 117, 160 and 89 respectively, and for VL FRs the reference sequences are set forth in SEQ ID NO: 161, 162, 163 and 97 respectively.

In any embodiment of any aspect of the invention, the second antigen binding protein comprises an antigen binding domain, wherein the antigen binding domain comprises a VH comprising a complementarity determining region (CDR) 1, CDR2 and CDR3 as defined in any of (k) above and a VL comprising a CDR1, CDR2 and CDR3 as defined in any of (l) above, and:
A. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 128, 130 and 89 respectively, and for VL FRs the reference sequences are set forth in SEQ ID NO: 122, 124, 125 and 97 respectively;
or
B. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 128, 130 and 89 respectively, and for VL FRs the reference sequences are set forth in SEQ ID NO: 122, 123, 125 and 97 respectively;
or
C. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 129, 131 and 89 respectively, and for VL FRs the reference sequences are set forth in SEQ ID NO: 122, 124, 125 and 97 respectively;
or
D. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 129, 131 and 89 respectively, and for VL FRs the reference sequences are set forth in SEQ ID NO: 122, 123, 125 and 97 respectively;
or
E. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 164, 128, 166 and 89 respectively, and for VL FRs the reference sequences are set forth in SEQ ID NO: 122, 124, 125 and 97 respectively;
or
F. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 164, 128, 166 and 89 respectively, and for VL FRs the reference sequences are set forth in SEQ ID NO: 122, 123, 125 and 97 respectively;
or
G. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 164, 165, 167 and 89 respectively, and for VL FRs the reference sequences are set forth in SEQ ID NO: 122, 124, 125 and 97 respectively;
or
H. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NO: 164, 165, 167 and 89 respectively, and for VL FRs the reference sequences are set forth in SEQ ID NO: 122, 123, 125 and 97 respectively.

In any embodiment of any aspect of the invention, the second antigen binding protein comprises an antigen binding domain, wherein the antigen binding domain comprises a VH comprising a complementarity determining region (CDR) 1, CDR2 and CDR3 as defined in any of (m) above and a VL comprising a CDR1, CDR2 and CDR3 as defined in any of (n) above, and:
A. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 128, 130 and 89 respectively, and for VL FRs the reference sequences are set forth in SEQ ID NO: 144, 145, 146 and 97 respectively;
or
B. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 128, 130 and 89 respectively, and for VL FRs the reference sequences are set forth in SEQ ID NO: 168, 169, 170 and 97 respectively;
or
C. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 129, 131 and 89 respectively, and for VL FRs the reference sequences are set forth in SEQ ID NO: 144, 145, 146 and 97 respectively;
or
D. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 129, 131 and 89 respectively, and for VL FRs the reference sequences are set forth in SEQ ID NO: 168, 169, 170 and 97 respectively;
or
E. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 164, 128, 166 and 89 respectively, and for VL FRs the reference sequences are set forth in SEQ ID NO: 144, 145, 146 and 97 respectively;
or
F. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 164, 128, 166 and 89 respectively, and for VL FRs the reference sequences are set forth in SEQ ID NO: 168, 169, 170 and 97 respectively;
or
G. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 164, 165, 167 and 89 respectively, and for VL FRs the reference sequences are set forth in SEQ ID NO: 144, 145, 146 and 97 respectively;
or
H. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 164, 165, 167 and 89 respectively, and for VL FRs the reference sequences are set forth in SEQ ID NO: 168, 169, 170 and 97 respectively.

In any embodiment of any aspect of the invention, the second an antigen binding protein comprises an antigen binding domain, wherein the antigen binding domain comprises
A. a VH comprising a complementarity determining region (CDR) 1, CDR2 and CDR3 as defined in any of (o) above and a VL comprising a CDR1, CDR2 and CDR3 as defined in any of (p) above, and a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 141, 142, 143 and 137 respectively, and for VL FRs the reference sequences are set forth in SEQ ID NO: 144, 145, 146 and 97 respectively;
or
B. a VH comprising a complementarity determining region (CDR) 1, CDR2 and CDR3 as defined in any of (q) above and a VL comprising a CDR1, CDR2 and CDR3 as defined in any of (r) above and a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 147, 148 and 137 respectively, and for VL FRs the reference sequences are set forth in SEQ ID NO: 144, 145, 146 and 97 respectively.

In any embodiment of any aspect of the invention, the second antigen binding protein comprises an antigen binding domain comprising a variable heavy chain comprising the amino acid sequence as set forth in any one of SEQ ID NOs: 71 to 74, or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto. Optionally, the heavy chain variable domain of the second antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEQ ID NOs: 71 to 74; optionally wherein the amino acid substitutions are not in the CDRs and/or the antigen binding protein retains the ability to bind to FLAG tag.

In an embodiment of any aspect of the invention, the second antigen binding protein comprises an antigen binding domain comprising a variable light chain comprising the amino acid sequence as set forth in any one of SEQ ID NOs: 76 to 79; or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto. Optionally, the light chain variable domain of the second antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEQ ID NOs: 76 to 79; optionally wherein the amino acid substitutions are not in the CDRs and/or the antigen binding protein retains the ability to bind to FLAG tag.

In any embodiment of any aspect of the invention, the second antigen binding protein comprises an antigen binding domain comprising a variable heavy chain comprising the amino acid sequence as set forth in any one of SEQ ID NOs: 71 to 74 and a variable light chain comprising the amino acid sequence as set forth in any one of SEQ ID NOs: 76 to 79; or sequences at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto. Optionally, the heavy and light chain variable domains of the second antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEQ ID NOs: 71 to 74 and 76 to 79, respectively; optionally wherein the amino acid substitutions are not in the CDRs and/or the antigen binding protein retains the ability to bind to FLAG tag.

As described herein, the second antigen binding protein may be in the form of:
(i) a single domain antibody (sdAb);
(ii) a single chain Fv fragment (scFv);
(iii) a dimeric scFv (di-scFv);
(iv) one of (ii) or (iii) linked to a constant region of an antibody, Fc or a heavy chain constant domain (CH) 2 and/or CH3;
(v) one of (i) to (iv) linked to a protein that binds to an immune effector cell;
(vi) one of (i) to (iv) linked to a modified immune cell receptor, such as a modified T cell receptor.

Further, as described herein, the second antigen binding protein may be in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody;
(viii) one of (i) to (vii) linked to a constant region of an antibody, Fc or a heavy chain constant domain (CH) 2 and/or CH3.

In preferred embodiments of any aspect of the invention, the second antigen binding protein is in the form of an scFv and the first antigen binding protein is in the form of an immunoglobulin G (IgG) antibody. In alternative embodiments, the second antigen binding protein may be of the same antibody format, or fragment thereof, as the first antigen binding protein. Alternatively, the first and second antigen binding proteins are in different formats of antigen binding protein.

In any embodiment of the first or second aspects of the invention, the second binding domain may be in the form of an antibody or antigen binding fragment thereof. The antigen binding protein may be an antibody, for example, a monoclonal antibody. The antigen binding protein may be in the form of a recombinant or modified antibody (e.g., chimeric antibody, humanised antibody, human antibody, CDR-grafted antibody, primatised antibody, de-immunised antibody, synhumanised antibody, half-antibody, bispecific antibody, trispecific antibody or multispecific antibody). The antibody may further comprise a chemical modification, such as conjugation to an active agent or radiolabel, or an agent for improving solubility or other modification described herein. As used herein the antigen binding protein may be a variable domain.

In any embodiment of any aspect of the invention, the second antigen binding protein comprises, consists essentially of or consists of an amino acid sequence of (in order of N to C terminus or C to N terminus):
SEQ ID NO: 71 and 79;
SEQ ID NO: 71 and 78;
SEQ ID NO: 71 and 76;
SEQ ID NO: 71 and 77;
SEQ ID NO: 72 and 76;
SEQ ID NO: 72 and 77;
SEQ ID NO:72 and 78
SEQ ID NO: 72 and 79;
SEQ ID NO: 73 and 76;
SEQ ID NO: 73 and 77;
SEQ ID NO: 73 and 78;
SEQ ID NO: 73 and 79;
SEQ ID NO: 74 and 76;
SEQ ID NO: 74 and 77;
SEQ ID NO: 74 and 78; or
SEQ ID NO: 74 and 79.

As used herein, the complementarity determining region sequences (CDRs) of an antigen binding protein of the invention are defined according to the IMGT, Chothia or Kabat numbering system or any other CDR numbering system.

In any embodiment of any aspect of the invention, the bispecific polypeptide comprises, consists essentially of or consists an amino acid sequence of:
SEQ ID NO:19 and 20;
SEQ ID NO:19 and 21;
SEQ ID NO: 22 and 23;
SEQ ID NO: 23 and 24;
SEQ ID NO:19 and 25;
SEQ ID NO:19 and 26;
SEQ ID NO: 23 and 27;
SEQ ID NO: 23 and 28;
SEQ ID NO:19 and 29;
SEQ ID NO:19 and 30;
SEQ ID NO:23 and 31;
SEQ ID NO: 23 and 32;
SEQ ID NO: 171, 172 and 173;
SEQ ID NO: 172,174 and 175;
SEQ ID NO: 171, 172 and 176;
SEQ ID NO: 172,174 and 177;
SEQ ID NO:50 and 51;
SEQ ID NO: 50 and 52;
SEQ ID NO: 53 and 54;
SEQ ID NO: 54 and 55;
SEQ ID NO: 50 and 56;
SEQ ID NO: 50 and 57;
SEQ ID NO: 54 and 58;
SEQ ID NO: 54 and 59;
SEQ ID NO: 50 and 60;
SEQ ID NO:50 and 61;
SEQ ID NO: 54 and 62; or
SEQ ID NO: 54 and 63.

In any embodiment of the above, the bispecific polypeptide comprises, consists essentially of or consists of one, two or three polypeptide chains, wherein the one, two or three polypeptide chains are linked by covalent linkers (for example any linker as described herein, including an amino acid(s) or a disulphide bond). The bispecific polypeptide may be heterodimerised, as described herein.

In a third aspect, the present invention provides a fusion protein comprising a first antigen binding protein according to either the first or second aspects of the invention, and a second antigen binding protein as defined according to either the first or second aspects of the invention. The invention also provides a fusion protein comprising a bispecific polypeptide as defined herein according to either the first or second aspects of the invention.

In a fourth aspect, the present invention provides a nucleic acid encoding a bispecific polypeptide of the first or second aspects, or part thereof, of the invention, or a fusion protein of the third aspect. Preferably, the nucleic acid has a nucleotide sequence that encodes any one or more of the amino acid sequences corresponding to SEQ ID NO: 1, 2, 17 to 32, 33, 34, 49 to 63, 64 to 67, 71 to 74, 76 to 79 and 171 to 177.

In any embodiment of the fourth aspect of the invention, the nucleic acid may be DNA (e.g. cDNA) or RNA (e.g. mRNA).

In a fifth aspect, the present invention provides a vector comprising a nucleic acid of the fourth aspect.

In aspects of the invention directed to multiple polypeptide chains that form a bispecific polypeptide or an antigen binding protein, an expression construct comprises a nucleic acid encoding a polypeptide comprising, e.g., a VH operably linked to a promoter and a nucleic acid encoding a polypeptide comprising, e.g., a VL operably linked to a promoter.

In another example, the expression construct is a bicistronic expression construct, e.g., comprising the following operably linked components in 5′ to 3′ order:
(i) a promoter
(ii) a nucleic acid encoding a first polypeptide;
(iii) an internal ribosome entry site; and
(iv) a nucleic acid encoding a second polypeptide,
wherein the first polypeptide comprises a VH and the second polypeptide comprises a VL, or vice versa.

The present invention also contemplates separate expression constructs one of which encodes a first polypeptide comprising a VH and another of which encodes a second polypeptide comprising a VL. For example, the present invention also provides a composition comprising:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter.

In a sixth aspect, the invention provides a cell comprising a vector of the fifth aspect or nucleic acid of the fourth aspect. Preferably, the cell is isolated, substantially purified or recombinant. In one example, the cell comprises an expression construct of the invention or:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter, wherein the first and second polypeptides associate to form an antigen binding protein as described herein.

Examples of cells of the present invention include bacterial cells, yeast cells, insect cells or mammalian cells.

In another embodiment there is provided an animal or tissue derived therefrom comprising a cell described herein.

In a seventh aspect, the present invention provides a pharmaceutical composition comprising a bispecific polypeptide of the first or second aspects, a fusion protein of the third aspect, a nucleic acid of the fourth aspect, a vector of the fifth aspect or a cell of the sixth aspect and a pharmaceutically acceptable carrier, diluent or excipient.

In a eighth aspect, the present invention provides a method for producing a bispecific polypeptide of the first or second aspects, or a fusion protein of the third aspect, comprising expressing a nucleic acid of the fourth aspect in a cell or animal as described herein.

In an ninth aspect, the present invention provides use of a bispecific polypeptide of the first or second aspects, a fusion protein of the third aspect, a nucleic acid of the fourth aspect, a vector of the fifth aspect or a cell of the sixth aspect in the manufacture of a medicament for the treatment or prevention of cancer

In a tenth aspect, the present invention provides a method of treating or preventing cancer, the method comprising administering to a subject a bispecific polypeptide of the first or second aspects, a fusion protein of the third aspect, a nucleic acid of the fourth aspect, a vector of the fifth aspect or a cell of the sixth aspect, thereby treating or preventing cancer.

An antigen binding protein as described herein may be purified, substantially purified, isolated and/or recombinant.

A bispecific polypeptide of the invention may be part of a supernatant taken from media in which a hybridoma expressing a bispecific polypeptide of the invention has been grown.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. ELISA titre of IgG humanised FLAG-tag binding monoclonal antibodies.

FIG. 2: Humanised monoclonal antibodies for binding to FLAG tagged CARs induce IFNgamma secretion from CAR T cells.

FIG. 3: Alignment of VH and VL regions from parental antibody with humanised variants v4 and v7.

FIG. 4. Schematic of BEAT variations for IgG and scFv domains.

FIG. 5. Single referenced sensorgrams of CD40 binding to BEAT1a4 (F7-G8) in the presence of 50 nM FLAG-BAP, showing 50 nM FLAG-BAP binding and saturating followed by CD40 injections in the presence of 50 nM FLAG-BAP. CD40 concentrations 50 nM, 16.7 nM, 5.6 nM, 1.9 nM, 0.62 nM, 0.21 nM, 0.069 nM, 0.023 nM, 0 nM. Fit to 1:1 binding model is shown.

FIG. 6. Binding of anti-CD40 (A) and anti-CD206 (B) BEATs to monocyte derived DCs.

FIG. 7. (A) Dose-dependent binding of BEAT to CD40 on human MoDC cells between 0.1-100 nM BEAT1a4 (B) Dose dependent binding of BEAT to CD40 on human B cells between 0.1-100n M BEAT 1a4 (C) Dose-dependent binding of BEAT to CD40 on non-human primate MoDC cells between 0.1-100 nM BEAT1a4 (D) Dose dependent binding of BEAT to CD40 on on non-human primate B cells between 0.1-100 nM BEAT 1a4.

FIG. 8. BEAT1 (and aCD40 IgG2 control) increased CD86 expression (A) and IL-12/IL-23p40 (B) release in a dose responsive manner between 3-30 nM (CD86) and 1-60 nM (IL-12)

FIG. 9. (A) BEATs bind to FLAG tag on 7B1 anti-mesothelin CD28z CAR and increase IFN-gamma. (B) MoDCs express CD40 and CD206. (C) Evaluation of BEAT1a4 binding to CAR T-cells via the FLAG tag detection via GPF+/AF647+ cells via FACS.

FIG. 10. Cytokine analysis with one donor with BEATs.

FIG. 11. CAR T-Cell proliferation with addition of BEATs in the presence of control compounds/BEAT (30 nM) with or without MoDc after 120 hrs proliferation.

FIG. 12. Further phenotyping of CD4+, CD8+ and CD3+ described in FIG. 11.

FIG. 13. Cytotoxicity of BEAT+CAR T cell in SKOV-3 MSLN positive cell lines.

FIG. 14. In vivo efficacy of CAR T+BEAT1a5 in a SKOV3-Mesothelin positive model transfected with CD40 knock-out cells. (A) Schematic experimental timeline. (B) Survival curves. (C) Tumour size.

FIG. 15. Schematic of BEATs3, 4, 5 and 6.

FIG. 16. ELISA binding assay of anti-CD40 anti-FLAG BEATs 3, 4, 5 and 6 to CD40 antigen.

FIG. 17. Binding of BEATs 1, 3, 4, 5 and 6 to 7B1 anti-mesothelin CD28z CAR human T cells (A) and human monocyte derived DCs (B).

FIG. 18. CD86 (A) and IL-12/IL-23p40 (B) upregulation on MoDCs with the addition of BEATs 1, 3, 4, 5 and 6.

FIG. 19. T-Cell proliferation with addition of BEATs 1, 3, 4, 5 and 6 in the presence of control compounds/BEAT (30 nM) with or without MoDc and CAR T cells after 5 days proliferation.

FIG. 20. Further phenotyping of CD3+ T cells described in FIG. 15.

FIG. 21. Further phenotyping of CD4+ T cells described in FIG. 15.

FIG. 22. Further phenotyping of CD8+ T cells described in FIG. 15.

FIG. 23. Cytokine activity of BEATs 1, 3, 4, 5 and 6 with CAR T cells or MoDCs or CAR+MoDCs including IFN-γ (A), IL12-p70 (B), IL-6 (C), IL-2 (D) MCP-1 (E), TNF-α (F).

FIG. 24. In vivo efficacy of CAR T cells alone compared to CAR T+BEAT2a6 (anti-CD206 BEAT) in a SKOV3-Mesothelin model detailing tumour size.

### DESCRIPTION OF THE SEQUENCES

TABLE 1
Sequences of the invention

| | | |
|---|---|---|
| SEQ ID | | |
| NO | Descriptor | Sequence |

CD40 binding polypeptides

| | | |
|---|---|---|
| 1 | Anti-hCD40 VH | QVQLVQSGAEVKKPGASVKVSCKASGYTFTGYYMHWVRQAPGQGL |
| | | EWMGWINPDSGGTNYAQKFQGRVTMTRDTSISTAYMELNRLRSDD |
| | | TAVYYCARDQPLGYCTNGVCSYFDYWGQGTLVTVSS |
| 2 | Anti-hCD40 VL | DIQMTQSPSSVSASVGDRVTITCRASQGIYSWLAWYQQKPGKAPNL |
| | | LIYTASTLQSGVPSRFSGSGSGTDFTLTISSLQPEDFATYYCQQANIF |
| | | PLTFGGGTKVEIK |
| 3 | Anti-hCD40 CDRH1 | GYYMH |
| | (Kabat) | |
| 4 | Anti-hCD40 CDRH1 | GYTFTGYYMH |
| | (AbM, Other) | |
| 5 | Anti-hCD40 CDRH1 | GYTFTGYY |
| | (IMGT) | |
| 6 | Anti-hCD40 CDRH2 | WINPDSGGTNYAQKFQG |
| | (Kabat) | |
| 7 | Anti-hCD40 CDRH2 | WINPDSGGTN |
| | (AbM) | |
| 8 | Anti-hCD40 CDRH2 | WINPDSGG |
| | (Other) | |
| 9 | Anti-hCD40 CDRH2 | INPDSGGT |
| | (IMGT) | |
| 10 | Anti-hCD40 CDRH3 | DQPLGYCTNGVCSYFDY |
| | (Kabat, AbM, Other) | |
| 11 | Anti-hCD40 CDRH3 | ARDQPLGYCTNGVCSYFDY |
| | (IMGT) | |
| 12 | Anti-hCD40 CDRL1 | RASQGIYSWLA |
| | (Kabat, AbM, Other) | |
| 13 | Anti-hCD40 CDRL1 | QGIYSW |
| | (IMGT) | |
| 14 | Anti-hCD40 CDRL2 | TASTLQS |
| | (Kabat, AbM, Other) | |
| 15 | Anti-hCD40 CDRL2 | TA |
| | (IMGT) | |
| 16 | Anti-hCD40 CDRL3 | QQANIFPLT |
| | (Kabat, AbM, IMGT, | |
| | Other) | |
| 17 | anti-hCD40 IgG2 CH | ASTKGPSVFPLAPCSRSTSESTAALGCLVKDYFPEPVTVSWNSGALT |
| | | SGVHTFPAVLQSSGLYSLSSVVTVPSSNFGTQTYTCNVDHKPSNTKV |
| | | DKTVERKCCVECPPCPAPPVAGPSVFLFPPKPKDTLMISRTPEVTCV |
| | | VVDVSHEDPEVQFNWYVDGVEVHNAKTKPREEQFNSTRFVVSVLTV |
| | | VHQDWLNGKEYKCKVSNKGLPAPIEKTISKTKGQPREPQVYTLPPSR |
| | | EEMTKNQVSLTCLVKGFYPSDIAVEWESNGQPENNYKTTPPMLDSD |
| | | GSFFLYSKLTVDKSRWQQGNVFSCSVMHEALHNHYTQKSLSLSPGK |
| 18 | anti-hCD40 IgG2 & | RTVAAPSVFIFPPSDEQLKSGTASVVCLLNNFYPREAKVQWKVDNAL |
| | anti-hCD206 | QSGNSQESVTEQDSKDSTYSLSSTLTLSKADYEKHKVYACEVTHQG |
| | IgG1LALA CL | LSSPVTKSFNRGEC |
| 19 | BEAT1a1, BEAT1a2, | QVQLVQSGAEVKKPGASVKVSCKASGYTFTGYYMHWVRQAPGQGL |
| | BEAT1a5, BEAT1a6, | EWMGWINPDSGGTNYAQKFQGRVTMTRDTSISTAYMELNRLRSDD |
| | BEAT1b1, BEAT1b2 | TAVYYCARDQPLGYCTNGVCSYFDYWGQGTLVTVSSASTKGPSVFP |
| | HC-IgG2 (anti-CD40) | LAPCSRSTSESTAALGCLVKDYFPEPVTVSWNSGALTSGVHTFPAVL |
| | | QSSGLYSLSSVVTVPSSNFGTQTYTCNVDHKPSNTKVDKTVERKCC |
| | | VECPPCPAPPVAGPSVFLFPPKPKDTLMISRTPEVTCVVVDVSHEDP |
| | | EVQFNWYVDGVEVHNAKTKPREEQFNSTFRVVSVLTVVHQDWLNG |
| | | KEYKCKVSNKGLPAPIEKTISKTKGQPREPQVYTLPPSREEMTKNQV |
| | | SLTCLVKGFYPSDIAVEWESNGQPENNYKTTPPMLDSDGSFFLYSKL |
| | | TVDKSRWQQGNVFSCSVMHEALHNHYTQKSLSLSPGK |
| 20 | BEAT1a1 | DVVMTQSPDSLAVSLGERATINCKSSQSIVHRNGNTYLEWYQQKPG |
| | hM2V4-Clinker- | QPPKLLIYKVSNRFSGVPDRFSGSGSGTDFTLTISSLQAEDVAVYYCF |
| | (GGGGS)3-LC-IgG2 | QGSHVPYTFGGGTKLEIKGGGGSGGGGSGGGGSQVQLVQSGAEV |
| | (anti-CD40) | KKPGASVKVSCKASGYSFTTYTIHWVRQAPGQGLEWIGYINPSSGYA |
| | | AYNQKFQGRVTLTADKSTSTAYMELSSLRSEDTAVYYCAREKFYGY |
| | | DYWGQGTTVTVSSGGGGSGGGGSGGGGSDIQMTQSPSSVSASVG |
| | | DRVTITCRASQGIYSWLAWYQQKPGKAPNLLIYTASTLQSGVPSRFS |
| | | GSGSGTDFTLTISSLQPEDFATYYCQQANIFPLTFGGGTKVEIKRTVA |
| | | APSVFIFPPSDEQLKSGTASVVCLLNNFYPREAKVQWKVDNALQSG |
| | | NSQESVTEQDSKDSTYSLSSTLTLSKADYEKHKVYACEVTHQGLSSP |
| | | VTKSFNRGEC |
| 21 | BEAT1a2 | DIQMTQSPSSVSASVGDRVTITCRASQGIYSWLAWYQQKPGKAPNL |
| | 3-hM2V4-Clinker- | LIYTASTLQSGVPSRFSGSGSGTDFTLTISSLQPEDFATYYCQQANIF |
| | (GGGGS)LC-IgG2 | PLTFGGGTKVEIKRTVAAPSVFIFPPSDEQLKSGTASVVCLLNNFYPR |
| | (anti-CD40) | EAKVQWKVDNALQSGNSQESVTEQDSKDSTYSLSSTLTLSKADYEK |
| | | HKVYACEVTHQGLSSPVTKSFNRGECGGGGSGGGGSGGGGSDVV |
| | | MTQSPDSLAVSLGERATINCKSSQSIVHRNGNTYLEWYQQKPGQPP |
| | | KLLIYKVSNRFSGVPDRFSGSGSGTDFTLTISSLQAEDVAVYYCFQG |
| | | SHVPYTFGGGTKLEIKGGGGSGGGGSGGGGSQVQLVQSGAEVKKP |
| | | GASVKVSCKASGYSFTTYTIHWVRQAPGQGLEWIGYINPSSGYAAY |
| | | NQKFQGRVTLTADKSTSTAYMELSSLRSEDTAVYYCAREKFYGYDY |
| | | WGQGTTVTVSS |
| 22 | BEAT1a3 | DVVMTQSPDSLAVSLGERATINCKSSQSIVHRNGNTYLEWYQQKPG |
| | hM2V4_Clinker- | QPPKLLIYKVSNRFSGVPDRFSGSGSGTDFTLTISSLQAEDVAVYYCF |
| | (GGGGS) | QGSHVPYTFGGGTKLEIKGGGGSGGGGSGGGGSQVQLVQSGAEV |
| | 3-HC-IgG2 (anti- | KKPGASVKVSCKASGYSFTTYTIHWVRQAPGQGLEWIGYINPSSGYA |
| | CD40) | AYNQKFQGRVTLTADKSTSTAYMELSSLRSEDTAVYYCAREKFYGY |
| | | DYWGQGTTVTVSSGGGGSGGGGSGGGGSQVQLVQSGAEVKKPG |
| | | ASVKVSCKASGYTFTGYYMHWVRQAPGQGLEWMGWINPDSGGTN |
| | | YAQKFQGRVTMTRDTSISTAYMELNRLRSDDTAVYYCARDQPLGYC |
| | | TNGVCSYFDYWGQGTLVTVSSASTKGPSVFPLAPCSRSTSESTAAL |
| | | GCLVKDYFPEPVTVSWNSGALTSGVHTFPAVLQSSGLYSLSSVVTVP |
| | | SSNFGTQTYTCNVDHKPSNTKVDKTVERKCCVECPPCPAPPVAGPS |
| | | VFLFPPKPKDTLMISRTPEVTCVVVDVSHEDPEVQFNWYVDGVEVH |
| | | NAKTKPREEQFNSTFRVVSVLTVVHQDWLNGKEYKCKVSNKGLPAP |
| | | IEKTISKTKGQPREPQVYTLPPSREEMTKNQVSLTCLVKGFYPSDIAV |
| | | EWESNGQPENNYKTTPPMLDSDGSFFLYSKLTVDKSRWQQGNVFS |
| | | CSVMHEALHNHYTQKSLSLSPGK |
| 23 | BEAT1a3, BEAT1a4, | DIQMTQSPSSVSASVGDRVTITCRASQGIYSWLAWYQQKPGKAPNL |
| | BEAT1a7, BEAT1a8, | LIYTASTLQSGVPSRFSGSGSGTDFTLTISSLQPEDFATYYCQQANIF |
| | BEAT1b3, BEAT1b4 | PLTFGGGTKVEIKRTVAAPSVFIFPPSDEQLKSGTASVVCLLNNFYPR |
| | LC-IgG2 (anti-CD40) | EAKVQWKVDNALQSGNSQESVTEQDSKDSTYSLSSTLTLSKADYEK |
| | | HKVYACEVTHQGLSSPVTKSFNRGEC |
| 24 | BEAT1a4 | QVQLVQSGAEVKKPGASVKVSCKASGYTFTGYYMHWVRQAPGQGL |
| | HC-IgG2(anti-CD40)- | EWMGWINPDSGGTNYAQKFQGRVTMTRDTSISTAYMELNRLRSDD |
| | (GGGGS)3- | TAVYYCARDQPLGYCTNGVCSYFDYWGQGTLVTVSSASTKGPSVFP |
| | hM2V4_Clinker | LAPCSRSTSESTAALGCLVKDYFPEPVTVSWNSGALTSGVHTFPAVL |
| | | QSSGLYSLSSVVTVPSSNFGTQTYTCNVDHKPSNTKVDKTVERKCC |
| | | VECPPCPAPPVAGPSVFLFPPKPKDTLMISRTPEVTCVVVDVSHEDP |
| | | EVQFNWYVDGVEVHNAKTKPREEQFNSTFRVVSVLTVVHQDWLNG |
| | | KEYKCKVSNKGLPAPIEKTISKTKGQPREPQVYTLPPSREEMTKNQV |
| | | SLTCLVKGFYPSDIAVEWESNGQPENNYKTTPPMLDSDGSFFLYSKL |
| | | TVDKSRWQQGNVFSCSVMHEALHNHYTQKSLSLSPGKGGGGSGG |
| | | GGSGGGGSDVVMTQSPDSLAVSLGERATINCKSSQSIVHRNGNTYL |
| | | EWYQQKPGQPPKLLIYKVSNRFSGVPDRFSGSGSGTDFTLTISSLQA |
| | | EDVAVYYCFQGSHVPYTFGGGTKLEIKGGGGSGGGGSGGGGSQV |
| | | QLVQSGAEVKKPGASVKVSCKASGYSFTTYTIHWVRQAPGQGLEWI |
| | | GYINPSSGYAAYNQKFQGRVTLTADKSTSTAYMELSSLRSEDTAVYY |
| | | CAREKFYGYDYWGQGTTVTVSS |
| 25 | BEAT1a5 | QVQLVQSGAEVKKPGASVKVSCKASGYSFTTYTIHWVRQAPGQGLE |
| | hM2V4_PTGXLinker- | WIGYINPSSGYAAYNQKFQGRVTLTADKSTSTAYMELSSLRSEDTAV |
| | (GGGGS)3-LC- | YYCAREKFYGYDYWGQGTTVTVSSGGGGSGGGGSGGGGSGGGG |
| | IgG2(anti-CD40) | ASDVVMTQSPDSLAVSLGERATINCKSSQSIVHRNGNTYLEWYQQK |
| | | PGQPPKLLIYKVSNRFSGVPDRFSGSGSGTDFTLTISSLQAEDVAVY |
| | | YCFQGSHVPYTFGGGTKLEIKGGGGSGGGGSGGGGSDIQMTQSPS |
| | | SVSASVGDRVTITCRASQGIYSWLAWYQQKPGKAPNLLIYTASTLQS |
| | | GVPSRFSGSGSGTDFTLTISSLQPEDFATYYCQQANIFPLTFGGGTK |
| | | VEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVT |
| | | HQGLSSPVTKSFNRGEC |
| 26 | BEAT1a6 | DIQMTQSPSSVSASVGDRVTITCRASQGIYSWLAWYQQKPGKAPNL |
| | LC-IgG2(anti-CD40)- | LIYTASTLQSGVPSRFSGSGSGTDFTLTISSLQPEDFATYYCQQANIF |
| | (GGGGS)3- | PLTFGGGTKVEIKRTVAAPSVFIFPPSDEQLKSGTASVVCLLNNFYPR |
| | hM2V4_PTGXLinker | EAKVQWKVDNALQSGNSQESVTEQDSKDSTYSLSSTLTLSKADYEK |
| | | HKVYACEVTHQGLSSPVTKSFNRGECGGGGSGGGGSGGGGSQVQ |
| | | LVQSGAEVKKPGASVKVSCKASGYSFTTYTIHWVRQAPGQGLEWIG |
| | | YINPSSGYAAYNQKFQGRVTLTADKSTSTAYMELSSLRSEDTAVYYC |
| | | AREKFYGYDYWGQGTTVTVSSGGGGSGGGGSGGGGSGGGGASD |
| | | VVMTQSPDSLAVSLGERATINCKSSQSIVHRNGNTYLEWYQQKPGQ |
| | | PPKLLIYKVSNRFSGVPDRFSGSGSGTDFTLTISSLQAEDVAVYYCFQ |
| | | GSHVPYTFGGGTKLEIK |
| 27 | BEAT1a7 | QVQLVQSGAEVKKPGASVKVSCKASGYSFTTYTIHWVRQAPGQGLE |
| | hM2V4_PTGXLinker- | WIGYINPSSGYAAYNQKFQGRVTLTADKSTSTAYMELSSLRSEDTAV |
| | (GGGGS)3-HC- | YYCAREKFYGYDYWGQGTTVTVSSGGGGSGGGGSGGGGSGGGG |
| | IgG2(anti-CD40) | ASDVVMTQSPDSLAVSLGERATINCKSSQSIVHRNGNTYLEWYQQK |
| | | PGQPPKLLIYKVSNRFSGVPDRFSGSGSGTDFTLTISSLQAEDVAVY |
| | | YCFQGSHVPYTFGGGTKLEIKGGGGSGGGGSGGGGSQVQLVQSG |
| | | AEVKKPGASVKVSCKASGYTFTGYYMHWVRQAPGQGLEWMGWIN |
| | | PDSGGTNYAQKFQGRVTMTRDTSISTAYMELNRLRSDDTAVYYCAR |
| | | DQPLGYCTNGVCSYFDYWGQGTLVTVSSASTKGPSVFPLAPCSRST |
| | | SESTAALGCLVKDYFPEPVTVSWNSGALTSGVHTFPAVLQSSGLYSL |
| | | SSVVTVPSSNFGTQTYTCNVDHKPSNTKVDKTVERKCCVECPPCPA |
| | | PPVAGPSVFLFPPKPKDTLMISRTPEVTCVVVDVSHEDPEVQFNWYV |
| | | DGVEVHNAKTKPREEQFNSTFRVVSVLTVVHQDWLNGKEYKCKVSN |
| | | KGLPAPIEKTISKTKGQPREPQVYTLPPSREEMTKNQVSLTCLVKGFY |
| | | PSDIAVEWESNGQPENNYKTTPPMLDSDGSFFLYSKLTVDKSRWQQ |
| | | GNVFSCSVMHEALHNHYTQKSLSLSPGK |
| 28 | BEAT1a8 | QVQLVQSGAEVKKPGASVKVSCKASGYTFTGYYMHWVRQAPGQGL |
| | HC-IgG2(anti-CD40)- | EWMGWINPDSGGTNYAQKFQGRVTMTRDTSISTAYMELNRLRSDD |
| | (GGGGS)3- | TAVYYCARDQPLGYCTNGVCSYFDYWGQGTLVTVSSASTKGPSVFP |
| | hM2V4_PTGXLinker | LAPCSRSTSESTAALGCLVKDYFPEPVTVSWNSGALTSGVHTFPAVL |
| | | QSSGLYSLSSVVTVPSSNFGTQTYTCNVDHKPSNTKVDKTVERKCC |
| | | VECPPCPAPPVAGPSVFLFPPKPKDTLMISRTPEVTCVVVDVSHEDP |
| | | EVQFNWYVDGVEVHNAKTKPREEQFNSTFRVVSVLTVVHQDWLNG |
| | | KEYKCKVSNKGLPAPIEKTISKTKGQPREPQVYTLPPSREEMTKNQV |
| | | SLTCLVKGFYPSDIAVEWESNGQPENNYKTTPPMLDSDGSFFLYSKL |
| | | TVDKSRWQQGNVFSCSVMHEALHNHYTQKSLSLSPGKGGGGSGG |
| | | GGSGGGGSQVQLVQSGAEVKKPGASVKVSCKASGYSFTTYTIHWV |
| | | RQAPGQGLEWIGYINPSSGYAAYNQKFQGRVTLTADKSTSTAYMEL |
| | | SSLRSEDTAVYYCAREKFYGYDYWGQGTTVTVSSGGGGSGGGGS |
| | | GGGGSGGGGASDVVMTQSPDSLAVSLGERATINCKSSQSIVHRNG |
| | | NTYLEWYQQKPGQPPKLLIYKVSNRFSGVPDRFSGSGSGTDFTLTIS |
| | | SLQAEDVAVYYCFQGSHVPYTFGGGTKLEIK |
| 29 | BEAT1b1 | DVVMTQSPDSLAVSLGERATINCKSSQSIVHRNGNTYLEWYQLKPG |
| | hM2V7_CLinker- | QPPKLLIYKVSNRFSGVPDRFSGSGSGTDFTLTISSLQAEDVAVYYCF |
| | (GGGGS)3- LC- | QGSHVPYTFGGGTKLEIKGGGGSGGGGSGGGGSQVQLVQSGAEV |
| | IgG2(anti-CD40) | KKPGASVKVSCKASGYSFTTYTMHWVRQAPGQGLEWMGYINPSSG |
| | | YAAYNQKFQGRVTMTADKSTSTAYMELSSLRSEDTAVYYCAREKFY |
| | | GYDYWGQGTTVTVSSGGGGSGGGGSGGGGSDIQMTQSPSSVSAS |
| | | VGDRVTITCRASQGIYSWLAWYQQKPGKAPNLLIYTASTLQSGVPSR |
| | | FSGSGSGTDFTLTISSLQPEDFATYYCQQANIFPLTFGGGTKVEIKRT |
| | | VAAPSVFIFPPSDEQLKSGTASVVCLLNNFYPREAKVQWKVDNALQS |
| | | GNSQESVTEQDSKDSTYSLSSTLTLSKADYEKHKVYACEVTHQGLS |
| | | SPVTKSFNRGEC |
| 30 | BEAT1b2 | DIQMTQSPSSVSASVGDRVTITCRASQGIYSWLAWYQQKPGKAPNL |
| | LC-IgG2(anti-CD40)- | LIYTASTLQSGVPSRFSGSGSGTDFTLTISSLQPEDFATYYCQQANIF |
| | (GGGGS)3- | PLTFGGGTKVEIKRTVAAPSVFIFPPSDEQLKSGTASVVCLLNNFYPR |
| | hM2V7_Clinker | EAKVQWKVDNALQSGNSQESVTEQDSKDSTYSLSSTLTLSKADYEK |
| | | HKVYACEVTHQGLSSPVTKSFNRGECGGGGSGGGGSGGGGSDVV |
| | | MTQSPDSLAVSLGERATINCKSSQSIVHRNGNTYLEWYQLKPGQPP |
| | | KLLIYKVSNRFSGVPDRFSGSGSGTDFTLTISSLQAEDVAVYYCFQG |
| | | SHVPYTFGGGTKLEIKGGGGSGGGGSGGGGSQVQLVQSGAEVKKP |
| | | GASVKVSCKASGYSFTTYTMHWVRQAPGQGLEWMGYINPSSGYAA |
| | | YNQKFQGRVTMTADKSTSTAYMELSSLRSEDTAVYYCAREKFYGYD |
| | | YWGQGTTVTVSS |
| 31 | BEAT1b3 | DVVMTQSPDSLAVSLGERATINCKSSQSIVHRNGNTYLEWYQLKPG |
| | hM2V7_CLinker- | QPPKLLIYKVSNRFSGVPDRFSGSGSGTDFTLTISSLQAEDVAVYYCF |
| | (GGGGS)3- HC- | QGSHVPYTFGGGTKLEIKGGGGSGGGGSGGGGSQVQLVQSGAEV |
| | IgG2(anti-CD40) | KKPGASVKVSCKASGYSFTTYTMHWVRQAPGQGLEWMGYINPSSG |
| | | YAAYNQKFQGRVTMTADKSTSTAYMELSSLRSEDTAVYYCAREKFY |
| | | GYDYWGQGTTVTVSSGGGGSGGGGSGGGGSQVQLVQSGAEVKK |
| | | PGASVKVSCKASGYTFTGYYMHWVRQAPGQGLEWMGWINPDSGG |
| | | TNYAQKFQGRVTMTRDTSISTAYMELNRLRSDDTAVYYCARDQPLG |
| | | YCTNGVCSYFDYWGQGTLVTVSSASTKGPSVFPLAPCSRSTSESTA |
| | | ALGCLVKDYFPEPVTVSWNSGALTSGVHTFPAVLQSSGLYSLSSVVT |
| | | VPSSNFGTQTYTCNVDHKPSNTKVDKTVERKCCVECPPCPAPPVAG |
| | | PSVFLFPPKPKDTLMISRTPEVTCVVVDVSHEDPEVQFNWYVDGVEV |
| | | HNAKTKPREEQFNSTFRVVSVLTVVHQDWLNGKEYKCKVSNKGLPA |
| | | PIEKTISKTKGQPREPQVYTLPPSREEMTKNQVSLTCLVKGFYPSDIA |
| | | VEWESNGQPENNYKTTPPMLDSDGSFFLYSKLTVDKSRWQQGNVF |
| | | SCSVMHEALHNHYTQKSLSLSPGK |
| 32 | BEAT1b4 | QVQLVQSGAEVKKPGASVKVSCKASGYTFTGYYMHWVRQAPGQGL |
| | HC-IgG2(anti-CD40)- | EWMGWINPDSGGTNYAQKFQGRVTMTRDTSISTAYMELNRLRSDD |
| | (GGGGS)3- | TAVYYCARDQPLGYCTNGVCSYFDYWGQGTLVTVSSASTKGPSVFP |
| | hM2V7_Clinker | LAPCSRSTSESTAALGCLVKDYFPEPVTVSWNSGALTSGVHTFPAVL |
| | | QSSGLYSLSSVVTVPSSNFGTQTYTCNVDHKPSNTKVDKTVERKCC |
| | | VECPPCPAPPVAGPSVFLFPPKPKDTLMISRTPEVTCVVVDVSHEDP |
| | | EVQFNWYVDGVEVHNAKTKPREEQFNSTFRVVSVLTVVHQDWLNG |
| | | KEYKCKVSNKGLPAPIEKTISKTKGQPREPQVYTLPPSREEMTKNQV |
| | | SLTCLVKGFYPSDIAVEWESNGQPENNYKTTPPMLDSDGSFFLYSKL |
| | | TVDKSRWQQGNVFSCSVMHEALHNHYTQKSLSLSPGKGGGGSGG |
| | | GGSGGGGSDVVMTQSPDSLAVSLGERATINCKSSQSIVHRNGNTYL |
| | | EWYQLKPGQPPKLLIYKVSNRFSGVPDRFSGSGSGTDFTLTISSLQA |
| | | EDVAVYYCFQGSHVPYTFGGGTKLEIKGGGGSGGGGSGGGGSQV |
| | | QLVQSGAEVKKPGASVKVSCKASGYSFTTYTMHWVRQAPGQGLEW |
| | | MGYINPSSGYAAYNQKFQGRVTMTADKSTSTAYMELSSLRSEDTAV |
| | | YYCAREKFYGYDYWGQGTTVTVSS |

CD206 binding polypeptides

| | | |
|---|---|---|
| 33 | Anti-hCD206 VH | EVQLVQSGAEVKKPGESLRISCKGSGDSFTTYWIGWVRQMPGKGLE |
| | | WMGIIYPGDSDTIYSPSFQGQVTISADKSISTAYLQWSSLKASDTAMY |
| | | YCTRGDRGVDYWGQGTLVTVSS |
| 34 | Anti-hCD206 VL | DIQMTQSPSSLSASVGDRVTITCRASQGISRWLAWYQQKPEKAPKSL |
| | | IYAASSLQSGVPSRFSGSGSGTDFTLTISGLQPEDFATYYCQQYNSY |
| | | PRTFGQGTKVEIK |
| 35 | Anti-hCD206 CDRH1 | TYWIG |
| | (Kabat) | |
| 36 | Anti-hCD206 CDRH1 | GDSFTTYWIG |
| | (AbM, Other) | |
| 37 | Anti-hCD206 CDRH1 | GDSFTTYW |
| | (IMGT) | |
| 38 | Anti-hCD206 CDRH2 | IIYPGDSDTIYSPSFQG |
| | (Kabat) | |
| 39 | Anti-hCD206 CDRH2 | IIYPGDSDTI |
| | (AbM) | |
| 40 | Anti-hCD206 CDRH2 | IIYPGDS |
| | (Other) | |
| 41 | Anti-hCD206 CDRH2 | IYPGDSDT |
| | (IMGT) | |
| 42 | Anti-hCD206 CDRH3 | GDRGVDY |
| | (Kabat, AbM, Other) | |
| 43 | Anti-hCD206 CDRH3 | TRGDRGVDY |
| | (IMGT) | |
| 44 | Anti-hCD206 CDRL1 | RASQGISRWLA |
| | (Kabat, AbM, Other) | |
| 45 | Anti-hCD206 CDRL1 | QGISRW |
| | (IMGT) | |
| 46 | Anti-hCD206 CDRL2 | AASSLQS |
| | (Kabat, AbM, Other) | |
| 47 | Anti-hCD206 CDRL2 | AA |
| | (IMGT) | |
| 48 | Anti-hCD206 CDRL3 | QQYNSYPRT |
| | (Kabat, AbM, IMGT, | |
| | Other) | |
| 49 | Anti-hCD206 | ASTKGPSVFPLAPSSKSTSGGTAALGCLVKDYFPEPVTVSWNSGALT |
| | IgG1LALA CH | SGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRTPE |
| | | VTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPP |
| | | VLDSDGSFFLYSKLTVDKSRWQQGNVFSCSVMHEALHNHYTQKSLS |
| | | LSPGK |
| 50 | BEAT2a1, BEAT2a2, | EVQLVQSGAEVKKPGESLRISCKGSGDSFTTYWIGWVRQMPGKGLE |
| | BEAT2a5, BEAT2a6, | WMGIIYPGDSDTIYSPSFQGQVTISADKSISTAYLQWSSLKASDTAMY |
| | BEAT2b1, BEAT2b2 | YCTRGDRGVDYWGQGTLVTVSSASTKGPSVFPLAPSSKSTSGGTAA |
| | HC-IgG1LALA(anti- | LGCLVKDYFPEPVTVSWNSGALTSGVHTFPAVLQSSGLYSLSSVVTV |
| | CD206)- | PSSSLGTQTYICNVNHKPSNTKVDKKVEPKSCDKTHTCPPCPAPEAA |
| | G1m17allotype | GGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNKA |
| | | LPAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGN |
| | | VFSCSVMHEALHNHYTQKSLSLSPGK |
| 51 | BEAT2a1 | DVVMTQSPDSLAVSLGERATINCKSSQSIVHRNGNTYLEWYQQKPG |
| | hM2V4_CLinker- | QPPKLLIYKVSNRFSGVPDRFSGSGSGTDFTLTISSLQAEDVAVYYCF |
| | (GGGGS)3-LC- | QGSHVPYTFGGGTKLEIKGGGGSGGGGSGGGGSQVQLVQSGAEV |
| | IgG1LALA(anti- | KKPGASVKVSCKASGYSFTTYTIHWVRQAPGQGLEWIGYINPSSGYA |
| | CD206) | AYNQKFQGRVTLTADKSTSTAYMELSSLRSEDTAVYYCAREKFYGY |
| | | DYWGQGTTVTVSSGGGGSGGGGSGGGGSDIQMTQSPSSLSASVG |
| | | DRVTITCRASQGISRWLAWYQQKPEKAPKSLIYAASSLQSGVPSRFS |
| | | GSGSGTDFTLTISGLQPEDFATYYCQQYNSYPRTFGQGTKVEIKRTV |
| | | AAPSVFIFPPSDEQLKSGTASVVCLLNNFYPREAKVQWKVDNALQSG |
| | | NSQESVTEQDSKDSTYSLSSTLTLSKADYEKHKVYACEVTHQGLSSP |
| | | VTKSFNRGEC |
| 52 | BEAT2a2 | DIQMTQSPSSLSASVGDRVTITCRASQGISRWLAWYQQKPEKAPKSL |
| | LC-IgG1LALA(anti- | IYAASSLQSGVPSRFSGSGSGTDFTLTISGLQPEDFATYYCQQYNSY |
| | CD206)-(GGGGS)3- | PRTFGQGTKVEIKRTVAAPSVFIFPPSDEQLKSGTASVVCLLNNFYPR |
| | hM2V4_Clinker | EAKVQWKVDNALQSGNSQESVTEQDSKDSTYSLSSTLTLSKADYEK |
| | | HKVYACEVTHQGLSSPVTKSFNRGECGGGGSGGGGSGGGGSDVV |
| | | MTQSPDSLAVSLGERATINCKSSQSIVHRNGNTYLEWYQQKPGQPP |
| | | KLLIYKVSNRFSGVPDRFSGSGSGTDFTLTISSLQAEDVAVYYCFQG |
| | | SHVPYTFGGGTKLEIKGGGGSGGGGSGGGGSQVQLVQSGAEVKKP |
| | | GASVKVSCKASGYSFTTYTIHWVRQAPGQGLEWIGYINPSSGYAAY |
| | | NQKFQGRVTLTADKSTSTAYMELSSLRSEDTAVYYCAREKFYGYDY |
| | | WGQGTTVTVSS |
| 53 | BEAT2a3 | DVVMTQSPDSLAVSLGERATINCKSSQSIVHRNGNTYLEWYQQKPG |
| | hM2V4_CLinker- | QPPKLLIYKVSNRFSGVPDRFSGSGSGTDFTLTISSLQAEDVAVYYCF |
| | (GGGGS)3- HC- | QGSHVPYTFGGGTKLEIKGGGGSGGGGSGGGGSQVQLVQSGAEV |
| | IgG1LALA(anti- | KKPGASVKVSCKASGYSFTTYTIHWVRQAPGQGLEWIGYINPSSGYA |
| | CD206) | AYNQKFQGRVTLTADKSTSTAYMELSSLRSEDTAVYYCAREKFYGY |
| | | DYWGQGTTVTVSSGGGGSGGGGSGGGGSEVQLVQSGAEVKKPGE |
| | | SLRISCKGSGDSFTTYWIGWVRQMPGKGLEWMGIIYPGDSDTIYSPS |
| | | FQGQVTISADKSISTAYLQWSSLKASDTAMYYCTRGDRGVDYWGQG |
| | | TLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNH |
| | | KPSNTKVDKKVEPKSCDKTHTCPPCPAPEAAGGPSVFLFPPKPKDTL |
| | | MISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHY |
| | | TQKSLSLSPGK |
| 54 | BEAT2a3, BEAT2a4, | DIQMTQSPSSLSASVGDRVTITCRASQGISRWLAWYQQKPEKAPKSL |
| | BEAT2a7, BEAT2a8, | IYAASSLQSGVPSRFSGSGSGTDFTLTISGLQPEDFATYYCQQYNSY |
| | BEAT2b3, BEAT2b4 | PRTFGQGTKVEIKRTVAAPSVFIFPPSDEQLKSGTASVVCLLNNFYPR |
| | LC-IgG1LALA(anti- | EAKVQWKVDNALQSGNSQESVTEQDSKDSTYSLSSTLTLSKADYEK |
| | CD206) | HKVYACEVTHQGLSSPVTKSFNRGEC |
| 55 | BEAT2a4 | EVQLVQSGAEVKKPGESLRISCKGSGDSFTTYWIGWVRQMPGKGLE |
| | HC-IgG1LALA(anti- | WMGIIYPGDSDTIYSPSFQGQVTISADKSISTAYLQWSSLKASDTAMY |
| | CD206)-(GGGGS)3- | YCTRGDRGVDYWGQGTLVTVSSASTKGPSVFPLAPSSKSTSGGTAA |
| | hM2V4_Clinker | LGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPEAA |
| | | GGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNKA |
| | | LPAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGN |
| | | VFSCSVMHEALHNHYTQKSLSLSPGKGGGGSGGGGSGGGGSDVV |
| | | MTQSPDSLAVSLGERATINCKSSQSIVHRNGNTYLEWYQQKPGQPP |
| | | KLLIYKVSNRFSGVPDRFSGSGSGTDFTLTISSLQAEDVAVYYCFQG |
| | | SHVPYTFGGGTKLEIKGGGGSGGGGSGGGGSQVQLVQSGAEVKKP |
| | | GASVKVSCKASGYSFTTYTIHWVRQAPGQGLEWIGYINPSSGYAAY |
| | | NQKFQGRVTLTADKSTSTAYMELSSLRSEDTAVYYCAREKFYGYDY |
| | | WGQGTTVTVSS |
| 56 | BEAT2a5 | QVQLVQSGAEVKKPGASVKVSCKASGYSFTTYTIHWVRQAPGQGLE |
| | hM2V4_PTGXLinker- | WIGYINPSSGYAAYNQKFQGRVTLTADKSTSTAYMELSSLRSEDTAV |
| | (GGGGS)3-LC- | YYCAREKFYGYDYWGQGTTVTVSSGGGGSGGGGSGGGGSGGGG |
| | IgG1LALA(anti- | ASDVVMTQSPDSLAVSLGERATINCKSSQSIVHRNGNTYLEWYQQK |
| | CD206) | PGQPPKLLIYKVSNRFSGVPDRFSGSGSGTDFTLTISSLQAEDVAVY |
| | | YCFQGSHVPYTFGGGTKLEIKGGGGSGGGGSGGGGSDIQMTQSPS |
| | | SLSASVGDRVTITCRASQGISRWLAWYQQKPEKAPKSLIYAASSLQS |
| | | GVPSRFSGSGSGTDFTLTISGLQPEDFATYYCQQYNSYPRTFGQGT |
| | | KVEIKRTVAAPSVFIFPPSDEQLKSGTASVVCLLNNFYPREAKVQWK |
| | | VDNALQSGNSQESVTEQDSKDSTYSLSSTLTLSKADYEKHKVYACE |
| | | VTHQGLSSPVTKSFNRGEC |
| 57 | BEAT2a6 | DIQMTQSPSSLSASVGDRVTITCRASQGISRWLAWYQQKPEKAPKSL |
| | LC-IgG1LALA(anti- | IYAASSLQSGVPSRFSGSGSGTDFTLTISGLQPEDFATYYCQQYNSY |
| | CD206)-(GGGGS)3- | PRTFGQGTKVEIKRTVAAPSVFIFPPSDEQLKSGTASVVCLLNNFYPR |
| | hM2V4_PTGXLinker | EAKVQWKVDNALQSGNSQESVTEQDSKDSTYSLSSTLTLSKADYEK |
| | | HKVYACEVTHQGLSSPVTKSFNRGECGGGGSGGGGSGGGGSQVQ |
| | | LVQSGAEVKKPGASVKVSCKASGYSFTTYTIHWVRQAPGQGLEWIG |
| | | YINPSSGYAAYNQKFQGRVTLTADKSTSTAYMELSSLRSEDTAVYYC |
| | | AREKFYGYDYWGQGTTVTVSSGGGGSGGGGSGGGGSGGGGASD |
| | | VVMTQSPDSLAVSLGERATINCKSSQSIVHRNGNTYLEWYQQKPGQ |
| | | PPKLLIYKVSNRFSGVPDRFSGSGSGTDFTLTISSLQAEDVAVYYCFQ |
| | | GSHVPYTFGGGTKLEIK |
| 58 | BEAT2a7 | QVQLVQSGAEVKKPGASVKVSCKASGYSFTTYTIHWVRQAPGQGLE |
| | hM2V4_PTGXLinker- | WIGYINPSSGYAAYNQKFQGRVTLTADKSTSTAYMELSSLRSEDTAV |
| | (GGGGS)3- HC- | YYCAREKFYGYDYWGQGTTVTVSSGGGGSGGGGSGGGGSGGGG |
| | IgG1LALA(anti- | ASDVVMTQSPDSLAVSLGERATINCKSSQSIVHRNGNTYLEWYQQK |
| | CD206) | PGQPPKLLIYKVSNRFSGVPDRFSGSGSGTDFTLTISSLQAEDVAVY |
| | | YCFQGSHVPYTFGGGTKLEIKGGGGSGGGGSGGGGSEVQLVQSGA |
| | | EVKKPGESLRISCKGSGDSFTTYWIGWVRQMPGKGLEWMGIIYPGD |
| | | SDTIYSPSFQGQVTISADKSISTAYLQWSSLKASDTAMYYCTRGDRG |
| | | VDYWGQGTLVTVSSASTKGPSVFPLAPSSKSTSGGTAALGCLVKDY |
| | | FPEPVTVSWNSGALTSGVHTFPAVLQSSGLYSLSSVVTVPSSSLGTQ |
| | | TYICNVNHKPSNTKVDKKVEPKSCDKTHTCPPCPAPEAAGGPSVFLF |
| | | PPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTI |
| | | SKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSV |
| | | MHEALHNHYTQKSLSLSPGK |
| 59 | BEAT2a8 | EVQLVQSGAEVKKPGESLRISCKGSGDSFTTYWIGWVRQMPGKGLE |
| | HC-IgG1LALA(anti- | WMGIIYPGDSDTIYSPSFQGQVTISADKSISTAYLQWSSLKASDTAMY |
| | CD206)-(GGGGS)3- | YCTRGDRGVDYWGQGTLVTVSSASTKGPSVFPLAPSSKSTSGGTAA |
| | hM2V4_PTGXLinker | LGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPEAA |
| | | GGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNKA |
| | | LPAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGN |
| | | VFSCSVMHEALHNHYTQKSLSLSPGKGGGGSGGGGSGGGGSQVQ |
| | | LVQSGAEVKKPGASVKVSCKASGYSFTTYTIHWVRQAPGQGLEWIG |
| | | YINPSSGYAAYNQKFQGRVTLTADKSTSTAYMELSSLRSEDTAVYYC |
| | | AREKFYGYDYWGQGTTVTVSSGGGGSGGGGSGGGGSGGGGASD |
| | | VVMTQSPDSLAVSLGERATINCKSSQSIVHRNGNTYLEWYQQKPGQ |
| | | PPKLLIYKVSNRFSGVPDRFSGSGSGTDFTLTISSLQAEDVAVYYCFQ |
| | | GSHVPYTFGGGTKLEIK |
| 60 | BEAT2b1 | DVVMTQSPDSLAVSLGERATINCKSSQSIVHRNGNTYLEWYQLKPG |
| | hM2V7_CLinker- | QPPKLLIYKVSNRFSGVPDRFSGSGSGTDFTLTISSLQAEDVAVYYCF |
| | (GGGGS)3- LC- | QGSHVPYTFGGGTKLEIKGGGGSGGGGSGGGGSQVQLVQSGAEV |
| | IgG1LALA(anti- | KKPGASVKVSCKASGYSFTTYTMHWVRQAPGQGLEWMGYINPSSG |
| | CD206) | YAAYNQKFQGRVTMTADKSTSTAYMELSSLRSEDTAVYYCAREKFY |
| | | GYDYWGQGTTVTVSSGGGGSGGGGSGGGGSDIQMTQSPSSLSAS |
| | | VGDRVTITCRASQGISRWLAWYQQKPEKAPKSLIYAASSLQSGVPSR |
| | | FSGSGSGTDFTLTISGLQPEDFATYYCQQYNSYPRTFGQGTKVEIKR |
| | | TVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGL |
| | | SSPVTKSFNRGEC |
| 61 | BEAT2b2 | DIQMTQSPSSLSASVGDRVTITCRASQGISRWLAWYQQKPEKAPKSL |
| | LC-IgG1LALA(anti- | IYAASSLQSGVPSRFSGSGSGTDFTLTISGLQPEDFATYYCQQYNSY |
| | CD206)-(GGGGS)3- | PRTFGQGTKVEIKRTVAAPSVFIFPPSDEQLKSGTASVVCLLNNFYPR |
| | hM2V7_Clinker | EAKVQWKVDNALQSGNSQESVTEQDSKDSTYSLSSTLTLSKADYEK |
| | | HKVYACEVTHQGLSSPVTKSFNRGECGGGGSGGGGSGGGGSDVV |
| | | MTQSPDSLAVSLGERATINCKSSQSIVHRNGNTYLEWYQLKPGQPP |
| | | KLLIYKVSNRFSGVPDRFSGSGSGTDFTLTISSLQAEDVAVYYCFQG |
| | | SHVPYTFGGGTKLEIKGGGGSGGGGSGGGGSQVQLVQSGAEVKKP |
| | | GASVKVSCKASGYSFTTYTMHWVRQAPGQGLEWMGYINPSSGYAA |
| | | YNQKFQGRVTMTADKSTSTAYMELSSLRSEDTAVYYCAREKFYGYD |
| | | YWGQGTTVTVSS |
| 62 | BEAT2b3 | DVVMTQSPDSLAVSLGERATINCKSSQSIVHRNGNTYLEWYQLKPG |
| | hM2V7_CLinker- | QPPKLLIYKVSNRFSGVPDRFSGSGSGTDFTLTISSLQAEDVAVYYCF |
| | (GGGGS)3- HC- | QGSHVPYTFGGGTKLEIKGGGGSGGGGSGGGGSQVQLVQSGAEV |
| | IgG1LALA(anti- | KKPGASVKVSCKASGYSFTTYTMHWVRQAPGQGLEWMGYINPSSG |
| | CD206) | YAAYNQKFQGRVTMTADKSTSTAYMELSSLRSEDTAVYYCAREKFY |
| | | GYDYWGQGTTVTVSSGGGGSGGGGSGGGGSEVQLVQSGAEVKKP |
| | | GESLRISCKGSGDSFTTYWIGWVRQMPGKGLEWMGIIYPGDSDTIYS |
| | | PSFQGQVTISADKSISTAYLQWSSLKASDTAMYYCTRGDRGVDYWG |
| | | QGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNV |
| | | NHKPSNTKVDKKVEPKSCDKTHTCPPCPAPEAAGGPSVFLFPPKPK |
| | | DTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKG |
| | | QPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALH |
| | | NHYTQKSLSLSPGK |
| 63 | BEAT2b4 | EVQLVQSGAEVKKPGESLRISCKGSGDSFTTYWIGWVRQMPGKGLE |
| | HC-IgG1LALA(anti- | WMGIIYPGDSDTIYSPSFQGQVTISADKSISTAYLQWSSLKASDTAMY |
| | CD206)-(GGGGS)3- | YCTRGDRGVDYWGQGTLVTVSSASTKGPSVFPLAPSSKSTSGGTAA |
| | hM2V7_Clinker | LGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPEAA |
| | | GGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNKA |
| | | LPAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGN |
| | | VFSCSVMHEALHNHYTQKSLSLSPGKGGGGSGGGGSGGGGSDVV |
| | | MTQSPDSLAVSLGERATINCKSSQSIVHRNGNTYLEWYQLKPGQPP |
| | | KLLIYKVSNRFSGVPDRFSGSGSGTDFTLTISSLQAEDVAVYYCFQG |
| | | SHVPYTFGGGTKLEIKGGGGSGGGGSGGGGSQVQLVQSGAEVKKP |
| | | GASVKVSCKASGYSFTTYTMHWVRQAPGQGLEWMGYINPSSGYAA |
| | | YNQKFQGRVTMTADKSTSTAYMELSSLRSEDTAVYYCAREKFYGYD |
| | | YWGQGTTVTVSS |

Linker and leader sequences

| | | |
|---|---|---|
| 64 | Linker (G3S)4 | GGGGSGGGGSGGGGS |
| 65 | Linker PTGX | GGGGSGGGGSGGGGSGGGGAS |
| 66 | Leader sequence-1 | MKHLWFFLLLVAAPRWVLS |
| 67 | Leader sequence-2 | MVLQTQVFISLLLWISGAYG |

CAR constructs

| | | |
|---|---|---|
| 68 | 7B1-CD28z | EVHLVESGGGLVQPGGSLRLSCAASGFTFSRYWMSWVRQAQGKGL |
| | | EWVASIKQAGSEKTYVDSVKGRFTISRDNAKNSLSLQMNSLRAEDTA |
| | | VYYCAREGAYYYDSASYYPYYYYYSMDVWGQGTTVTVSSGGGGSG |
| | | GGGSGGGGSEIVLTQSPGTLSLSPGERATLSCRASQSVSSSYLAWY |
| | | QQKPGQAPRLLIYGASSRATGIPDRFSGSGSGTDFTLTISRLEPEDFA |
| | | VYYCQQYGSSQYTFGQGTKLEIKGGGGSDYKDDDDKLSNSIMYFSH |
| | | FVPVFLPAKPTTTPAPRPPTPAPTIASQPLSLRPEACRPAAGGAVHTR |
| | | GLDFACDIYIWAPLAGTCGVLLLSLVITLYCNHRNRSKRSRLLHSDYM |
| | | NMTPRRPGPTRKHYQPYAPPRDFAAYRSLRVKFSRSADAPAYQQG |
| | | QNQLYNELNLGRREEYDVLDKRRGRDPEMGGKPRRKNPQEGLYNE |
| | | LQKDKMAEAYSEIGMKGERRRGKGHDGLYQGLSTATKDTYDALHM |
| | | QALPPR |
| 69 | 7B1-41BBz | EVHLVESGGGLVQPGGSLRLSCAASGFTFSRYWMSWVRQAQGKGL |
| | | EWVASIKQAGSEKTYVDSVKGRFTISRDNAKNSLSLQMNSLRAEDTA |
| | | VYYCAREGAYYYDSASYYPYYYYYSMDVWGQGTTVTVSSGGGGSG |
| | | GGGSGGGGSEIVLTQSPGTLSLSPGERATLSCRASQSVSSSYLAWY |
| | | QQKPGQAPRLLIYGASSRATGIPDRFSGSGSGTDFTLTISRLEPEDFA |
| | | VYYCQQYGSSQYTFGQGTKLEIKGGGGSDYKDDDDKLSNSIMYFSH |
| | | FVPVFLPAKPTTTPAPRPPTPAPTIASQPLSLRPEACRPAAGGAVHTR |
| | | GLDFACDIYIWAPLAGTCGVLLLSLVITLYCNHRNKRGRKKLLYIFKQP |
| | | FMRPVQTTQEEDGCSCRFPEEEEGGCELRVKFSRSADAPAYQQGQ |
| | | NQLYNELNLGRREEYDVLDKRRGRDPEMGGKPRRKNPQEGLYNEL |
| | | QKDKMAEAYSEIGMKGERRRGKGHDGLYQGLSTATKDTYDALHMQ |
| | | ALPPR |

FLAG binding polypeptides

| | | |
|---|---|---|
| 70 | Mouse anti-FLAG tag | QVQLQQSAGELARPGASVKMSCKASGYSFTTYTIHWVKQRPGQGL |
| | IgG1 VH | EWIGYINPSSGYAAYNQNFKDETTLTADKSSSTAYMQLSSLTSEDSA |
| | | VYYCAREKFYGYDYWGQGTTLTVSS |
| 71 | anti-FLAG tag hIgG1 | QVQLVQSGAEVKKPGASVKVSCKASGYSFTTYTIHWVRQAPGQGLE |
| | VH1 | WIGYINPSSGYAAYNQKFQGRVTLTADKSTSTAYMELSSLRSEDTAV |
| | | YYCAREKFYGYDYWGQGTTVTVSS |
| 72 | anti-FLAG tag hIgG1 | QVQLVQSGAEVKKPGASVKVSCKASGYSFTTYTMHWVRQAPGQGL |
| | VH2 | EWMGYINPSSGYAAYNQKFQGRVTMTADKSTSTAYMELSSLRSEDT |
| | | AVYYCAREKFYGYDYWGQGTTVTVSS |
| 73 | anti-FLAG tag hIgG1 | QVQLVQSGSELKKPGASVKVSCKASGYSFTTYTIHWVRQAPGQGLE |
| | VH3 | WIGYINPSSGYAAYNQGFTGRFVLSADKSVSTAYLQISSLKAEDTAVY |
| | | YCAREKFYGYDYWGQGTTVTVSS |
| 74 | anti-FLAG tag hIgG1 | QVQLVQSGSELKKPGASVKVSCKASGYSFTTYTMNWVRQAPGQGL |
| | VH4 | EWMGYINPSSGYAAYNQGFTGRFVFSADKSVSTAYLQISSLKAEDTA |
| | | VYYCAREKFYGYDYWGQGTTVTVSS |
| 75 | Mouse anti-FLAG tag | DVLMTQLPLSLPVSLGDQASISCRSSQSIVHRNGNTYLEWYLLKPGQ |
| | IgG1 VL | SPKLLIYKVSNRFSGVPDRFSGSGSGTDFTLKISRVEAEDLGVYYCF |
| | | QGSHVPYTFGGGTKLEIK |
| 76 | anti-FLAG tag IgG1 | DVVMTQSPLSLPVTLGQPASISCRSSQSIVHRNGNTYLEWFQLRPG |
| | VL1 | QSPRLLIYKVSNRFSGVPDRFSGSGSGTDFTLKISRVEAEDVGVYYC |
| | | FQGSHVPYTFGGGTKLEIK |
| 77 | anti-FLAG tag IgG1 | DVVMTQSPLSLPVTLGQPASISCRSSQSIVHRNGNTYLEWFQQRPG |
| | VL2 | QSPRLLIYKVSNRFSGVPDRFSGSGSGTDFTLKISRVEAEDVGVYYC |
| | | FQGSHVPYTFGGGTKLEIK |
| 78 | anti-FLAG tag IgG1 | DVVMTQSPDSLAVSLGERATINCKSSQSIVHRNGNTYLEWYQLKPG |
| | VL3 | QPPKLLIYKVSNRFSGVPDRFSGSGSGTDFTLTISSLQAEDVAVYYCF |
| | | QGSHVPYTFGGGTKLEIK |
| 79 | anti-FLAG tag IgG1 | DVVMTQSPDSLAVSLGERATINCKSSQSIVHRNGNTYLEWYQQKPG |
| | VL4 | QPPKLLIYKVSNRFSGVPDRFSGSGSGTDFTLTISSLQAEDVAVYYCF |
| | | QGSHVPYTFGGGTKLEIK |
| 80 | FLAG tag | DYKD |
| 81 | CDR H1 parental, | GYSFTTYT |
| | VH1, VH2, VH3 and | |
| | VH4 (IMGT) | |
| 82 | CDR H2 parental, | INPSSGYA |
| | VH1, VH2, VH3 and | |
| | VH4 (IMGT) | |
| 83 | CDR H3 parental, | AREKFYGYDY |
| | VH1, VH2, VH3 and | |
| | VH4 (IMGT) | |
| 84 | FR H1 VH1 and VH2 | QVQLVQSGAEVKKPGASVKVSCKAS |
| | (IMGT and Chothia) | |
| 85 | FR H2 VH1 (IMGT) | IHWVRQAPGQGLEWIGY |
| 86 | FR H2 VH2 (IMGT) | MHWVRQAPGQGLEWMGY |
| 87 | FR H3 VH1 and VH3 | AYNQKFQGRVTLTADKSTSTAYMELSSLRSEDTAVYYC |
| | (IMGT) | |
| 88 | FR H3 VH2 (IMGT) | AYNQKFQGRVTMTADKSTSTAYMELSSLRSEDTAVYYC |
| 89 | FR H4 VH1, VH2, | WGQGTTVTVSS |
| | VH3 and VH4 (IMGT, | |
| | Kabat and Chothia) | |
| 90 | CDR L1 parental, | QSIVHRNGNTY |
| | VL1, VL2, VL3 and | |
| | VL4 (IMGT) | |
| 91 | CDR L2 parental, | KVS |
| | VL1, VL2, VL3 and | |
| | VL4 (IMGT) | |
| 92 | CDR L3 parental, | FQGSHVPYT |
| | VL1, VL2, VL3 and | |
| | VL4 (IMGT, Kabat | |
| | and Chothia) | |
| 93 | FR L1 VL3 and VL4 | DVVMTQSPDSLAVSLGERATINCKSS |
| | (IMGT) | |
| 94 | FR L2 VL4 (IMGT) | LEWYQQKPGQPPKLLIY |
| 95 | FR L2 VL3 (IMGT) | LEWYQLKPGQPPKLLIY |
| 96 | FR L3 VL3 and VL4 | NRFSGVPDRFSGSGSGTDFTLTISSLQAEDVAVYYC |
| | (IMGT) | |
| 97 | FR L4 parental, VL1, | FGGGTKLEIK |
| | VL2, VL3 and VL4 | |
| | (IMGT, Kabat and | |
| | Chothia) | |
| 98 | FLAG tag deca- | GDYKDDDDKG |
| | peptide | |
| 99 | FLAG tag | DYKDDDDK |
| | octapeptide | |
| 100 | FLAG tag | MDYKDDDDK |
| | nonapeptide | |
| 101 | FLAG tag | DFKDDDK |
| | heptapeptide | |
| 102 | FLAG tag alternative | DYKAFDNL |
| | octapeptide | |
| 103 | FLAG tag alternative | DYKDHDG |
| | heptapeptide | |
| 104 | FLAG tag alternative | MDFKDDDDK |
| 105 | FLAG tag alternative | MDYKAFDNL |
| 106 | FLAG tag alternative | DYKDHDI |
| 107 | FLAG tag alternative | DYKDH |
| 108 | FLAG tag alternative | DYKDD |
| 109 | FLAG tag alternative | DYKDHD |
| 110 | FLAG tag alternative | DYKDDD |
| 111 | CDR H1 parental, | TYTIH |
| | VH1 and VH3 | |
| | (Kabat) | |
| 112 | CDR H1 VH2 (Kabat) | TYTMH |
| 113 | CDR H2 VH1 and | YINPSSGYAAYNQKFQG |
| | VH2 (Kabat) | |
| 114 | CDR H3 parental, | EKFYGYDY |
| | VH1, VH2, VH3 and | |
| | VH4 (Kabat and | |
| | Chothia) | |
| 115 | FR H1 VH1 and VH2 | QVQLVQSGAEVKKPGASVKVSCKASGYSF |
| | (Kabat) | |
| 116 | FR H2 VH1 and VH3 | WVRQAPGQGLEWIG |
| | (Kabat) | |
| 117 | FR H2 VH2 and VH4 | WVRQAPGQGLEWMG |
| | (Kabat) | |
| 118 | FR H3 VH1 (Kabat) | RVTLTADKSTSTAYMELSSLRSEDTAVYYCAR |
| 119 | FR H3 VH2 (Kabat) | RVTMTADKSTSTAYMELSSLRSEDTAVYYCAR |
| 120 | CDR L1 VL3 and | KSSQSIVHRNGNTYLE |
| | VL4 (Kabat and | |
| | Chothia) | |
| 121 | CDR L2 parental, | KVSNRFS |
| | VL1, VL2, VL3 and | |
| | VL4 (Kabat and | |
| | Chothia) | |
| 122 | FR L1 VL3 and VL4 | DVVMTQSPDSLAVSLGERATINC |
| | (Kabat and Chothia) | |
| 123 | FR L2 VL4 (Kabat | WYQQKPGQPPKLLIY |
| | and Chothia) | |
| 124 | FR L2 VL3 (Kabat | WYQLKPGQPPKLLIY |
| | and Chothia) | |
| 125 | FR L3 VL3 and VL4 | GVPDRFSGSGSGTDFTLTISSLQAEDVAVYYC |
| | (Kabat and Chothia) | |
| 126 | CDR H1 parental, | GYSFTTY |
| | VH1, VH2, VH3 and | |
| | VH4 (Chothia) | |
| 127 | CDR H2 parental, | NPSSGY |
| | VH1, VH2, VH3 and | |
| | VH4 (Chothia) | |
| 128 | FR H2 VH1 and VH3 | TIHWVRQAPGQGLEWIGYI |
| | (Chothia) | |
| 129 | FR H2 VH2 (Chothia) | TMHWVRQAPGQGLEWMGYI |
| 130 | FR H3 VH1 (Chothia) | AAYNQKFQGRVTLTADKSTSTAYMELSSLRSEDTAVYYCAR |
| 131 | FR H3 VH2 (Chothia) | AAYNQKFQGRVTMTADKSTSTAYMELSSLRSEDTAVYYCAR |
| 132 | CDR H2 parental | YINPSSGYAAYNQNFKD |
| | (Kabat) | |
| 133 | CDR L1 parental VL1 | RSSQSIVHRNGNTYLE |
| | and VL2 (Kabat and | |
| | Chothia) | |
| 134 | FR H1 parental | QVQLQQSAGELARPGASVKMSCKAS |
| | (IMGT) | |
| 135 | FR H2 parental | IHWVKQRPGQGLEWIGY |
| | (IMGT) | |
| 136 | FR H3 parental | AYNQNFKDETTLTADKSSSTAYMQLSSLTSEDSAVYYC |
| | (IMGT) | |
| 137 | FR H4 parental | WGQGTTLTVSS |
| | (IMGT, Kabat and | |
| | Chothia) | |
| 138 | FR L1 parental and | DVLMTQLPLSLPVSLGDQASISCRSS |
| | VL1 (IMGT) | |
| 139 | FR L2 parental and | LEWYLLKPGQSPKLLIY |
| | VL1 (IMGT) | |
| 140 | FR L3 parental and | NRFSGVPDRFSGSGSGTDFTLKISRVEAEDLGVYYC |
| | VL1 (IMGT) | |
| 141 | FR H1 parental | QVQLQQSAGELARPGASVKMSCKASGYSFT |
| | (Kabat) | |
| 142 | FR H2 parental | WVKQRPGQGLEWIG |
| | (Kabat) | |
| 143 | FR H3 parental | ETTLTADKSSSTAYMQLSSLTSEDSAVYYCAR |
| | (Kabat) | |
| 144 | FR L1 parental and | DVLMTQLPLSLPVSLGDQASISC |
| | VL1 (Kabat and | |
| | Chothia) | |
| 145 | FR L2 parental and | WYLLKPGQSPKLLIY |
| | VL1 (Kabat and | |
| | Chothia) | |
| 146 | FR L3 parental and | GVPDRFSGSGSGTDFTLKISRVEAEDLGVYYCF |
| | VL1 (Kabat and | |
| | Chothia) | |
| 147 | FR H2 parental | TIHWVKQRPGQGLEWIGYI |
| | (Chothia) | |
| 148 | FR H3 parental | AAYNQNFKDETTLTADKSSSTAYMQLSSLTSEDSAVYYCAR |
| | (Chothia) | |
| 149 | FR H1 VH3 and VH4 | QVQLVQSGSELKKPGASVKVSCKAS |
| | (IMGT) | |
| 150 | FR H2 VH4 (IMGT) | MNWVRQAPGQGLEWMGY |
| 151 | FR H3 VH3 (IMGT) | AYNQGFTGRFVLSADKSVSTAYLQISSLKAEDTAVYYC |
| 152 | FR H3 VH4 (IMGT) | AYNQGFTGRFVFSADKSVSTAYLQISSLKAEDTAVYYC |
| 153 | FR L1 VL2 (IMGT) | DVVMTQSPLSLPVTLGQPASISCRSS |
| 154 | FR L2 VL2 (IMGT) | LEWFQQRPGQSPRLLIY |
| 155 | FR L3 VL2 (IMGT) | NRFSGVPDRFSGSGSGTDFTLKISRVEAEDVGVYYC |
| 156 | CDR H1 VH4 (Kabat) | TYTMN |
| 157 | CDR H2 VH3 and | YINPSSGYAAYNQGFTG |
| | VH4 (Kabat) | |
| 158 | FR H1 VH3 and VH4 | QVQLVQSGSELKKPGASVKVSCKASGYSFT |
| | (Kabat) | |
| 159 | FR H3 VH3 (Kabat) | RFVLSADKSVSTAYLQISSLKAEDTAVYYCAR |
| 160 | FR H3 VH4 (Kabat) | RFVFSADKSVSTAYLQISSLKAEDTAVYYCAR |
| 161 | FR L1 VL2 (Kabat) | DVVMTQSPLSLPVTLGQPASISC |
| 162 | FR L2 VL2 (Kabat) | WFQQRPGQSPRLLIY |
| 163 | FR L3 VL2 (Kabat) | GVPDRFSGSGSGTDFTLKISRVEAEDVGVYYC |
| 164 | FR H1 VH3 and VH4 | QVQLVQSGSELKKPGASVKVSCKAS |
| | Chothia) | |
| 165 | FR H2 VH4 (Chothia) | TMNWVRQAPGQGLEWMGYI |
| 166 | FR H3 VH3 (Chothia) | AAYNQGFTGRFVLSADKSVSTAYLQISSLKAEDTAVYYCAR |
| 167 | FR H3 VH4 (Chothia) | AAYNQGFTGRFVFSADKSVSTAYLQISSLKAEDTAVYYCAR |
| 168 | FR L1 VL2 (Chothia) | DVVMTQSPLSLPVTLGQPASISC |
| 169 | FR L2 VL2 (Chothia) | WFQQRPGQSPRLLIY |
| 170 | FR L3 VL2 (Chothia) | GVPDRFSGSGSGTDFTLKISRVEAEDVGVYYC |
| 171 | BEAT 3 and 5 | MKHLWFFLLLVAAPRWVLSQVQLVQSGAEVKKPGASVKVSCKASGY |
| | HC-IgG2(anti-CD40)- | TFTGYYMHWVRQAPGQGLEWMGWINPDSGGTNYAQKFQGRVTMT |
| | KIH1a | RDTSISTAYMELNRLRSDDTAVYYCARDQPLGYCTNGVCSYFDYWG |
| | | QGTLVTVSSASTKGPSVFPLAPCSRSTSESTAALGCLVKDYFPEPVT |
| | | VSWNSGALTSGVHTFPAVLQSSGLYSLSSVVTVPSSNFGTQTYTCN |
| | | VDHKPSNTKVDKTVERKCCVECPPCPAPPVAGPSVFLFPPKPKDTL |
| | | MISRTPEVTCVVVDVSHEDPEVQFNWYVDGVEVHNAKTKPREEQFN |
| | | STFRVVSVLTVVHQDWLNGKEYKCKVSNKGLPAPIEKTISKTKGQPR |
| | | EPQVYTLPPCREEMTKNQVSLWCLVKGFYPSDIAVEWESNGQPENN |
| | | YKTTPPMLDSDGSFFLYSKLTVDKSRWQQGNVFSCSVMHEALHNHY |
| | | TQKSLSLSPGK |
| 172 | BEAT 3, 4, 5, 6 | MVLQTQVFISLLLWISGAYGDIQMTQSPSSVSASVGDRVTITCRASQ |
| | LC-IgG2(anti-CD40) | GIYSWLAWYQQKPGKAPNLLIYTASTLQSGVPSRFSGSGSGTDFTLT |
| | | ISSLQPEDFATYYCQQANIFPLTFGGGTKVEIKRTVAAPSVFIFPPSDE |
| | | QLKSGTASVVCLLNNFYPREAKVQWKVDNALQSGNSQESVTEQDS |
| | | KDSTYSLSSTLTLSKADYEKHKVYACEVTHQGLSSPVTKSFNRGEC |
| 173 | BEAT 3 | MKHLWFFLLLVAAPRWVLSDVVMTQSPDSLAVSLGERATINCKSSQ |
| | HC- | SIVHRNGNTYLEWYQQKPGQPPKLLIYKVSNRFSGVPDRFSGSGSG |
| | hM2V4_CurrusLinker- | TDFTLTISSLQAEDVAVYYCFQGSHVPYTFGGGTKLEIKGGGGSGGG |
| | (GGGGS)3-IgG2Fc- | GSGGGGSQVQLVQSGAEVKKPGASVKVSCKASGYSFTTYTIHWVR |
| | KIH1b | QAPGQGLEWIGYINPSSGYAAYNQKFQGRVTLTADKSTSTAYMELS |
| | | SLRSEDTAVYYCAREKFYGYDYWGQGTTVTVSSGGGGSGGGGSG |
| | | GGGSERKCCVECPPCPAPPVAGPSVFLFPPKPKDTLMISRTPEVTCV |
| | | VVDVSHEDPEVQFNWYVDGVEVHNAKTKPREEQFNSTFRVVSVLTV |
| | | VHQDWLNGKEYKCKVSNKGLPAPIEKTISKTKGQPREPQVCTLPPSR |
| | | EEMTKNQVSLSCAVKGFYPSDIAVEWESNGQPENNYKTTPPMLDSD |
| | | GSFFLVSKLTVDKSRWQQGNVFSCSVMHEALHNHYTQKSLSLSPGK |
| 174 | BEAT 4 and 6 | MKHLWFFLLLVAAPRWVLSQVQLVQSGAEVKKPGASVKVSCKASGY |
| | HC-IgG2(anti-CD40)- | TFTGYYMHWVRQAPGQGLEWMGWINPDSGGTNYAQKFQGRVTMT |
| | KIH2a | RDTSISTAYMELNRLRSDDTAVYYCARDQPLGYCTNGVCSYFDYWG |
| | | QGTLVTVSSASTKGPSVFPLAPCSRSTSESTAALGCLVKDYFPEPVT |
| | | VSWNSGALTSGVHTFPAVLQSSGLYSLSSVVTVPSSNFGTQTYTCN |
| | | VDHKPSNTKVDKTVERKCCVECPPCPAPPVAGPSVFLFPPKPKDTL |
| | | MISRTPEVTCVVVDVSHEDPEVQFNWYVDGVEVHNAKTKPREEQFN |
| | | STFRVVSVLTVVHQDWLNGKEYKCKVSNKGLPAPIEKTISKTKGQPR |
| | | EPQVYTLPPSREEMTKNQVSLYCLVKGFYPSDIAVEWESNGQPENN |
| | | YKTTPPMLDSDGSFFLYSKLTVDKSRWQQGNVFSCSVMHEALHNHY |
| | | TQKSLSLSPGK |
| 175 | BEAT 4 | MKHLWFFLLLVAAPRWVLSDVVMTQSPDSLAVSLGERATINCKSSQ |
| | HC- | SIVHRNGNTYLEWYQQKPGQPPKLLIYKVSNRFSGVPDRFSGSGSG |
| | hM2V4_CurrusLinker- | TDFTLTISSLQAEDVAVYYCFQGSHVPYTFGGGTKLEIKGGGGSGGG |
| | (GGGGS)3-lgG2Fc- | GSGGGGSQVQLVQSGAEVKKPGASVKVSCKASGYSFTTYTIHWVR |
| | KIH2b | QAPGQGLEWIGYINPSSGYAAYNQKFQGRVTLTADKSTSTAYMELS |
| | | SLRSEDTAVYYCAREKFYGYDYWGQGTTVTVSSGGGGSGGGGSG |
| | | GGGSERKCCVECPPCPAPPVAGPSVFLFPPKPKDTLMISRTPEVTCV |
| | | VVDVSHEDPEVQFNWYVDGVEVHNAKTKPREEQFNSTFRVVSVLTV |
| | | VHQDWLNGKEYKCKVSNKGLPAPIEKTISKTKGQPREPQVYTLPPSR |
| | | EEMTKNQVSLTCLVKGFYPSDIAVEWESNGQPENNYKTTPPMLDSD |
| | | GSFFLTSKLTVDKSRWQQGNVFSCSVMHEALHNHYTQKSLSLSPGK |
| 176 | BEAT 5 | MKHLWFFLLLVAAPRWVLSQVQLVQSGAEVKKPGASVKVSCKASGY |
| | HC-IgG2(anti-CD40)- | TFTGYYMHWVRQAPGQGLEWMGWINPDSGGTNYAQKFQGRVTMT |
| | (GGGGS)3- | RDTSISTAYMELNRLRSDDTAVYYCARDQPLGYCTNGVCSYFDYWG |
| | hM2V4_CurrusLinker- | QGTLVTVSSASTKGPSVFPLAPCSRSTSESTAALGCLVKDYFPEPVT |
| | KIH1b | VSWNSGALTSGVHTFPAVLQSSGLYSLSSVVTVPSSNFGTQTYTCN |
| | | VDHKPSNTKVDKTVERKCCVECPPCPAPPVAGPSVFLFPPKPKDTL |
| | | MISRTPEVTCVVVDVSHEDPEVQFNWYVDGVEVHNAKTKPREEQFN |
| | | STFRVVSVLTVVHQDWLNGKEYKCKVSNKGLPAPIEKTISKTKGQPR |
| | | EPQVCTLPPSREEMTKNQVSLSCAVKGFYPSDIAVEWESNGQPENN |
| | | YKTTPPMLDSDGSFFLVSKLTVDKSRWQQGNVFSCSVMHEALHNHY |
| | | TQKSLSLSPGKGGGGSGGGGSGGGGSDVVMTQSPDSLAVSLGER |
| | | ATINCKSSQSIVHRNGNTYLEWYQQKPGQPPKLLIYKVSNRFSGVPD |
| | | RFSGSGSGTDFTLTISSLQAEDVAVYYCFQGSHVPYTFGGGTKLEIK |
| | | GGGGSGGGGSGGGGSQVQLVQSGAEVKKPGASVKVSCKASGYSF |
| | | TTYTIHWVRQAPGQGLEWIGYINPSSGYAAYNQKFQGRVTLTADKST |
| | | STAYMELSSLRSEDTAVYYCAREKFYGYDYWGQGTTVTVSS |
| 177 | BEAT 6 | MKHLWFFLLLVAAPRWVLSQVQLVQSGAEVKKPGASVKVSCKASGY |
| | HC-IgG2(anti-CD40)- | TFTGYYMHWVRQAPGQGLEWMGWINPDSGGTNYAQKFQGRVTMT |
| | (GGGGS)3- | RDTSISTAYMELNRLRSDDTAVYYCARDQPLGYCTNGVCSYFDYWG |
| | hM2V4_CurrusLinker- | QGTLVTVSSASTKGPSVFPLAPCSRSTSESTAALGCLVKDYFPEPVT |
| | KIH2b | VSWNSGALTSGVHTFPAVLQSSGLYSLSSVVTVPSSNFGTQTYTCN |
| | | VDHKPSNTKVDKTVERKCCVECPPCPAPPVAGPSVFLFPPKPKDTL |
| | | MISRTPEVTCVVVDVSHEDPEVQFNWYVDGVEVHNAKTKPREEQFN |
| | | STFRVVSVLTVVHQDWLNGKEYKCKVSNKGLPAPIEKTISKTKGQPR |
| | | EPQVYTLPPSREEMTKNQVSLTCLVKGFYPSDIAVEWESNGQPENN |
| | | YKTTPPMLDSDGSFFLTSKLTVDKSRWQQGNVFSCSVMHEALHNHY |
| | | TQKSLSLSPGKGGGGSGGGGSGGGGSDVVMTQSPDSLAVSLGER |
| | | ATINCKSSQSIVHRNGNTYLEWYQQKPGQPPKLLIYKVSNRFSGVPD |
| | | RFSGSGSGTDFTLTISSLQAEDVAVYYCFQGSHVPYTFGGGTKLEIK |
| | | GGGGSGGGGSGGGGSQVQLVQSGAEVKKPGASVKVSCKASGYSF |
| | | TTYTIHWVRQAPGQGLEWIGYINPSSGYAAYNQKFQGRVTLTADKST |
| | | STAYMELSSLRSEDTAVYYCAREKFYGYDYWGQGTTVTVSS |
| 178 | DNA BEAT1a4 | CAAGTGCAACTAGTGCAAAGTGGTGCAGAAGTCAAGAAGCCCGG |
| | HC | CGCTTCCGTGAAGGTATCATGCAAGGCCTCTGGATACACCTTCAC |
| | | CGGCTACTACATGCATTGGGTCAGACAGGCCCCCGGACAAGGTC |
| | | TGGAATGGATGGGCTGGATCAACCCTGACTCCGGCGGAACCAAC |
| | | TACGCCCAGAAGTTTCAGGGACGCGTGACCATGACCAGAGACAC |
| | | AAGCATCTCCACCGCTTATATGGAGCTGAACCGGCTGAGATCCGA |
| | | CGACACCGCCGTGTACTACTGTGCTAGAGACCAGCCTCTGGGCT |
| | | ACTGCACCAATGGCGTGTGCTCCTACTTCGACTACTGGGGCCAG |
| | | GGAACCCTGGTGACAGTGTCCTCTGCCAGCACCAAAGGCCCTTC |
| | | TGTGTTCCCCCTGGCCCCTTGCTCTCGGAGCACCTCCGAGTCTAC |
| | | CGCTGCTCTGGGCTGCCTGGTGAAGGATTACTTTCCCGAGCCTG |
| | | TGACCGTCTCTTGGAACTCCGGCGCCCTCACCTCCGGCGTGCAC |
| | | ACCTTTCCTGCTGTGCTGCAGTCCAGCGGCCTGTACTCTCTGTCC |
| | | TCTGTGGTGACCGTGCCTTCTTCCAACTTCGGCACACAGACCTAC |
| | | ACCTGCAACGTGGACCACAAGCCTAGCAATACCAAGGTAGATAAG |
| | | ACGGTGGAACGGAAGTGCTGCGTGGAGTGCCCTCCGTGTCCTGC |
| | | CCCTCCTGTGGCAGGCCCATCTGTGTTTCTGTTCCCTCCAAAGCC |
| | | CAAGGACACCCTGATGATCTCCAGAACCCCTGAGGTGACCTGTGT |
| | | GGTGGTCGATGTGTCTCACGAGGACCCTGAAGTGCAGTTCAACT |
| | | GGTACGTGGACGGAGTGGAAGTGCATAACGCCAAGACCAAACCT |
| | | CGGGAAGAGCAGTTCAACAGCACCTTCAGAGTGGTGTCCGTGCT |
| | | GACCGTGGTACACCAGGACTGGCTGAACGGCAAAGAGTACAAGT |
| | | GCAAGGTGTCTAACAAGGGCCTGCCTGCTCCAATCGAGAAGACG |
| | | ATTTCCAAAACAAAGGGCCAGCCCAGAGAACCTCAGGTGTACACA |
| | | CTGCCTCCATCTAGGGAGGAAATGACCAAGAACCAGGTGTCCCT |
| | | GACCTGTCTGGTCAAGGGCTTCTACCCCTCCGACATCGCCGTGG |
| | | AATGGGAGAGCAATGGACAGCCTGAGAACAACTACAAAACCACAC |
| | | CTCCTATGCTGGACTCTGACGGCTCATTCTTCCTGTACTCCAAGC |
| | | TGACCGTGGATAAGTCCAGATGGCAGCAGGGCAATGTGTTCAGC |
| | | TGTTCTGTGATGCACGAGGCCCTGCACAACCACTACACCCAGAAG |
| | | TCCCTGTCTCTCAGCCCTGGGAAGGGCGGAGGAGGCTCCGGAG |
| | | GCGGCGGCTCTGGAGGTGGAGGCAGCGATGTCGTGATGACCCA |
| | | ATCTCCTGATAGCCTGGCTGTGTCCTTGGGCGAGAGAGCCACCA |
| | | TCAACTGCAAGTCTTCTCAGTCTATCGTGCACCGGAACGGCAACA |
| | | CCTACCTCGAGTGGTACCAGCAGAAACCAGGCCAACCTCCTAAG |
| | | CTGCTGATCTACAAGGTGTCCAACAGATTCTCTGGCGTCCCCGAC |
| | | CGGTTCTCCGGCTCCGGCTCCGGCACCGACTTCACCCTGACCAT |
| | | CTCCTCCCTGCAGGCTGAGGACGTGGCCGTGTATTATTGCTTCCA |
| | | GGGCTCCCACGTGCCCTACACCTTTGGCGGCGGCACCAAGCTGG |
| | | AGATCAAGGGTGGCGGCGGCTCTGGCGGCGGCGGCTCCGGAGG |
| | | TGGTGGCTCCCAAGTGCAGCTGGTGCAGTCCGGCGCCGAGGTG |
| | | AAGAAACCTGGCGCTTCTGTGAAAGTGTCCTGCAAGGCCTCTGG |
| | | CTACTCCTTCACCACCTATACAATCCACTGGGTGCGGCAGGCCCC |
| | | TGGCCAAGGCCTGGAATGGATCGGCTACATCAACCCCTCCAGTG |
| | | GCTACGCCGCTTACAACCAGAAGTTCCAGGGCAGAGTGACCCTG |
| | | ACCGCCGACAAGTCCACCTCTACAGCCTACATGGAACTGTCCTCT |
| | | CTGCGGTCTGAGGATACCGCTGTGTACTACTGCGCTAGAGAGAA |
| | | GTTCTACGGCTACGACTACTGGGGCCAGGGCACCACCGTGACAG |
| | | TCTCCTCT |
| 179 | DNA BEAT1a4 | GATATCCAAATGACTCAAAGTCCAAGTAGTGTGTCCGCTTCCGTG |
| | LC | GGCGACCGCGTGACCATCACCTGTAGAGCCTCGCAGGGCATCTA |
| | | TTCTTGGCTGGCTTGGTACCAGCAGAAGCCTGGCAAGGCTCCTAA |
| | | TCTGCTGATCTACACCGCCTCTACCCTCCAGTCCGGCGTGCCTTC |
| | | CCGGTTCTCCGGCTCTGGAAGCGGCACCGACTTCACCCTGACTA |
| | | TCTCCTCTCTGCAGCCCGAGGACTTTGCTACCTACTACTGCCAGC |
| | | AGGCCAACATCTTCCCTCTGACCTTCGGCGGAGGAACCAAAGTG |
| | | GAAATCAAGCGGACCGTGGCCGCCCCTAGCGTGTTCATCTTTCCA |
| | | CCTTCTGATGAACAACTGAAGTCTGGCACCGCTTCTGTGGTGTGC |
| | | CTGCTGAACAACTTCTACCCTAGAGAGGCCAAAGTGCAGTGGAAG |
| | | GTGGACAACGCCCTGCAGTCTGGCAACTCCCAAGAGTCCGTCAC |
| | | CGAGCAGGATTCTAAGGACTCCACCTACTCCCTGTCTAGCACACT |
| | | GACACTGTCCAAGGCCGACTACGAGAAGCACAAGGTGTACGCCT |
| | | GCGAAGTGACCCACCAGGGCCTGAGCTCCCCCGTCACAAAGTCC |
| | | TTCAACAGAGGCGAGTGC |
| 180 | DNA BEAT2B3 | GATGTGGTGATGACTCAAAGTCCAGATAGTCTGGCCGTGAGCCT |
| | HC | GGGCGAGAGAGCCACCATCAACTGCAAGTCTAGCCAGAGCATCG |
| | | TGCACAGAAACGGCAACACCTACCTGGAATGGTACCAGCTGAAG |
| | | CCCGGTCAGCCTCCTAAGCTGCTGATCTACAAGGTGTCCAACAGA |
| | | TTCTCCGGAGTGCCTGATCGGTTCTCTGGCTCCGGCTCTGGCAC |
| | | CGACTTCACCCTGACCATCTCCTCTCTGCAGGCTGAGGATGTGGC |
| | | CGTGTACTATTGCTTCCAGGGCTCCCATGTGCCCTACACCTTCGG |
| | | CGGCGGCACAAAGCTGGAGATCAAGGGCGGAGGCGGCAGCGGC |
| | | GGAGGCGGCAGTGGCGGTGGCGGCTCCCAGGTGCAGCTGGTGC |
| | | AGTCTGGCGCCGAGGTGAAGAAGCCCGGAGCTTCTGTGAAAGTG |
| | | TCCTGTAAGGCCTCTGGCTACAGCTTCACCACATATACCATGCAC |
| | | TGGGTCAGACAGGCTCCTGGACAAGGACTGGAGTGGATGGGCTA |
| | | CATCAACCCTTCCAGCGGATACGCCGCCTACAACCAGAAGTTTCA |
| | | GGGCAGAGTGACCATGACCGCTGACAAGTCCACCTCTACCGCTT |
| | | ACATGGAACTGAGCTCCCTGAGATCCGAGGACACTGCAGTGTACT |
| | | ACTGTGCCAGAGAGAAGTTCTACGGCTACGACTACTGGGGCCAG |
| | | GGAACAACCGTTACCGTGTCCTCCGGTGGCGGCGGCTCTGGAGG |
| | | CGGGGGCTCCGGCGGCGGAGGCTCTGAAGTGCAGCTGGTGCAG |
| | | TCTGGCGCAGAAGTGAAGAAACCTGGCGAGAGCCTGCGGATCTC |
| | | CTGCAAGGGATCCGGCGATTCTTTTACCACCTACTGGATCGGCTG |
| | | GGTGCGGCAGATGCCTGGCAAGGGCCTGGAGTGGATGGGCATC |
| | | ATCTACCCTGGCGACTCTGATACCATCTATTCTCCTTCCTTCCAGG |
| | | GCCAAGTCACCATCTCTGCCGACAAGTCCATCAGCACCGCCTACC |
| | | TGCAGTGGTCCTCCCTGAAGGCCTCCGACACCGCCATGTACTACT |
| | | GCACCCGGGGCGACAGAGGCGTCGACTATTGGGGCCAGGGCAC |
| | | CCTCGTGACCGTCTCCAGCGCCTCTACAAAAGGCCCTTCCGTCTT |
| | | CCCTCTGGCTCCTTCTTCTAAATCTACCAGCGGCGGCACCGCTGC |
| | | TCTGGGCTGTCTGGTGAAGGATTACTTCCCCGAGCCCGTGACAG |
| | | TGTCATGGAACTCTGGCGCCCTGACCTCTGGCGTGCATACATTCC |
| | | CTGCTGTGCTGCAGTCCTCTGGCCTGTACTCCCTGAGTTCTGTGG |
| | | TGACCGTGCCTTCGTCCTCCCTCGGCACCCAGACATACATCTGCA |
| | | ACGTGAACCACAAGCCCTCCAACACCAAAGTCGACAAGAAGGTG |
| | | GAACCTAAGTCTTGCGACAAAACCCACACCTGTCCTCCTTGCCCC |
| | | GCCCCGGAGGCCGCTGGCGGACCTAGCGTGTTTCTGTTCCCTCC |
| | | TAAGCCCAAGGACACACTGATGATCTCTCGGACCCCTGAGGTTAC |
| | | CTGCGTGGTCGTGGACGTTTCCCACGAAGATCCTGAGGTGAAGT |
| | | TCAACTGGTACGTGGATGGCGTGGAAGTGCACAACGCCAAGACC |
| | | AAGCCTCGGGAGGAACAGTACAATTCCACTTACAGAGTGGTCTCT |
| | | GTGCTGACCGTCCTGCACCAGGACTGGCTGAACGGCAAAGAGTA |
| | | CAAGTGCAAGGTGTCAAACAAGGCTCTCCCAGCCCCTATCGAGAA |
| | | GACCATTTCCAAGGCTAAGGGCCAGCCTCGCGAGCCCCAAGTGT |
| | | ACACCCTGCCACCTTCTAGGGAAGAGATGACTAAGAATCAAGTGT |
| | | CCCTTACATGCCTGGTGAAAGGGTTCTATCCTTCTGACATCGCCG |
| | | TGGAGTGGGAGTCTAATGGCCAGCCAGAGAACAACTACAAGACC |
| | | ACCCCTCCTGTGCTGGACTCCGACGGCTCTTTCTTCCTGTACTCC |
| | | AAGCTGACAGTGGACAAGAGCAGATGGCAGCAGGGCAACGTGTT |
| | | TTCCTGCTCCGTGATGCACGAAGCTCTGCACAACCACTACACCCA |
| | | GAAGTCCCTGTCCCTGTCTCCGGGCAAG |
| 181 | DNA BEAT2B3 | GATATCCAAATGACTCAAAGTCCAAGTAGTCTGTCTGCTTCTGTG |
| | LC | GGCGACAGAGTGACCATCACCTGCCGCGCTAGCCAGGGCATCTC |
| | | TAGATGGCTGGCCTGGTACCAGCAGAAGCCCGAGAAAGCCCCTA |
| | | AGTCCCTGATCTACGCTGCCTCTTCTCTGCAGTCTGGCGTGCCTT |
| | | CTCGGTTCTCCGGCAGCGGCTCCGGCACCGATTTTACACTGACA |
| | | ATCTCCGGTCTCCAGCCTGAGGACTTCGCCACCTACTACTGCCAA |
| | | CAGTACAATTCCTACCCTAGAACCTTTGGCCAGGGCACCAAGGTG |
| | | GAAATCAAGCGGACCGTGGCTGCTCCCAGCGTGTTCATCTTCCCT |
| | | CCTTCCGACGAACAGCTGAAGTCCGGAACCGCCTCTGTGGTGTG |
| | | CCTGCTGAACAACTTCTATCCTCGGGAAGCTAAAGTGCAGTGGAA |
| | | GGTCGATAACGCCCTGCAGTCCGGCAACTCTCAAGAGTCCGTGA |
| | | CCGAGCAGGACTCCAAGGACTCTACCTACTCCCTGAGCTCCACC |
| | | CTGACCCTGAGCAAGGCCGACTACGAGAAGCACAAGGTGTACGC |
| | | CTGCGAGGTGACCCACCAGGGACTGTCCTCCCCAGTCACAAAGT |
| | | CCTTCAACAGAGGCGAGTGT |
| 182 | DNA BEAT1a4 | ATGAAGCATCTATGGTTTTTTCTACTACTAGTCGCCGCTCCAAGAT |
| | HC | GGGTGCTGTCTCAGGTGCAGCTGGTGCAGAGCGGAGCCGAAGT |
| | (with leader | CAAGAAGCCTGGAGCTTCCGTCAAGGTGTCCTGCAAGGCCTCTG |
| | sequence) | GCTATACCTTTACCGGCTACTATATGCACTGGGTGCGGCAGGCCC |
| | | CTGGCCAAGGCCTGGAATGGATGGGCTGGATCAACCCTGATTCT |
| | | GGCGGCACCAACTACGCCCAGAAGTTTCAGGGAAGAGTGACCAT |
| | | GACAAGAGATACCTCTATCTCCACCGCCTACATGGAGCTGAACAG |
| | | ACTGAGATCTGACGACACCGCCGTGTACTACTGTGCTCGGGACC |
| | | AGCCCCTGGGATACTGCACCAATGGCGTGTGCTCCTACTTCGACT |
| | | ACTGGGGCCAAGGGACCTTGGTGACAGTGTCCTCCGCCTCTACC |
| | | AAGGGCCCTTCTGTGTTCCCTCTGGCCCCCTGTTCTCGGTCTACA |
| | | TCTGAGTCCACCGCTGCTCTGGGCTGCCTGGTGAAGGATTACTTT |
| | | CCTGAGCCTGTGACCGTGTCTTGGAACTCCGGCGCTCTGACCTC |
| | | CGGCGTGCACACCTTCCCTGCCGTGCTCCAATCTAGCGGGCTGT |
| | | ACTCTCTGTCCTCCGTCGTGACCGTTCCTTCCTCCAACTTCGGCA |
| | | CCCAGACCTACACCTGCAACGTGGATCACAAGCCCTCCAACACCA |
| | | AAGTGGACAAGACCGTCGAGCGGAAGTGCTGTGTGGAATGTCCT |
| | | CCTTGCCCAGCTCCTCCCGTGGCTGGCCCATCTGTGTTTCTGTTC |
| | | CCACCTAAGCCTAAGGACACACTGATGATCTCCCGCACACCTGAA |
| | | GTGACATGCGTGGTGGTGGACGTCTCCCATGAAGATCCTGAAGT |
| | | GCAGTTCAACTGGTACGTGGATGGCGTGGAGGTGCACAACGCCA |
| | | AGACCAAGCCTAGAGAGGAACAGTTCAACAGCACCTTCCGGGTG |
| | | GTCTCCGTGCTGACAGTTGTGCACCAGGACTGGCTGAACGGCAA |
| | | AGAGTACAAGTGCAAAGTGTCTAACAAGGGCCTGCCTGCCCCTAT |
| | | CGAGAAGACCATCAGCAAAACCAAGGGCCAGCCCAGAGAACCTC |
| | | AGGTGTACACCCTGCCTCCTTCTAGGGAAGAGATGACCAAGAATC |
| | | AGGTGTCTCTGACCTGTCTGGTCAAGGGCTTCTACCCCTCCGACA |
| | | TCGCCGTGGAGTGGGAGTCCAACGGACAGCCTGAGAACAACTAC |
| | | AAGACAACCCCTCCTATGCTGGACAGCGACGGCAGCTTCTTCCTG |
| | | TACAGCAAGCTGACAGTGGACAAGAGCCGGTGGCAGCAGGGCAA |
| | | TGTGTTCAGCTGCTCCGTGATGCACGAGGCCCTGCACAACCACTA |
| | | CACACAGAAATCTCTGAGCCTCAGCCCCGGCAAGGGCGGTGGCG |
| | | GGTCCGGCGGCGGTGGCTCTGGCGGCGGCGGCAGCGATGTGGT |
| | | CATGACCCAGTCCCCTGACTCCCTGGCTGTGTCCCTGGGCGAGA |
| | | GAGCTACGATCAACTGTAAGTCCTCTCAGTCCATCGTGCATCGGA |
| | | ACGGCAACACCTACCTGGAATGGTACCAGCAAAAACCCGGCCAG |
| | | CCTCCCAAGCTGCTGATCTACAAGGTGTCCAACCGGTTCTCTGGC |
| | | GTGCCCGACAGATTCTCTGGCTCTGGCTCTGGCACCGACTTCAC |
| | | CCTGACCATTAGCTCACTGCAGGCCGAGGACGTAGCCGTGTACT |
| | | ACTGCTTCCAGGGCTCTCACGTGCCTTATACCTTTGGCGGCGGCA |
| | | CCAAACTCGAGATCAAGGGCGGTGGCGGCTCCGGAGGAGGCGG |
| | | AAGCGGCGGCGGAGGCTCCCAAGTGCAGCTGGTGCAGTCCGGC |
| | | GCTGAGGTGAAGAAACCTGGTGCTTCCGTGAAGGTGTCTTGCAA |
| | | GGCCTCCGGCTACTCCTTCACCACCTACACCATCCACTGGGTCAG |
| | | ACAGGCTCCAGGCCAGGGACTGGAGTGGATCGGCTACATCAATC |
| | | CTAGCTCTGGTTATGCCGCCTACAACCAGAAGTTCCAGGGCAGA |
| | | GTGACACTGACCGCCGATAAGTCCACTTCTACAGCTTATATGGAA |
| | | CTGTCTTCTTTGCGGTCCGAGGACACCGCAGTGTACTACTGCGCC |
| | | AGAGAGAAGTTCTACGGCTACGACTACTGGGGCCAGGGCACCAC |
| | | CGTGACCGTGTCCTCC |
| 183 | DNA BEAT1a4 | ATGGTGCTACAAACTCAAGTGTTTATCAGTTTGCTGCTGTGGATCT |
| | LC | CTGGCGCCTACGGCGATATCCAGATGACCCAGTCCCCTTCCTCT |
| | (with leader | GTGTCCGCTTCCGTGGGCGATAGGGTGACCATCACCTGTAGAGC |
| | sequence) | TTCCCAGGGCATCTACTCTTGGCTGGCCTGGTACCAGCAGAAGC |
| | | CTGGCAAGGCTCCTAACCTGCTGATCTATACCGCCTCCACACTGC |
| | | AATCTGGAGTGCCCAGCAGATTCTCCGGCTCCGGCTCTGGCACC |
| | | GACTTCACCCTGACCATCTCCTCTCTGCAGCCTGAGGACTTCGCC |
| | | ACCTACTACTGCCAGCAGGCCAACATCTTCCCTCTGACCTTCGGC |
| | | GGCGGAACAAAGGTGGAAATTAAGCGGACCGTGGCCGCTCCCTC |
| | | TGTCTTTATCTTTCCTCCATCCGACGAGCAGCTGAAAAGCGGCAC |
| | | CGCTAGCGTGGTGTGCCTGCTGAACAACTTCTACCCTAGAGAGG |
| | | CCAAGGTGCAGTGGAAAGTCGACAACGCCCTGCAGTCTGGCAAC |
| | | TCCCAAGAAAGCGTGACCGAGCAGGACTCCAAGGACAGCACCTA |
| | | CTCCCTGTCCTCCACCCTCACCCTGAGCAAGGCTGATTACGAGAA |
| | | GCACAAGGTTTACGCCTGCGAGGTGACACACCAGGGACTGTCTT |
| | | CTCCCGTGACTAAGTCATTCAATCGGGGCGAATGC |
| 184 | DNA BEAT2B3 | ATGAAGCATCTATGGTTTTTTCTACTACTAGTGGCCGCCCCTAGAT |
| | HC | GGGTGCTGTCCGACGTGGTGATGACCCAGTCCCCTGACTCCCTG |
| | (with leader | GCCGTGTCCCTCGGCGAGAGAGCCACCATCAACTGCAAGTCCTC |
| | sequence) | CCAGTCTATCGTGCACAGAAATGGCAACACCTACTTGGAGTGGTA |
| | | CCAGCTGAAGCCTGGACAGCCTCCCAAGCTGCTGATCTACAAGG |
| | | TCTCTAACCGGTTCTCCGGCGTCCCTGATAGATTTTCCGGCTCCG |
| | | GCTCCGGCACCGACTTCACCCTGACCATCTCCAGCCTGCAGGCT |
| | | GAGGACGTGGCTGTGTACTACTGCTTCCAGGGCTCTCACGTGCC |
| | | TTACACTTTCGGCGGCGGCACCAAGCTGGAGATCAAGGGGGGCG |
| | | GCGGCAGCGGCGGAGGCGGTTCTGGCGGCGGCGGCTCTCAGGT |
| | | GCAGCTGGTCCAGTCCGGCGCCGAGGTGAAGAAGCCCGGCGCC |
| | | TCTGTGAAAGTGTCTTGCAAGGCTTCCGGCTACAGCTTCACCACC |
| | | TACACCATGCACTGGGTGCGGCAGGCCCCTGGCCAGGGCCTGG |
| | | AATGGATGGGCTACATCAATCCCAGCTCTGGCTACGCCGCTTACA |
| | | ACCAGAAGTTCCAGGGCAGAGTGACCATGACCGCTGACAAGTCC |
| | | ACATCTACCGCCTACATGGAACTGTCTTCCCTGAGAAGCGAGGAC |
| | | ACCGCTGTGTACTACTGCGCCAGAGAGAAGTTCTACGGCTACGA |
| | | CTATTGGGGACAAGGAACCACCGTGACCGTTTCCAGCGGCGGCG |
| | | GCGGATCTGGAGGCGGAGGCTCTGGCGGCGGCGGCTCCGAAGT |
| | | GCAGCTCGTGCAGTCCGGCGCAGAGGTGAAGAAGCCTGGCGAAT |
| | | CTCTGCGGATCTCCTGCAAGGGCTCCGGGGATAGCTTCACCACA |
| | | TACTGGATCGGCTGGGTCAGACAGATGCCTGGAAAGGGCCTGGA |
| | | ATGGATGGGCATCATCTACCCTGGCGACTCTGACACCATCTACTC |
| | | TCCATCTTTCCAAGGCCAAGTGACCATCTCTGCCGATAAGTCCAT |
| | | CTCTACAGCCTACCTGCAGTGGTCCAGTCTGAAAGCCTCCGATAC |
| | | CGCCATGTACTACTGCACAAGAGGAGATAGAGGCGTGGACTATT |
| | | GGGGCCAGGGCACCCTGGTGACCGTGTCTTCTGCTTCTACCAAA |
| | | GGCCCCTCCGTGTTTCCTCTGGCTCCTTCTTCCAAGTCCACCTCC |
| | | GGCGGTACGGCCGCTCTGGGCTGTCTGGTCAAGGACTACTTCCC |
| | | TGAGCCTGTGACCGTCTCTTGGAACTCTGGAGCTCTGACCTCTGG |
| | | CGTGCATACCTTCCCAGCCGTGCTGCAATCCTCCGGACTGTACTC |
| | | GCTGTCTTCTGTGGTGACAGTGCCCAGCTCTTCTCTGGGAACACA |
| | | GACCTACATCTGCAACGTGAACCACAAGCCCTCGAACACCAAGGT |
| | | GGACAAGAAGGTGGAACCTAAGTCTTGCGACAAAACCCACACCT |
| | | GCCCTCCTTGTCCTGCTCCCGAGGCAGCTGGCGGCCCTAGTGTG |
| | | TTTCTGTTCCCTCCCAAGCCTAAGGACACCTTGATGATCTCTCGC |
| | | ACCCCTGAGGTCACCTGTGTGGTCGTGGACGTGTCCCACGAGGA |
| | | CCCTGAGGTGAAGTTTAACTGGTACGTGGACGGCGTGGAAGTGC |
| | | ACAACGCCAAGACCAAGCCCCGGGAGGAACAGTACAACTCCACC |
| | | TACCGGGTTGTGTCCGTGCTGACAGTGCTGCACCAGGATTGGCT |
| | | GAACGGAAAAGAGTACAAGTGCAAAGTGTCTAACAAGGCCCTGC |
| | | CTGCTCCTATCGAGAAGACAATTAGCAAGGCTAAGGGTCAGCCTC |
| | | GGGAGCCTCAGGTGTATACACTGCCACCTTCCAGGGAAGAGATG |
| | | ACAAAGAACCAAGTGAGCCTGACCTGTCTGGTGAAGGGCTTCTAC |
| | | CCATCCGACATCGCCGTGGAATGGGAGAGTAACGGCCAGCCCGA |
| | | GAACAACTACAAAACCACACCTCCAGTGCTGGACTCCGATGGCTC |
| | | CTTCTTCCTGTATTCTAAGCTGACCGTGGACAAGTCCCGGTGGCA |
| | | GCAGGGCAACGTGTTCTCCTGCTCCGTGATGCATGAAGCCCTGC |
| | | ACAATCACTACACCCAGAAATCTCTGTCCCTGTCTCCTGGCAAG |
| 185 | DNA BEAT2B3 | ATGGTGCTACAAACTCAAGTGTTTATCAGTCTCCTGCTGTGGATCT |
| | LC | CTGGAGCCTATGGCGACATCCAGATGACCCAGTCTCCTTCCAGC |
| | (with leader | CTGTCTGCCTCCGTGGGCGATAGAGTGACCATCACCTGCAGAGC |
| | sequence) | CTCTCAGGGAATCTCCAGGTGGCTGGCCTGGTACCAGCAGAAGC |
| | | CTGAAAAAGCTCCTAAGTCCCTGATCTACGCTGCTTCCTCTCTGC |
| | | AATCTGGCGTGCCTTCTCGGTTCTCCGGATCTGGCTCTGGCACC |
| | | GACTTCACCCTGACAATCAGCGGCCTGCAGCCTGAGGATTTTGCT |
| | | ACCTACTACTGCCAGCAGTACAACTCCTACCCCAGAACCTTTGGC |
| | | CAGGGCACCAAAGTGGAAATCAAGCGGACCGTGGCCGCTCCATC |
| | | CGTGTTCATCTTCCCTCCCAGCGACGAGCAGCTGAAGTCTGGCA |
| | | CAGCTTCTGTCGTGTGCCTGCTGAACAACTTCTACCCTAGAGAGG |
| | | CCAAGGTGCAGTGGAAGGTGGATAACGCCCTGCAGTCCGGCAAT |
| | | TCCCAAGAGTCCGTGACTGAGCAGGACTCCAAGGACTCCACCTA |
| | | CTCCCTGAGCTCTACCCTGACACTGTCCAAGGCCGACTACGAGAA |
| | | GCACAAGGTGTACGCCTGTGAAGTGACCCACCAGGGCCTGAGTA |
| | | GCCCCGTCACCAAGTCCTTCAACCGGGGCGAGTGC |
| 186 | Human macrophage | LLDTRQFLIYLEDTKRCVDA |
| | B11 epitope | |

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The present inventors have developed bispecific polypeptides, for example antibodies, that bind to CD40 on an antigen presenting cell and a FLAG tag present on a molecule expressed on an immune cell (such as a T cell), or that bind to CD206 on an antigen presenting cell and a FLAG tag present on a molecule expressed on an immune cell (such as a T cell). These bispecific polypeptides allow engagement of immune cells expressing engineered TCRs (e.g. CAR T cells) with antigen presenting cells (APCs) thereby enhancing immune cell efficacy in vitro and in vivo with a high degree of tumour inhibition without toxicity, while reducing the effective dose of the immune cells expressing engineered TCRs (e.g. CAR T cells) required to achieve a therapeutic effect. Importantly, the immune cells expressing engineered TCRs (e.g. CAR T cells) receive activation and proliferation signals in lymphoid tissue, away from the immunosuppressive tumour microenvironment.

### Bispecific Polypeptides

As used herein, the term “bispecific polypeptide” means a polypeptide that can specifically bind two different target antigens simultaneously. The bispecific polypeptides described herein comprise two structurally distinct binding proteins or domains (i.e., regions), each of which specifically binds to a single target antigen. Bispecific polypeptides can be used to bind to a target antigen on an APC (e.g. CD40 or CD206) and a different target antigen on an immune cell that expresses an engineered TCR (e.g. a CAR containing a FLAG tag). Further, the bispecific polypeptides of the invention can be used to bind to a target antigen on an APC and to a different target antigen on an engineered TCR (e.g. a CAR) expressed by an immune cell. Bispecific polypeptides can include polypeptide sequences (i.e., domains) of one of more antibodies or antibody fragments (e.g., one or more scFvs or IgGs). In another embodiment, the bispecific polypeptides can include polypeptide sequences of one or more ligands.

In any embodiment, the bispecific polypeptide is a tandem single-chain variable fragment antibody (taFv) having a first scFv and a second scFv. In alternative embodiments, the bispecific polypeptide may be in the form of a fusion protein comprising an immunoglobulin protein for binding to a first antigen, and one or more scFvs fused thereto, for binding to a second antigen. As further described herein, it will be appreciated that the bispecific polypeptides of the invention may be in various different formats wherein the first and second antigen binding proteins comprise antigen binding domains, and wherein the proteins may be in any one of the various formats derived from antibodies.

The bispecific polypeptides described herein may be variously referred to as “Bispecific Engagers of APCs and T cells” or “BEATs”.

“Polypeptide”, “peptide”, “protein” and “proteinaceous molecule” are used interchangeably herein to refer to molecular comprising or consisting of a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers.

The term “amino acid” as used herein refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basis chemical structure as a naturally occurring amino acid, e.g., a carbon that is bound to a hydrogen, a carboxyl group, an amino group, and an R group. Such analogs may have modified R groups (e.g., norleucine) or modified peptide backbones. Amino acid mimetics refers to chemical compounds that have a structure that is different from the general chemical structure of an amino acid, but that functions similarly to a naturally occurring amino acid.

Amino acids may be referred to herein by their commonly used full name (e.g., cysteine), their commonly known three letter symbols (e.g., Cys), or by the one-letter symbols recommended by the IUPAC-IUB Biochemical Nomenclature Commission (e.g., C). Nucleotides, likewise, may be referred to by their commonly accepted single-letter codes.

The term “antigen” as used herein refers to a molecule bound by an “antibody”, “antibody fragment” or a “bispecific polypeptide”. Antigens may be proteins recognized by immunoglobulins, in which case the sites on the proteins bound by the immunoglobulins are referred to as “epitopes”. In an embodiment, the antigen can be CD40, CD206 or a FLAG tag. In another embodiment, the antigen is a tag on an engineered TCR (eg a CAR) expressed by an immune cell.

The term “antigen presenting cell” or “APC” as used herein may be a professional antigen presenting cell (e.g., a dendritic cell, macrophage, B-cell, epithelial cell, etc.) or a non-professional antigen presenting cell (e.g., a fibroblast, thymic epithelial cell, thyroid epithelial cell, glial cell, pancreatic beta cell, vascular endothelial cell, etc.). In a preferred embodiment, the APC is an endogenous professional antigen binding cell, (preferably a dendritic cell or macrophage) and which is not a tumor cell.

Examples of APC as described herein, include but are not limited to, dendritic cells (DCs), peripheral blood mononuclear cells (PBMC), monocytes (such as THP-1), B lymphoblastoid cells (such as C1R.A2, 1518 B-LCL) and monocyte-derived dendritic cells (moDCs). In any embodiment, the first antigen binding protein specifically binds to an antigen expressed on an APC (such as a DC or moDC), wherein the antigen is selected from CD40 and CD206. In preferred embodiments, the antigen (CD40 or CD206) specifically bound by the first antigen binding protein is not a tumor-associated antigen. In other words, it will be understood that in the context of use of the bispecific polypeptides of the invention, the intention is that the first antigen binding polypeptide be for binding to CD40 or CD206 on non-tumour, endogenous professional antigen presenting cells, such as DCs, moDCs.

The term “antigen presentation” refers to the process by which APCs capture antigens and enables their recognition by T-cells, e.g., as a component of an MHC-I and/or MHC-II conjugate.

“MHC molecules” include two types of molecules, MHC class I and MHC class II. MHC class I molecules present antigen to specific CD8+ T cells and MHC class II molecules present antigen to specific CD4+ T cells. Antigens delivered exogenously to APCs are processed primarily for association with MHC class II. In contrast, antigens delivered endogenously to APCs are processed primarily for association with MHC class I.

In any embodiment, the second antigen binding protein specifically binds to an antigen on an immune cell expressing an engineered TCR (eg a CAR) or similar receptor. In further embodiments, the second antigen binding protein of the bispecific polypeptides of the invention, may bind to an antigen on the immune cell, wherein the antigen is not part of the engineered TCR, but is an antigen present on the cell surface of the immune cell.

In certain embodiments, the immune cell is an engineered T-cell or NK cell, typically, wherein the antigen on the engineered T-cell or NK cell is part of an engineered TCR (eg a CAR). In accordance with these embodiments, the second antigen binding protein of the bispecific polypeptide of the invention may bind to an extracellular portion of the engineered TCR. The second antigen binding protein may bind to the antigen-binding domain of the engineered TCR, or it may bind to an extracellular region of the engineered TCR that is not involved in antigen binding.

In certain embodiments, the engineered TCR (eg a CAR) present on an immune cell can also comprise additional amino acids or molecules for binding with the second antigen binding protein. As is known in the art, engineered TCR (eg a CAR) constructs may be designed to include a “tag”, which is typically a short amino acid sequence that is specifically recognized by an antibody. In some embodiments, the immune cell is a T-cell or a NK cell engineered to express an engineered TCR (eg a CAR) which includes a tag. In the context of such embodiments, the second antigen binding protein of the bispecific polypeptide may bind to the tag.

In any embodiment, the tag is a FLAG tag. Therefore, the second antigen binding protein is capable of specifically binding to a FLAG tag or variant thereof (such as defined in SEQ ID NOs: 80 and 99 or as otherwise defined herein). The second antigen binding protein is preferably capable of specifically binding to protein domains comprising multiples of the FLAG tag sequences (eg 2×FLAG, 3×FLAG etc).

In any embodiment, the second antigen binding protein is for specifically binding to a peptide tag that contains or consists of the sequence DYK, preferably the sequence DYKD (SEQ ID NO: 80). In addition to these sequences, other amino acids can be present, preferably hydrophilic amino acids for example R (Arg), D (Asp), E (Glu) and K (Lys) and/or amino acids with aromatic side chains for example Y (Tyr), F (Phe), H (His) and W (Trp).

In a preferred embodiment, the second antigen binding protein of the bispecific protein of the invention are capable of specifically binding to a FLAG-tag that contains or consists of the sequence GDYKDDDDKG (SEQ ID NO: 98), DYKDDDDK (SEQ ID NO: 99), MDYKDDDDK (SEQ ID NO: 100), DFKDDDK (SEQ ID NO: 101), DYKAFDNL (SEQ ID NO: 102), DYKDHDG (SEQ ID NO: 103), MDFKDDDDK (SEQ ID NO: 104), MDYKAFDNL (SEQ ID NO: 105), DYKDHDI (SEQ ID NO: 106), DYKDH (SEQ ID NO: 107), DYKDD (SEQ ID NO: 108), DYKDHD (SEQ ID NO: 109) and/or DYKDDD (SEQ ID NO: 110). The most preferred sequence is DYKDDDDK (SEQ ID NO: 99).

As used herein, the term FLAG-tag also refers to modified FLAG-tags, which are derived from the FLAG-tags described above, especially the tag with the sequence DYKDDDDK, by amino acid insertion, deletion or substitution.

Reference herein to a protein or antibody that “binds to” FLAG tag provides literal support for a protein or antibody that “binds specifically to” or “specifically binds to” a FLAG tag.

In any embodiment, the FLAG tag is present at the N-terminus, the C-terminus or within the protein to which is it intended to bind.

In certain embodiments, the immune cell is a T-cell or a NK cell engineered to express an engineered TCR (e.g. a CAR) and the bispecific polypeptide binds to an extracellular part of the engineered TCR. The skilled person will be familiar with the general architecture and uses of engineered TCR (e.g. a CAR), and more specifically CAR T and CAR NK cells. For example, and as is known in the art, a CAR is a cell-surface receptor comprising an extracellular domain, a transmembrane domain and a cytoplasmic domain in a combination that is not naturally found in a single protein. The extracellular domain comprises an antigen-binding domain, which may be an antibody or antibody fragment. The antibody or antibody fragment may be a human antibody or fragment, humanized antibody or fragment or a non-human antibody or fragment. Typically, the antigen-binding domain of the CAR is an antibody fragment, such as a Fab or scFv. Most typically, the antigen-binding domain of the CAR is a scFv. The extracellular domain also typically comprises a spacer (or hinge) region linking the antigen-binding domain to the transmembrane domain. The spacer region of the CAR may be derived from an immunoglobulin, such as IgG1 or IgG4, or it may be derived from alterative cell-surface proteins, including, but not limited to, CD4, CD8, or CD28.

The terms “Chimeric Antigen Receptor” or “CAR” as used herein mean a recombinant polypeptide comprising an antigen binding domain that is linked to at least one intracellular signaling domain. The antigen binding domain of a CAR is a functional portion of the CAR that specifically binds to (i.e., specifically targets) an antigen expressed on a cancer cell (i.e., a “tumour-associated antigen”). Examples of tumour-associated antigens are known to persons skilled in the art, illustrative examples of which include Mesothelin, Her2, CEA, FBP, CD19 and BCMA.

As used herein “tumour-associated antigen” refers to an antigen that is expressed by cancer cells. A tumour-associated antigen may or may not be expressed by non-tumour cells. When a tumour-associated antigen is not expressed by non-tumour cells (i.e., when it is unique to tumour cells) it may also be referred to as a “tumour-specific antigen.” When a tumour-associated antigen is not unique to a tumour cell, it is also expressed on a non-tumour cell under conditions that fail to induce a state of immunologic tolerance to the antigen. The expression of the antigen on the tumour may occur under conditions that enable the immune system to respond to the antigen. Tumour-associated antigens may be antigens that are expressed on non-tumour cells during fetal development when the immune system is immature and unable to respond, or they may be antigens that are normally present at low levels on normal cells but which are expressed at much higher levels on tumour cells. Those tumour-associated antigens of greatest clinical interest are differentially expressed compared to the corresponding non-tumour tissue and allow for a preferential recognition of tumour cells by specific T-cells or immunoglobulins.

As will be clear from the remainder of the disclosure, the first and second antigen binding proteins of the bispecific polypeptides of the invention are typically in the form of an antibody or an antibody fragment. The term “antibody” as used herein broadly refers to any immunoglobulin (Ig) molecule comprised of four polypeptide chains, two heavy (H) chains and two light (L) chains. Also contemplated herein are bispecific polypeptides comprising antigen-binding fragments, mutants, variants, and derivatives thereof, which retain the essential epitope binding features of the antibody molecule. Such mutants, variants, and derivatives will be known to persons skilled in the art, illustrative examples of which are described elsewhere herein.

An antibody heavy chain will typically comprise a heavy chain variable region (HCVR or VH) and a heavy chain constant region. The heavy chain constant region is typically comprised of three domains, CH1, CH2 and CH3. A light chain will typically comprise a light chain variable region (LCVR or VL) and a light chain constant region, CL. The VH and VL regions can be further subdivided into regions of hypervariability, also known as complementary determining regions (CDR), interspersed with framework regions (FR). Each VH and VL is typically comprised of three CDs and four FR, arranged from amino-terminus to carboxy-terminus in the following order: FR1, CDR1, FR2, CDR2, FR3, CDR3 and FR4. Immunoglobulin molecules can be of any type (e.g. IgG, IgE, IgM, IgD, IgA and IgY), class (e.g. IgG1, IgG2, IgG3, IgG4, IgA1 and IgA2) or subclass.

The term “antigen-binding fragment” or “antibody fragment”, as used herein, means one or more fragments of an antibody that retain the ability to specifically bind to the target antigen. Illustrative examples of antigen-binding fragments include (i) a Fab fragment, a monovalent fragment consisting of the VL, VH, CL and CH1 domains; (ii) a F(ab′)₂ fragment, a F(ab′)₂ fragment, a bivalent fragment comprising two Fab fragments linked by a disulfide bridge at the hinge region; (iii) a Fd fragment consisting of the VH and CH1 domains; (iv) a single-chain variable fragment (scFv) consisting of the VL and VH domains of a single arm of an antibody, (v) a dAb fragment (Ward et al. 1989, Nature, 341: 544-6), which comprises a single variable domain; and (vi) an isolated CDR.

In any embodiment of the first or second aspects, the first antigen binding protein and the second antigen binding protein are different antibody or antibody fragments. For example, the first antigen binding protein may be in an oligomeric format whereby 2 polypeptide chains are linked or bonded, and the second antigen binding protein may be a single polypeptide chain. For example, the first antigen binding protein may be in an IgG format (e.g. with a heavy chain and light chain linked or bonded) and the second antigen binding protein may be in a scFv format (e.g. a single contiguous polypeptide chain).

In any embodiment of the first or second aspects, the first antigen binding protein may comprise or consist of a VH and VL as defined herein and in N to C terminal order the VH and VL may be arrange as VL-VH or VH-VL.

In any embodiment of the first or second aspects, the second antigen binding protein may comprise or consist of a VH and VL as defined herein and in N to C terminal order the VH and VL may be arrange as VL-VH or VH-VL.

In any embodiment of the first or second aspects, the second antigen binding protein may be linked to the N terminus of the first antigen binding protein or the N terminus of an antigen binding domain of the first antigen binding protein. For example, the second antigen binding protein may be linked to the N terminus of a VH or VL of the first antigen binding protein. In one embodiment, the second antigen binding protein is linked to the N terminus of the first antigen binding protein or an antigen binding domain of the first antigen binding protein via a linker, preferably a linker described herein.

In any embodiment of the first or second aspects, the second antigen binding protein may be linked to the C terminus of the first antigen binding protein or the C terminus of an antigen binding domain of the first antigen binding protein. For example, the second antigen binding protein may be linked to the C terminus of a VH or VL of the first antigen binding protein. In one embodiment, the second antigen binding protein is linked to the C terminus of the first antigen binding protein or an antigen binding domain of the first antigen binding protein via a linker, preferably a linker described herein.

The term “sequence identity” or “sequence homology” as used herein refers to the subunit sequence identity between two polymeric molecules, e.g., between two nucleic acid molecules, such as two DNA molecules or two RNA molecules, or between two polypeptide molecules. When a subunit position in both of the two molecules is occupied by the same monomeric subunit, e.g., if a position in each two DNA molecules is occupied by adenine, then they are homologous or identical at that position. The homology between two sequences is a direct function of the number of matching or homologous positions, e.g., if half (e.g., five positions in a polymer ten subunits in length) of the positions in two sequences are homologous, the two sequences are 50% homologous; if 90% of the positions (i.e., 9 of 10), are matched or homologous, the two sequences are 90% homologous.

### Heterodimerisation

The “CH3 domain” comprises the stretch of residues C-terminal to a CH2 domain in an Fc region (i.e. from an amino acid residue at about position 341 to an amino acid residue at about position 447 of an IgG). Within a bispecific polypeptide (e.g. antibody) according to the invention described herein, one respective CH3 domain may be arranged at the C-terminus of the VH3 and VL3 domain of the third binding site. The “CH3 domains” as described herein may be variant CH3 domains, wherein the amino acid sequence of the natural CH3 domain was subjected to at least one distinct amino acid substitution (i.e. modification of the amino acid sequence of the CH3 domain) in order to promote dimerisation of the two CH3 domains facing each other within a multispecific antibody.

Several approaches for CH3-modifications in order to support heterodimerization have been described, for example in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291, the disclosures of which are hereby incorporated by reference in their entirety.

Typically, in the heterodimerisation approaches known in the art, the CH3 domain of one heavy chain and the CH3 domain of the other heavy chain are both engineered in a complementary manner so that the heavy chain comprising one engineered CH3 domain can no longer homodimerise with another heavy chain of the same structure. Thereby the heavy chain comprising one engineered CH3 domain is forced to heterodimerise with the other heavy chain comprising the CH3 domain, which is engineered in a complementary manner.

One heterodimerisation approach known in the art is the so-called “knobs-into-holes” technology, which is described in detail providing several examples in e.g. WO 96/027011, Ridgway, J. B., et al., Protein Eng. 9 (1996) 617-621; Merchant, A. M., et al., Nat. Biotechnol. 16 (1998) 677-681; and WO 98/050431, the disclosure of which are hereby incorporated by reference in their entirety. In the “knobs-into-holes” technology, within the interface formed between two CH3 domains in the tertiary structure of the antibody, particular amino acids on each CH3 domain are engineered to produce a protuberance (“knob”) in one of the CH3 domains and a cavity (“hole”) in the other one of the CH3 domains, respectively. In the tertiary structure of the multispecific antibody the introduced protuberance in the one CH3 domain is positionable in the introduced cavity in the other CH3 domain.

In any embodiment of the first or second aspect, the bispecific polypeptide may comprise a CH3 domain comprising an amino acid substitution at T366 or Y407 substitution, for example T366W and Y407V substitutions (numbering according to Kabat).

In any embodiment of the first or second aspect, the bispecific polypeptide may comprise a CH3 domain comprising one or more amino acid substitutions at T366, L368, Y407, S354 and Y349. In preferred examples the CH3 domain may comprise an amino acid substitution of:
(a) T336W;
(b) T366S, L368A and Y407V;
(c) Y407T; or
(d) T336Y

In any embodiment of the above, the bispecific polypeptide may comprise two CH3 domains, wherein:
the first CH3 domain comprises an amino acid substitution of T336W and the second CH3 domain comprises the amino acid substitutions of T366S, L368A and Y407V; or
the first CH3 domain comprises an amino acid substitution of Y407T and the second CH3 domain comprises an amino acid substitutions of T336Y.

In addition or alternatively to engineering the CH3 domains as described herein, the CH3 domain may comprise additional cysteine substitutions to provide additional interchain disulfide bridge stabilizes the heterodimers, for example S354C and/or Y349C substitutions (numbering according to Kabat) (Atwell, S., et al., J. Mol. Biol. 270 (1997) 26-35; Merchant, A. M., et al., Nature Biotech. 16 (1998) 677-681).

### Linkers

The bispecific polypeptides of the invention can include a linker sequence that links the first antigen binding protein and the second antigen binding protein. Alternatively, or in addition, a linker sequence may link one or more antigen binding domains within the first antigen binding protein or one or more antigen binding domains within second antigen binding protein. The linkers may, for example, function to join two domains of an antigen binding protein (such as the VH and VL of an scFv or diabody), or they may function to join two antigen binding proteins together (such as two or more Fabs or sdAbs), or they may function to join an antigen binding protein to a scaffold. In some embodiments, the bispecific polypeptide may comprise multiple linkers (i.e., two or more), for example, one or more scFvs linked to a scaffold may comprise a linker joining the VH and VL of the scFv and a linker joining the scFv to the scaffold. Appropriate linkers are known in the art and can be readily selected by the skilled artisan based on the intended use of the linker (see, for example, Müller & Kontermann, “Bispecific Antibodies” in Handbook of Therapeutic Antibodies, Wiley-VCH Verlag GmbH & Co. 2014).

Useful linkers include glycine-serine (GlySer) linkers, which are well-known in the art and comprise glycine and serine units combined in various orders. Examples include, but are not limited to, (GS), (GSGGS)ₙ (SEQ ID NO: 187), (GGGS)ₙ (SEQ ID NO: 188) and (GGGGS)ₙ (SEQ ID NO: 189), where n is an integer of at least one, typically an integer between 1 and about 10, for example, between 1 and about 8, between 1 and about 6, or between 1 and about 5.

Other useful linkers include sequences derived from immunoglobulin hinge sequences. The linker may comprise all or part of a hinge sequence from any one of the four IgG classes and may optionally include additional sequences. For example, the linker may include a portion of an immunoglobulin hinge sequence and a glycine-serine sequence. A non-limiting example is a linker that includes approximately the first 15 residues of the IgG1 hinge followed by a GlySer linker sequence, such as those described above, that is about 10 amino acids in length.

The length of the linker will vary depending on its application. Appropriate linker lengths can be readily selected by the skilled person. For example, when the linker is to connect the VH and VL domains of an scFv, the linker is typically between about 5 and about 20 amino acids in length, for example, between about 10 and about 20 amino acid in length, or between about 15 and about 20 amino acids in length. When the linker is to connect the VH and VL domains of a diabody, the linker should be short enough to prevent association of these two domains within the same chain. For example, the linker may be between about 2 and about 12 amino acids in length, such as, between about 3 and about 10 amino acids in length, or about 5 amino acids in length.

In some embodiments, when the linker is to connect two Fab fragments, the linker may be selected such that it maintains the relative spatial conformation of the paratopes of a F(ab′) fragment, and is capable of forming a covalent bond equivalent to the disulphide bond in the core hinge of IgG. In this context, suitable linkers include IgG hinge regions such as, for example those from IgG1, IgG2 or IgG4. Modified versions of these exemplary linkers can also be used. For example, modifications to improve the stability of the IgG4 hinge are known in the art (see for example, Labrijn et al. 2009, Nature Biotechnology, 27: 767-771).

The linker may comprise a sequence of amino acid residues joining the first and second antigen binding proteins. Alternatively, the first and second antigen binding proteins may be linked via chemical conjugation (for example, to form a bis-aryl conjugate between the domains). Examples of suitable methods for chemical conjugation of binding domains are known in the art. Such methods include the use of succinimidyl compound modification of primary amines present on lysine residues, as used in TriLink Technologies bioconjugation reagents. In an embodiment, if the first and/or second antigen binding proteins are scFvs, the bispecific polypeptide comprises a linker sequence between the VH and VL domains of the scFvs. Suitable linker sequences would be known to persons skilled in the art, illustrative examples of which include relatively flexible and hydrophilic amino acid residues. In an embodiment, the linker sequence is selected from the group consisting of SEQ ID NO: 64 or 65 or an amino acid sequence having at least 70% identity thereto, preferably at least 71%, preferably at least 72%, preferably at least 73%, preferably at least 74%, preferably at least 75%, preferably at least 76%, preferably at least 77%, preferably at least 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, or more preferably at least 99% sequence identity to SEQ ID NO: 64 or 65.

An antibody or antibody fragment can contain additional amino acids or molecules for purification or identification. For example, the antibody can contain an epitope or affinity tag. Illustrative examples of such epitopes or affinity tags include, peptide tags (e.g., FLAG-tag, HA-tag, His-tag, Myc-tag, S-tag, SBP-tag, Strep-tag, eXact-tag) and protein tags (e.g., GST-tag, MBP-tag, GFP-tag). In an embodiment, the epitope or affinity tag is a His-tag.

### Nucleic Acids and Vectors

In another aspect disclosed herein, there is provided a nucleic acid encoding the bispecific polypeptide described herein. In this embodiment, the nucleic acid may encode one or more of the amino acid sequences recited in Table 1. In yet another aspect there is provided a cell comprising the vector described herein.

The terms “polynucleotide”, “polynucleotide sequence”, “nucleotide sequence”, “nucleic acid” or “nucleic acid sequence” as used interchangeably herein to designate mRNA, RNA, cRNA, cDNA or DNA. The term typically refers to polymeric form of nucleotides of at least 10 bases in length, either ribonucleotides or deoxynucleotides or a modified form or either type of nucleotide. The term includes single and double stranded forms of RNA and DNA.

As used herein, the term “gene” includes a nucleic acid molecule capable of being used to produce mRNA optionally with the addition of elements to assist in this process. Genes may or may not be capable of being used to produce a functional protein. Genes can include both coding and non-coding regions (e.g., introns, regulatory elements, promoters, enhancers, termination sequences and 5′ and 3′ untranslated regions).

The term “transgene” is used herein to describe genetic material that has been or is about to be artificially introduced into a genome of a host organism and that is transmitted to the progeny of that host. In some embodiments, it confers a desired property to a T cell which it is introduced, or otherwise leads to a desired therapeutic outcome.

As used herein, the terms “encode,” “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

In any embodiment, a nucleic acid sequence encoding the bispecific polypeptides of the invention, may have one or more polynucleotide sequences that encodes or is capable of encoding a polypeptide selected from the group consisting SEQ ID NOs: 1, 2, 17 to 32, 33, 34, 49 to 63, 64 to 67, 71 to 74 and 76 to 79.

In any embodiment, a nucleic acid sequence encoding the bispecific polypeptides of the invention, may have one or more polynucleotide sequences selected from the group consisting of SEQ ID NOs: 178 to 185.

In another aspect, there is provided a vector comprising the nucleic acid described herein operably linked to a regulatory sequence.

The terms “regulatory element” or “regulatory sequence” refer to nucleic acid sequences (e.g., DNA) necessary for expression of an operably linked coding sequence in a particular cell. The regulatory sequences that are suitable for eukaryotic cells include promoters, polyadenylation signals, transcriptional enhancers, translational enhancers, leader or trailing sequences that modulate mRNA stability, as well as targeting sequences that target a product encoded by a transcribed polynucleotide to an intracellular compartment within a cell or to the extracellular environment.

Typically, the regulatory sequences include, but are not limited to, a promoter sequence, a 5′ non-coding region, a cis-regulatory region such as a functional binding site for transcriptional regulatory protein or translational regulatory protein, an upstream open reading frame, ribosomal-binding sequences, transcriptional start site, translational start site, and/or nucleotide sequence which encodes a leader sequence, termination codon, translational stop site and a 3′ non-translated region. Constitutive or inducible promoters as known in the art are contemplated. The promoters may be either naturally occurring promoters, or hybrid promoters that combine elements of more than one promoter.

Promoter sequences contemplated may be native to mammalian cells or may be derived from an alternative source, where the region is functional in the chosen organism. The choice of promoter will differ depending on the intended host cell. For example, promoters which could be used for expression in mammalian cells include the metallothionein promoter, which can be induced in response to heavy metals such as cadmium, the β-actin promoter as well as viral promoters such as the SV40 large T antigen promoter, human cytomegalovirus (CMV) immediate early (IE) promoter, Rous sarcoma virus LTR promoter, the mouse mammary tumour virus LTR promoter, the adenovirus major late promoter (Ad MLP), the herpes simplex virus promoter, and a HPV promoter, particularly the HPV upstream regulatory region (URR), among others. All these promoters are well described in the art and readily available.

Enhancer elements may also be used herein to increase expression levels of the nucleic acid sequence within the vector construct. Examples include the SV40 early gene enhancer, as described for example in Dijkema et al. (1985, EMBO Journal, 4:761), the enhancer/promoter derived from the long terminal repeat (LTR) of the Rous Sarcoma Virus, as described for example in Gorman et al., (1982, Proceedings of the National Academy of Science. USA, 79:6777) and elements derived from human CMV, as described for example in Boshart et al. (1985, Cell, 41:521), such as elements included in the CMV intron A sequence.

The vector construct may also comprise a 3′ non-translated sequence. A 3′ non-translated sequence refers to that portion of a gene comprising a DNA segment that contains a polyadenylation signal and any other regulatory signals capable of effecting mRNA processing or gene expression. The polyadenylation signal is characterised by effecting the addition of polyadenylic acid tracts to the 3′ end of the mRNA precursor. Polyadenylation signals are commonly recognized by the presence of homology to the canonical form 5′ AATAAA-3′ although variations are not uncommon. The 3′ non-translated regulatory DNA sequence preferably includes from about 50 to 1,000 nts and may contain transcriptional and translational termination sequences in addition to a polyadenylation signal and any other regulatory signals capable of effecting mRNA processing or gene expression.

The term “operably connected” or “operably linked” as used herein refers to a juxtaposition wherein the components so described are in a relationship permitting them to function in their intended manner. For example, a regulatory sequence “operably linked” to a coding sequence refers to the positioning and/or orientation of the regulatory sequence relative to the coding sequence to permit expression of the coding sequence under conditions compatible with the regulatory sequence.

As used here the terms “open reading frame” and “ORF” are used interchangeably herein to refer to the amino acid sequence encoded between translation initiation and termination codons of a coding sequence. The terms “initiation codon” (e.g., ATG) and “termination codon” (e.g., TGA, TAA, TAG) refer to a unit of three adjacent nucleotides (‘codon’) in a coding sequence that specifies initiation and chain termination, respectively, of protein synthesis (mRNA translation).

As used herein the term “recombinant” as applied to “nucleic acid molecules”, “polynucleotides” and the like is understood to mean artificial nucleic acid structures (i.e., non-replicating cDNA or RNA; or replicons, self-replicating cDNA or RNA) which can be transcribed and/or translated in the cells as described herein.

Recombinant nucleic acid molecules or polynucleotides may be inserted into a vector. Non-viral vectors such as plasmid expression vectors or viral vectors may be used. The kinds of vectors and the technique of insertion of the nucleic acid construct according to this invention are known in the art, illustrative examples of which include cosmids, plasmids (e.g., naked or contained in liposomes) and viruses (e.g., lentiviruses, retroviruses, adenoviruses, and adeno-associated viruses).

In any embodiment, the vector comprises a polynucleotide sequence encoding one or more polypeptide sequences selected from the group consisting of SEQ ID NO: 1, 2, 17 to 32, 33, 34, 49 to 63, 64 to 67, 71 to 74 and 76 to 79.

In any embodiment, the vector comprises one or more polynucleotide sequences selected from the group consisting of SEQ ID NOs: 178 to 185.

The nucleic acid sequence, polynucleotide or vector construct as described herein comprises a heterologous sequence that does not occur in nature.

In yet another aspect, there is provided a host cell comprising the vector described herein. Suitable host cells would be known to persons skilled in the art, illustrative examples of which include bacterial cells (e.g., E. coli, P. mirabilis), fungal cells (e.g., S. cerevisiae, P. pastoria, T. reesei), plant cells, insect cells (e.g., SF-9, SF21, Hi-5), or mammalian cells. In an embodiment, the host cell is a mammalian cell. Suitable mammalian cells would be known to persons skilled in the art, illustrative examples of which include a CHO or 293T cells. These cells are widely available from commercial suppliers.

### Methods for Producing Bispecific Polypeptides

In another aspect, there is provided a method for producing the bispecific polypeptide described herein, comprising (i) culturing a cell as described herein in a culture medium and under conditions suitable for the expression of the bispecific polypeptide; and (ii) isolating the bispecific polypeptide from the cell or from the culture medium.

The bispecific polypeptides described herein can be produced using any number of expression systems would be known to persons skilled in the art, illustrative examples of which include production in or by bacteria (e.g., E. coli, P. mirabilis), fungi (e.g., S. cerevisiae, P. pastoria, T. reesei), plants or plant cells, insects or insect cells (e.g., SF-9, SF21, Hi-5), or mammalian cells. In an embodiment, the expression system is a mammalian expression system. Suitable mammalian expression systems would be known to persons skilled in the art, illustrative examples of which include a CHO or 293 expression system. These expression systems are widely available from commercial suppliers. In an embodiment, the bispecific polypeptides are produced using a mammalian expression system.

In another aspect, there is provided a method for producing the bispecific polypeptide described herein, comprising combining the first antigen binding protein and the second antigen binding protein under conditions suitable for the formation of a chemically conjugated bispecific polypeptide comprising the first antigen binding protein and the second binding protein.

In a preferred embodiment, the chemically conjugated bispecific polypeptide is formed following succinimidyl compound modification of primary amines present on lysine residues, as described elsewhere herein.

### Pharmaceutical Compositions

In another aspect, there is provided a pharmaceutical composition comprising the bispecific polypeptide described herein and a pharmaceutically acceptable carrier.

The composition described herein may be prepared in a manner known in the art and are those suitable for parenteral administration to mammals, particularly humans, comprising a therapeutically effective amount of the composition with one or more pharmaceutically acceptable carriers or diluents.

The term “pharmaceutically acceptable carrier” as used herein means any suitable carriers, diluents or excipients. These include all aqueous and non-aqueous isotonic sterile injection solutions which may contain anti-oxidants, buffers and solutes, which render the composition isotonic with the blood of the intended recipient; aqueous and non-aqueous sterile suspensions, which may include suspending agents and thickening agents, dispersion media, antifungal and antibacterial agents, isotonic and absorption agents and the like. It will be understood that compositions of the invention may also include other supplementary physiologically active agents.

The carrier is typically pharmaceutically “acceptable” in the sense of being compatible with the other ingredients in the composition and not injurious to the subject. Compositions include those suitable for parenteral administration, including subcutaneous, intramuscular, intravenous and intradermal administration. The compositions may conveniently be presented in unit dosage form and may be prepared by any method well known in the art of pharmacy. Such methods include preparing the carrier for association with the isolated T cells. In general, the compositions are prepared by uniformly and intimately bringing into association any active ingredients with liquid carriers.

In an embodiment, the composition is suitable for parenteral administration. In another embodiment, the composition is suitable for intravenous administration.

Compositions suitable for parenteral administration include aqueous and non-aqueous isotonic sterile injection solutions which may contain anti-oxidants, buffers, bactericides and solutes, which render the composition isotonic with the blood of the intended recipient; and aqueous and non-aqueous sterile suspensions which may include suspending agents and thickening agents.

The present disclosure also contemplates the combination of the composition described herein with other active agents and/or in addition to other treatment regimens or modalities such as radiation therapy or surgery. When the composition described herein is used in combination with known active agents, the combination may be administered either in sequence (either continuously or broken up by periods of no treatment) or concurrently or as an admixture. Suitable anti-cancer agents will be known to persons skilled in the art. Treatment in combination is also contemplated to encompass the treatment with either the composition of the invention followed by a known treatment, or treatment with a known agent followed by treatment with the composition of the invention, for example, as maintenance therapy. For example, in the treatment of cancer it is contemplated that the composition of the present invention may be administered in combination with an alkylating agent (such as mechlorethamine, cyclophosphamide, chlorambucil, ifosfamidecysplatin, or platinum-containing alkylating agents such as cisplatin, carboplatin and oxaliplain), and anti-metabolite (such as a purine or pyrimidine analogue or an anti-folate agent, such as azathioprine and mercaptopurine), an anthracycline (such as daunorubicin, doxorubicin, epirubicin idarubicin, valrubicin, mitoxantrone or anthracycline analog), a plant alkaloid (such as a vinca alkaloid or a taxane, such as vincristine, vinblastine, vinorelbine, vindesine, paclitaxel or doestaxel), a topoisomerase inhibitor (such as a type I or type II topoisomerase inhibitor), a podophyllotoxin (such as etoposide or teniposide), a tyrosine kinase inhibitor (such as imatinib mesylate, nilotinib or dasatinib), an adenosine receptor inhibitor (such as A2aR inhibitors, SCH58261, CPI-444, SYN115, ZM241385, FSPTP or A2BR inhibitors such as PSB-1115), adenosine receptor agonists (such as CCPA, IB-MECA and CI-IB-MECA), a checkpoint inhibitor, including those of the PDL-1:PD-1 axis, nivolumab, pembrolizumab, atezolizumab, BMS-936559, MED14736, MPDL33280A or MSB0010718C), an inhibitor of the CTLA-4 pathway (such as ipilimumab and tremelimumab), an inhibitor of the TIM-3 pathway or an agonist monoclonal antibody that is known to promote T cell function (including anti-OX40, such as MED16469; and anti-4-BB, such as PF-05082566).

In a further aspect, the invention provides a kit or article of manufacture including one or more bispecific polypeptides of the invention, a nucleic acid encoding said bispecific polypeptide and/or pharmaceutical compositions as described above.

In further aspects there is provided a kit for use in a therapeutic application mentioned above, the kit comprising:
(a) a container holding a bispecific polypeptide, nucleic acid, vector or pharmaceutical composition of the invention; and
(b) a label or package insert with instructions for use.

The kit may also comprise one or more active principles or ingredients for treatment of cancer. For example, the kit may also comprise an immune cell expressing a CAR.

The “kit” or “article of manufacture” may comprise a container and a label or package insert on or associated with the container. Suitable containers include, for example, bottles, vials, syringes, blister pack, etc. The containers may be formed from a variety of materials such as glass or plastic. The container holds a therapeutic composition which is effective for treating the condition and may have a sterile access port (e.g., the container may be an intravenous solution bag or a vial having a stopper pierceable by a hypodermic injection needle). The label or package insert indicates that the therapeutic composition is used for treating the condition of choice. In an embodiment, the label or package insert includes instructions for use and indicates that the therapeutic or prophylactic composition can be used to treat a cancer or other condition described herein.

The kit may comprise (a) a therapeutic or prophylactic composition; and (b) a second container with a second active principle or ingredient contained therein. The kit according to this embodiment of the invention may further comprise a package insert indicating the composition and other active principle can be used to treat cancer or condition described herein. Alternatively, or additionally, the kit may further comprise a second (or third) container comprising a pharmaceutically-acceptable buffer, such as bacteriostatic water for injection (BWFI), phosphate-buffered saline, Ringer's solution and dextrose solution. It may further comprise other materials desirable from a commercial and user standpoint, which would be known to persons skilled in the art, suitable examples of which include other buffers, diluents, filters, needles, and syringes.

### Methods of Treatment

In another aspect, there is provided a method for the treatment of cancer comprising co-administering to a subject in need thereof a therapeutically effective amount of: (i) an immune cell expressing an engineered TCR (e.g. CAR); and (ii) a bispecific polypeptide or pharmaceutical composition as described herein, wherein the bispecific polypeptide simultaneously binds to an antigen expressed on an endogenous APC of the subject and an antigen on the engineered TCR (e.g. a CAR) expressed by the immune cell in vivo to stimulate the activation and expansion of the immune cell for the treatment of cancer.

In any method of treatment or use described herein, or other aspect of the invention where a bispecific polypeptide is administered to a subject, that subject may have received or be receiving an immune cell expressing an engineered TCR (e.g. CAR).

In any method of treatment, or other aspect of the invention where a bispecific polypeptide is administered to a subject, that method or other statement further comprises a step of administering an immune cell expressing an engineered TCR (e.g. CAR).

Co-administration of an immune cell expressing a engineered TCR (e.g. a CAR) and a bispecific polypeptide or pharmaceutical composition as described herein may be achieve by formulating the immune cell and the bispecific polypeptide or pharmaceutical composition in the same composition (e.g., for simultaneous co-administration) or they may be formulated as different compositions for sequential administration. By “sequential” administration is meant there is an interval between the administration of the immune cell and the bispecific polypeptide or pharmaceutical composition. The interval between sequential administrations may be seconds, minutes, hours or days. In an embodiment, periodic re-administration of the immune cell and the bispecific polypeptide or pharmaceutical composition may be required to achieve a desired therapeutic effect. Sequential administration may be in any order.

The inventors have surprisingly shown that the bispecific polypeptide described herein stimulates the activation and expansion of immune cell in vivo, thereby improving the efficacy of immune cell therapies, such as CAR T cell therapies. Accordingly, the bispecific polypeptide may be referred to as an “adjuvant”. The term “adjuvant” as used herein refers to a bispecific polypeptide that can increase the magnitude of the immune response elicited by the immune cell expressing a engineered TCR (e.g. a CAR) beyond that which would be expected from the immune cell expressing a engineered TCR alone.

Immune cell activation can be accomplished by providing a primary stimulation signal through, for example, the T cell TCR/CD3 complex or via stimulation of the CD2 surface protein and by providing a secondary co-stimulation signal through an accessory molecule, e.g. CD28 or 4-1BBL. In addition to the primary stimulation signal provided through the TCR/CD3 complex, or via CD2, induction of T cell responses requires a second, costimulatory signal. In particular embodiments, a CD28 binding agent can be used to provide a costimulatory signal. Suitable costimulatory ligands include, but are not limited to, CD7, B7-1 (CD80), B7-2 (CD86), 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, ILT3, ILT4, an agonist or antibody that binds Toll-like receptor, and a ligand that specifically binds with B7-H3.

The terms “expanded”, “expansion”, “expanding”, “propagation” and the like are used interchangeably herein to refer to the increase in the number of immune cells, either prior to administration of the cells to the subject or preparation of a pharmaceutical composition or after administration. Immune cells may be expanded using any cell culture method known in the art. For example, immune cells may be expanded in in vitro tissue culture systems, including liquid culture, monolayers and the like.

The therapeutic regimen for the treatment of cancer can be determined by a person skilled in the art and will typically depend on factors including, but not limited to, the type, size, stage and receptor status of the tumour in addition to the age, weight and general health of the subject. Another determinative factor may be the risk of developing recurrent disease. For instance, for a subject identified as being at high risk or higher risk or developing recurrent disease, a more aggressive therapeutic regimen may be prescribed as compared to a subject who is deemed at a low or lower risk of developing recurrent disease. Similarly, for a subject identified as having a more advanced stage of cancer, for example, stage III or IV disease, a more aggressive therapeutic regimen may be prescribed as compared to a subject that has a less advanced stage of cancer.

The term “cancer” as used herein means any condition associated with aberrant cell proliferation. Such conditions will be known to persons skilled in the art. In an embodiment, the cancer is a solid cancer. In another embodiment, the cancer is a Mesothelin positive cancer. In another embodiment, the cancer does not express CD40 or CD206, or relative to non-cancerous tissue of the same type, CD40 or CD206 is downregulated. In another embodiment, the cancer may express CD40. In another embodiment, the cancer is selected from the group consisting of brain cancer, breast cancer, lung cancer, colon cancer, ovarian cancer, oesophageal cancer, skin cancer, prostate cancer, pancreatic cancer, uterine cancer, gastric cancer, thymic carcinoma and endometrial cancer.

The terms “treat”, “treatment” and “treating” as used herein refers to any and all uses which remedy a condition or symptom, or otherwise prevent, hinder, retard, abrogate or reverse the onset or progression of cancer or other undesirable symptoms in any way whatsoever. Thus, the term “treating” and the like are to be considered in their broadest possible context. For example, treatment does not necessarily imply that a subject is treated until total recovery or cure. In conditions that display or are characterized by multiple symptoms, the treatment need not necessarily remedy, prevent, hinder, retard, abrogate or reverse all of said symptoms, but may remedy, prevent, hinder, retard, abrogate or reverse one or more of said symptoms.

The subject in which cancer is to be treated may be a human or a mammal of economic importance and/or social importance to humans, for instance, carnivores other than humans (e.g., cats and dogs), swine (e.g., pigs, hogs, and wild boars), ruminants (e.g., cattle, oxen, sheep, giraffes, deer, goats, bison, and camels), horses, and birds including those kinds of birds that are endangered, kept in zoos, and fowl, and more particularly domesticated fowl, e.g., poultry, such as turkeys, chickens, ducks, geese, guinea fowl, and the like, as they are of economic importance to humans. The term “subject” does not denote a particular age. Thus, adult, juvenile and newborn subjects are intended to be covered.

The terms “subject”, “individual” and “patient” are used interchangeably herein to refer to any subject to which the present disclosure may be applicable. In an embodiment, the subject is a mammal. In another embodiment, the subject is a human.

The term “therapeutically effective amount” as used herein means the amount of cells when administered to a mammal, in particular a human, in need of such treatment, is sufficient to treat cancer. The precise amount of modified cells to be administered can be determined by a physician with consideration of individual differences in age, weight, tumor size, extent of infection or metastasis, and condition of the subject.

Typically, administration of immune cell (e.g., CAR-T cell) therapies is defined by number of cells per kilogram of body weight. However, because the modified cells will replicate and expand after transfer, the administered cell dose will not resemble the final steady-state number of cells. In an embodiment, a pharmaceutical composition comprising the modified cells of the present invention may be administered at a dosage of 10⁴ to 10⁹ cells/kg body weight. In another embodiment, a pharmaceutical composition comprising the modified cells of the present invention may be administered at a dosage of 10⁵ to 10⁶ cells/kg body weight, including all integer values within those ranges.

Compositions comprising the bispecific polypeptides as described herein can also be administered multiple times at these dosages. The optimal dosage and treatment regimen for a particular subject can be readily determined by one skilled in the art by monitoring the patient for signs of disease and adjusting the treatment accordingly.

In an embodiment, the immune cell is derived from an autologous cell. In another embodiment, the immune cell is derived from an allogeneic cell.

The term “autologous” refers to any material derived from the same individual to whom the material is later to be re-introduced to the individual.

The term “allogeneic” refers to any material derived from a different individual of the same species as the individual to whom the material is introduced. Two or more individuals are said to be allogeneic to one another when the genes at one or more loci are not identical. In some aspects, allogeneic materials from individuals of the same species may be sufficiently genetically distinct to interact antigenically.

### Cell Manufacturing

In order to achieve sufficient therapeutic doses of immune cell compositions, methods for manufacturing of immune cells typically involved one or more rounds of stimulation, activation and/or expansion. In accordance with the methods disclosed herein, immune cells may be stimulated to activate and expand both in vivo and in vitro.

Accordingly, in another aspect disclosed herein, there is provided a method for stimulating the activation and/or expansion of an immune cell in vivo comprising administering to a subject an effective amount of: (i) an immune cell expressing a engineered TCR (e.g. a CAR); and (ii) a bispecific polypeptide, or the pharmaceutical composition as described herein, wherein the bispecific polypeptide simultaneously binds to an antigen expressed on an endogenous APC of the subject and an antigen on the engineered TCR (e.g. a CAR) expressed by the immune cell in vivo.

The in vivo activation and expansion of immune cells as described herein facilitates a reduction in the number of immune cells required for each therapeutic dose of the immune cell.

In another aspect, there is provided a method for stimulating the activation and expansion of an immune cell in vitro comprising culturing an isolated immune cell expressing an engineered TCR (e.g. a CAR) in a culture medium comprising: (i) an APC or APC mimetic; and (ii) a bispecific polypeptide as described herein, wherein the bispecific polypeptide simultaneously binds to an antigen expressed on the APC or APC mimetic and an antigen on the engineered TCR (e.g. a CAR) expressed by the immune cell. In an embodiment, the bispecific polypeptide may bind to an antigen expressed on a tumour cell.

The immune cell manufacturing methods contemplated herein simple and robust culture initiation and activation steps that contribute to a resulting immune cell composition that is a superior therapeutic product. In one embodiment, culture initiation and activation comprises seeding cell populations in a cell culture vessel, e.g., cell culture bag, GREX bioreactor, WAVE bioreactor, etc. and activating immune cells through primary and co-stimulatory immune cell signaling pathways. The cellular compositions may further be cultured in the presence of one or more additional growth factors or cytokines, e.g., IL-2, IL7, and/or IL-15, or any suitable combination thereof.

In another embodiment, activation and expansion of immune cells comprises culturing isolated immune cells with an APC or APC mimetic. In an embodiment, the APC or APC mimetic is selected from the group consisting of a donor-derived APC, a synthetic artificial APC (aAPC), microbeads (Dynabeads) functionalized with activating antibodies for CD3 and CD8, autologous monocyte-derived dentritic cells (moDCs) and scaffolds that mimic APCs.

In particular embodiments, culture initiation comprises seeding a population of cells comprising T cells, e.g., PBMCs, in a cell culture vessel, at a desired density, e.g., 1-5×10⁶ cells/mL in a suitable cell culture medium containing one or more cytokines, primary stimulatory ligands, and co-stimulatory ligands. In another embodiment, the cytokines, stimulatory, and co-stimulatory ligands may be subsequently added to the PBMCs in the cell culture medium.

In one embodiment, the cell culture vessel is a cell culture bag, including but not limited MACS® GMP Cell Expansion Bags, MACS® GMP Cell Differentiation Bags, EXP-Pak™ Cell Expansion Bio-Containers, VueLife™ bags, KryoSure™ bags, KryoVue™ bags, Lifecell® bags, PermaLife™ bags, X-Fold™ bags, Si-Culture™ bags, and VectraCell™ bags, as contemplated elsewhere herein.

In particular embodiments, the cells are seeded in a cell culture vessel comprising a suitable cell culture medium. Illustrative examples of suitable cell culture media include, but are not limited to TCGM; X-VIVO™15 supplemented with 2 mM GlutaMAX™-I, 10 mM HEPES, and 5% human AB serum), CTS™ OpTmizer™ T Cell Expansion SFM (Life Technologies), CTS™ AIM V® Medium (Life Technologies), RPMI 1640, Clicks, DMEM, MEM, a-MEM, F-12, X-Vivo 15 (Lonza), CellGro® Serum-Free Medium (CellGenix), and X-Vivo 20 (Lonza) with added amino acids, sodium pyruvate, and vitamins, either serum-free or supplemented with an appropriate amount of serum (or plasma) or a defined set of hormones, and/or an amount of cytokine(s) sufficient for the growth and expansion of immune cells.

Cell culture media contemplated herein may further comprise one or more factors including, but not limited to serum (e.g., fetal bovine or human serum), interleukin-2 (IL-2), insulin, IFN-γ, IL-4, IL-7, IL-21, GM-CSF, IL-10, IL-12, IL-15, TGF-β, and TNF-α.

In an embodiment, the APC or APC mimetic is selected from the group consisting of a donor-derived APC, a synthetic artificial APC (aAPC), microbeads (Dynabeads) functionalized with activating antibodies for CD3 and CD8, autologous monocyte-derived dentritic cells (moDCs) and scaffolds that mimic APCs.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

The various embodiments enabled herein are further described by the following non-limiting examples.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1—Anti-CD40 Antibody Generation

A human anti-CD40 antibody was obtained. 500 mL HEK293 cultures were transfected with a vector encoding the anti-CD40 antibody. Cultures were harvested by centrifugation at 6 days. An SDS-PAGE gel was completed at 3 days. Cysteine mutations were made (C232 and C127) to serines to confirm expression/aggregation. Strong expression was seen across the board with both the cysteine mutant and non-mutated IgG2 formats.

Clarified supernatants of the IgG2 proteins were purified by Protein A Affinity Chromatography & Buffer exchanged (PBS). Materials were dialysed into PBS buffer pH 7.0. Final concentrations were determined by A280 and yields, purity and endotoxin levels are detailed in the table below:

| | Final | | | | | |
|---|---|---|---|---|---|---|
| | concentrations | Volume of | Estimated | Endotoxin | SDS | Analytical |
| Clone name | (A280) | culture | Yield | amounts | Purity | Purity |
| Selicrelumab IgG2 | 1 mg/L | 500 mL | 170 mg/L | <1 EU/mg | >95% | 97.7% |
| Selicrelumab IgG2 (C232S) | 1 mg/L | 500 mL | 200 mg/L | <1 EU/mg | >95% | 95.5% |
| Selicrelumab IgG2 (C127S) | 1 mg/L | 500 mL | 190 mg/L | <1 EU/mg | >95% | 97.9% |

All IgG2 formats were determined to have high expression, low aggregation and high purity and were suitable for BEAT design.

### Example 2—Humanisation of Chimeric Antibody

A mouse anti-FLAG antibody was obtained. For the purpose of identifying complementarity determining regions (CDRs) and analysing the closest matching germline sequences the IMGT Domain Gap Align tool was used (Ehrenmann F., Kaas Q. and Lefranc M.-P. Nucleic Acids Res., 2010; 38:D301-D307).

Molecular models were built for VH and VL domains based on homology to previously published antibody crystal structures using in-house software. PDB files can be provided on request for viewing in any molecular visualization software. Images have been generated using PyMol (The PyMOL Molecular Graphics System, Version 2.0 Schrödinger, LLC).

Antibody sequences were analysed for specific liabilities based on published protein motifs. Analysis was performed by analysing for the following motifs where X represents any amino acid apart from Proline as described in the below table:

| Risk | Liability | Motif |
|---|---|---|
| High | Glycosylation | NXS or NXT |
| | Free Cysteine | C |
| | Deamidation | NG, NS or QG |
| | Isomerization | DG, DP or DS |
| Medium | Deamidation | NH |
| | Hydrolysis | NP |
| | Cleavage | TS, KK, RK or KR |
| | Transglutaminase | LLQG (SEQ ID NO: 190) |
| | Integrin binding | RGD or KGD |
| | Plastic binding | FHENSP (SEQ ID NO: 191) |
| | | or WXXW |

At the start of the humanization process, a homology model of the parental VH and VL is built in a single chain Fv (scFv) format. Modeling is done in 4 stages: gathering homologous sequences; fold library scanning; loop modeling; side chain placement. The resulting model is used to guide the choice of “donor” or “acceptor” amino acids during the humanization process.

The parental VH and VL sequences are aligned with a panel of human germline sequences. This panel has been filtered to select germline sequences that do not contain unwanted sequence liabilities, particularly N-linked glycosylation sites and free cysteines. The closest matching germlines from two different VH and VL families are selected. A humanization algorithm is then used to select CDR and framework amino acids to graft from the donor parental sequences onto the human acceptor germline sequence. Four VH and four VL sequences are produced, giving a possible 16 antibodies. Percentage identity to human is calculated using the IMGT Domain Gap Align tool (Ehrenmann F., Kaas Q. and Lefranc M.-P. Nucleic Acids Res., 2010; 38:D301-D307). Sequence liabilities are determined based on sequence motifs.

The VH and VL sequences were run through the IGMT Gap Align tool to analyse against all known antibody germline sequences. CDR regions were assigned using the IMGT definition. As expected, the sequence is most closely aligned to mouse, specifically the IGHV1-4*01 family for the VH and IGK1-117*01 for the VL. The closest matching germlines for the parental SEQ ID NO: 1 were germlines IGHV1-46*01, IGHV7-4-1*02, IGKV2-30*01 and IGKV4-1*01.

The below table summarises the original parental and humanized sequences:

| | | Human | % identity |
|---|---|---|---|
| ID | Format | germline | to human |
| Parental | | — | 66.3% |
| hu-VH1 | Humanized | IGHV1-46*01 | 84.7% |
| hu-VH2 | Humanized | IGHV1-46*01 | 87.8% |
| hu-VH3 | Humanized | IGHV7-4-1*02 | 82.7% |
| hu-VH4 | Humanized | IGHV7-4-1*02 | 86.7% |
| Parental | | — | 80.0% |
| hu-VL1 | Humanized | IGKV2-30*01 | 90.0% |
| hu-VL2 | Humanized | IGKV2-30*01 | 91.0% |
| hu-VL3 | Humanized | IGKV4-1*01 | 80.2% |
| hu-VL4 | Humanized | IGKV4-1*01 | 81.2% |

The combination of 4 different heavy and light chain framework regions resulted in the generation of 16 different VH/VL combinations.

The different VH/VL combinations of the 16 antibodies are summarised in the below table:

| Proposed antibodies | | VH name | VL name |
|---|---|---|---|
| | Parental | VH | VL |
| 1 | Humanized-v1 | hu-VH1 | hu-VL1 |
| 2 | Humanized-v2 | hu-VH1 | hu-VL2 |
| 3 | Humanized-v3 | hu-VH1 | hu-VL3 |
| 4 | Humanized-v4 | hu-VH1 | hu-VL4 |
| 5 | Humanized-v5 | hu-VH2 | hu-VL1 |
| 6 | Humanized-v6 | hu-VH2 | hu-VL2 |
| 7 | Humanized-v7 | hu-VH2 | hu-VL3 |
| 8 | Humanized-v8 | hu-VH2 | hu-VL4 |
| 9 | Humanized-v9 | hu-VH3 | hu-VL1 |
| 10 | Humanized-v10 | hu-VH3 | hu-VL2 |
| 11 | Humanized-v11 | hu-VH3 | hu-VL3 |
| 12 | Humanized-v12 | hu-VH3 | hu-VL4 |
| 13 | Humanized-v13 | hu-VH4 | hu-VL1 |
| 14 | Humanized-v14 | hu-VH4 | hu-VL2 |
| 15 | Humanized-v15 | hu-VH4 | hu-VL3 |
| 16 | Humanized-v16 | hu-VH4 | hu-VL4 |

All humanized IgG1 formats expressed well and were successfully purified for characterization by SDS-PAGE and SEC-HPLC to monitor protein quality. The titre, amount of final antibody purified and the monomer content are described in the below table:

| | | | Titre | Purified | Monomer |
|---|---|---|---|---|---|
| ID | Format | % | (mg/l) | (mg) | (%) |
| Parental | IgG1 | VH: 66.3 | 10 | 0.8 | 100 |
| | | VL: 80 | | | |
| V1 | Humanized | VH: 84.7 | 22.5 | 1.8 | 99.2 |
| | human IgG1 | VL: 90.0 | | | |
| V2 | Humanized | VH: 84.7 | 1.25 | 0.1 | 100 |
| | human IgG1 | VL: 91.0 | | | |
| V3 | Humanized | VH: 84.7 | 50 | 4 | 100 |
| | human IgG1 | VL: 80.2 | | | |
| V4 | Humanized | VH: 84.7 | 50 | 4 | 99.4 |
| | human IgG1 | VL: 81.2 | | | |
| V5 | Humanized | VH: 87.8 | 24.5 | 1.4 | 98.0 |
| | human IgG1 | VL: 90.0 | | | |
| V6 | Humanized | VH: 87.8 | 8.5 | 0.7 | 97.8 |
| | human IgG1 | VL: 91.0 | | | |
| V7 | Humanized | VH: 87.8 | 47.5 | 3.8 | 98.5 |
| | human IgG1 | VL: 80.2 | | | |
| V8 | Humanized | VH: 87.8 | 40 | 3.2 | 98.5 |
| | human IgG1 | VL: 81.2 | | | |
| V9 | Humanized | VH: 82.7 | 15 | 1.2 | 99.14 |
| | human IgG1 | VL: 90.0 | | | |
| V10 | Humanized | VH: 82.7 | 10 | 0.8 | 100 |
| | human IgG1 | VL: 91.0 | | | |
| V11 | Humanized | VH: 82.7 | 52.5 | 4.2 | 99.5 |
| | human IgG1 | VL: 80.2 | | | |
| V12 | Humanized | VH: 82.7 | 49 | 3.9 | 99.7 |
| | human IgG1 | VL: 81.2 | | | |
| V13 | Humanized | VH: 86.7 | 2.5 | 0.2 | 100 |
| | human IgG1 | VL: 90.0 | | | |
| V14 | Humanized | VH: 86.7 | 2.5 | 0.2 | 99.4 |
| | human IgG1 | VL: 91.0 | | | |
| V15 | Humanized | VH: 86.7 | 27.5 | 2.2 | 99.6 |
| | human IgG1 | VL: 80.2 | | | |
| V16 | Humanized | VH: 86.7 | 37.5 | 3 | 100 |
| | human IgG1 | VL: 81.2 | | | |

### Example 3—Characterisation of Humanised Antibodies

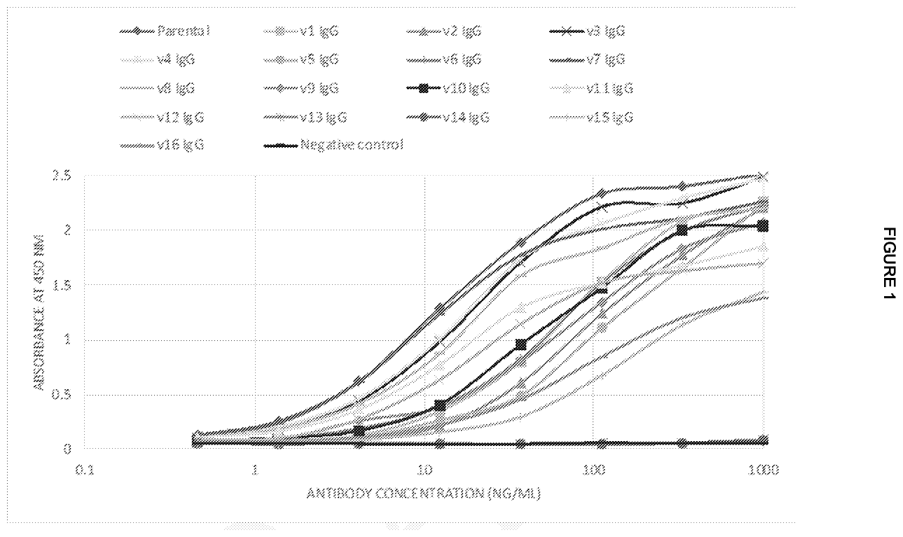

All 16 IgG format and scFv format humanised antibodies were assessed for their ability to binding to target (FLAG tag) using ELISA. Results are shown in FIG. 1.

Although most humanised variants retained the ability to bind to target, 3 humanised IgG clones (v3, v4 and v7) had greater affinity. These three IgG clones and corresponding scFV formats were selected for further analysis.

Humanized antibodies were able to demonstrate high affinity binding comparable to a parental mouse antibody (ie mouse VH/VL in the context of human constant regions).

The below table summarises the antibodies affinity and stoichiometry as measured by Biacore:

| Antibody | KD (M) | Binding Stoichiometry |
|---|---|---|
| V4 scFv | 2.2E−08 | 2.8 |
| V3 scFv | 1.9E−08 | 5.5 |
| V7 scFv | 1.5E−08 | 3.7 |
| V7 IgG | 1.3E−09 | 1.1 |
| V3 IgG | 1.0E−09 | 1.2 |
| Parental | 9.6E−10 | 1.3 |
| V4 IgG | 9.1E−10 | 1.1 |

Four IgG antibodies (parental mouse antibody and 3 humanised antibodies: v3, v4 and v7) were compared with the corresponding humanised scFvs to a single antigen binding by Biacore. Mouse scFv was not able to be expressed and therefore not able to be used for affinity comparison. IgG antibodies bound more tightly to the antigen in comparison to scFv by almost 10-fold. This is expected as IgG is bivalent in nature and the avidity will result in tighter binding as compared to monovalent expressed scFv.

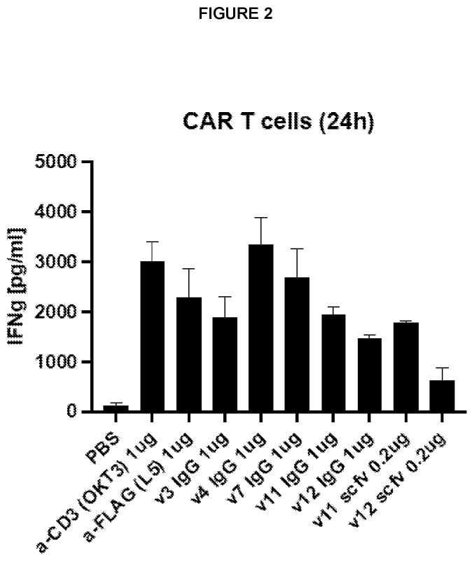

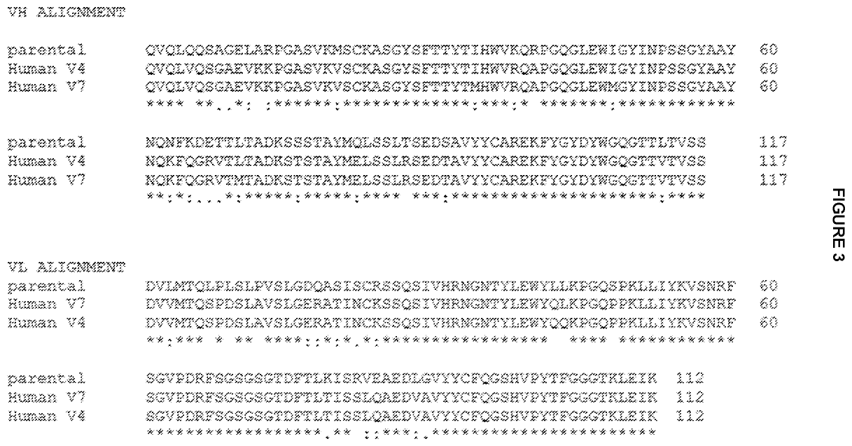

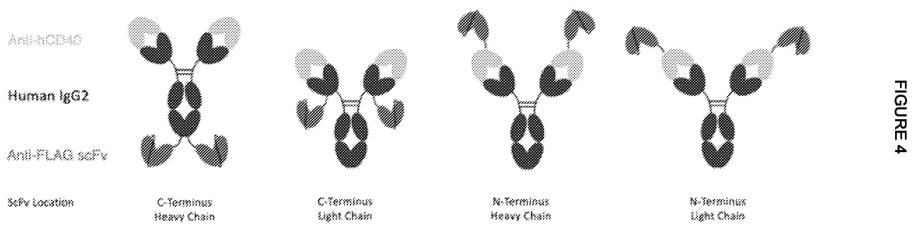

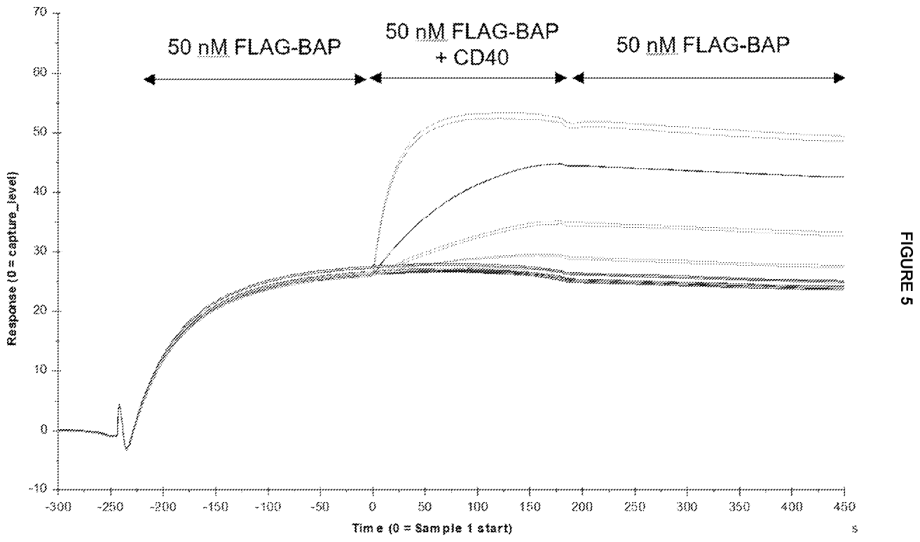

Antibodies v3, v4, v7 and v11 (in IgG format and also in scFv format in the case of v11) were further characterised. The antibodies were evaluated for their ability to activate anti-HER2-FLAG CAR T cells and induce IFN-γ. The antibodies induced IFN-γ expression as shown in FIG. 2. Antibody v11 was also included in the assessment in both IgG and scFv formats. The results indicate that antibodies v7 and v4, respectively, induced the greatest amounts of IFN-γ by CAR T cells.

Based on these results, antibody variants v4 and v7 scFv were selected for future studies.

### Example 4—anti-CD40-Anti-FLAG BEAT (BEAT1) Design and Affinity

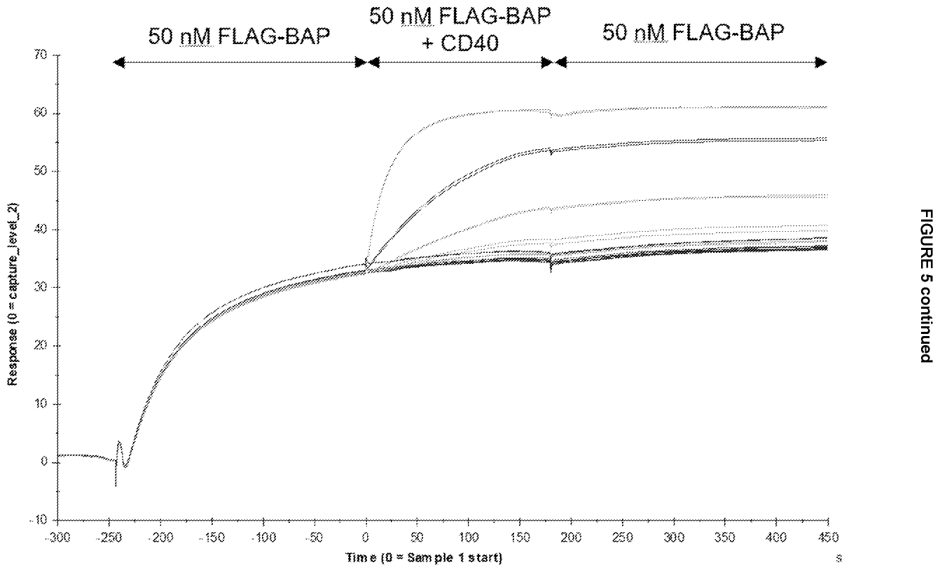

12 anti-CD40-anti-FLAG BEATs (collectively termed BEAT1) were designed and evaluated. The orientation of IgG and scFv domains are outline in FIG. 4. The 12 variations of BEAT1 s generated are summarised in the table below:

| Short | | | |
|---|---|---|---|
| name | Description | Heavy chain | Light chain |
| BEAT1a1 | Nter of Light chain with VL- | HC-IgG2(anti-CD40) | hM2V4_CurrusLinker- |
| | (GGGGS)3)-VH | | (GGGGS)3-LC- |
| | | | IgG2(anti-CD40) |
| BEAT1a2 | Cter of Light chain with VL- | HC-IgG2(anti-CD40) | LC-IgG2(anti-CD40) |
| | (GGGGS)3)-VH | | (GGGGS)3- |
| | | | hM2V4_CurrusLinker |
| BEAT1a3 | Nter of Heavy chain with VL- | hM2V4_CurrusLinker- | LC-IgG2(anti-CD40) |
| | (GGGGS)3)-VH | (GGGGS)3-HC- | |
| | | IgG2(anti-CD40) | |
| BEAT1a4 | Cter of Heavy chain with VL- | HC-IgG2(anti-CD40)- | LC-IgG2(anti-CD40) |
| | (GGGGS)3)-VH | (GGGGS)3- | |
| | | hM2V4_CurrusLinker | |
| BEAT1a5 | Nter of Light chain with VH- | HC-IgG2(anti-CD40) | hM2V4_PTGXLinker- |
| | PTGXlinker-VL | | (GGGGS)3-LC-IgG2(anti- |
| | | | CD40) |
| BEAT1a6 | Cter of Light chain with VH- | HC-IgG2(anti-CD40) | LC-IgG2(anti-CD40)- |
| | PTGXlinker-VL | | (GGGGS)3- |
| | | | hM2V4_PTGXLinker |
| BEAT1a7 | Nter of Heavy chain with VH- | hM2V4_PTGXLinker- | LC-IgG2(anti-CD40) |
| | PTGXlinker-VL | (GGGGS)3-HC- | |
| | | IgG2(anti-CD40) | |
| BEAT1a8 | Cter of Heavy chain with VH- | HC-IgG2(anti-CD40)- | LC-IgG2(anti-CD40) |
| | PTGXlinker-VL | (GGGGS)3- | |
| | | hM2V4_PTGXLinker | |
| BEAT1b1 | Nter of Light chain with VL- | HC-IgG2(anti-CD40) | hM2V7_CurrusLinker- |
| | (GGGGS)3)-VH | | (GGGGS)3-LC- |
| | | | IgG2(anti-CD40) |
| BEAT1b2 | Cter of Light chain with VL- | HC-IgG2(anti-CD40) | LC-IgG2(anti-CD40)- |
| | (GGGGS)3)-VH | | (GGGGS)3- |
| | | | hM2V7_CurrusLinker |
| BEAT1b3 | Nter of Heavy chain with VL- | hM2V7_CurrusLinker- | LC-IgG2(anti-CD40) |
| | (GGGGS)3)-VH | (GGGGS)3-HC- | |
| | | IgG2(anti-CD40) | |
| BEAT1b4 | Cter of Heavy chain with VL- | HC-IgG2(anti-CD40)- | LC-IgG2(anti-CD40) |
| | (GGGGS)3)-VH | (GGGGS)3- | |
| | | hM2V7_CurrusLinker | |

The linker used between VL-VH and also between the scFvs and IgG2 is (G4S)*3. The linker used between the VH-VL is 21 AAs

(GGGGSGGGGSGGGGSGGGGAS).

Binding of CD40 against 11 anti-CD40 BEAT antibodies, anti-CD40 IgG control and hCD40-cMyc cBEAT were characterized using multi-cycle SPR experiments. At least two reliable data sets were obtained for the binding of each anti-CD40 antibody to CD40. The binding of CD40 to all Anti-CD40 BEAT antibodies, produced good fits with high affinities, and K_{D}s in the 0.4-2 nM range. The binding of CD40 to the hCD40-cMyc rBEAT showed a K_{D} of 0.05-0.06 nM, while the control antibody anti-CD40 IgG2 showed a K_{D} of 0.5 nM (Table 3).

Binding of FLAG-BAP against anti-CD40 BEAT antibodies, and 3 anti-FLAG IgG control antibodies were characterized using multi-cycle SPR experiments. At least two reliable data sets were obtained for the binding of each BEAT binding to FLAG. The binding of FLAG-BAP produced good fits with high affinities, and K_{D}s in the 1-70 nM range. The binding of FLAG-BAP to control anti-FLAG antibodies showed a K_{D} ranging from 0.4-35 nM (Table 4).

TABLE 3
Affinity of BEAT1 to CD40 antigen

| | | kₐ | SE(kₐ) | | SE(k_{d}) | |
|---|---|---|---|---|---|---|
| Antigen | Antibody | (M⁻¹s⁻¹) | (M⁻¹s⁻¹) | k_{d} (s⁻¹) | (s⁻¹) | K_{D} (M) |
| CD40 | anti-CD40 IgG2 | 4.66E+05 | 8.22E+02 | 3.26E−04 | 1.37E−06 | 7.00E−10 |
| CD40 | anti-CD40 IgG2 | 3.69E+05 | 1.34E+02 | 1.97E−04 | 5.09E−07 | 5.35E−10 |
| CD40 | anti-CD40 IgG2 | 3.63E+05 | 2.39E+02 | 1.94E−04 | 5.79E−07 | 5.35E−10 |
| CD40 | BEAT 1a1 | 2.23E+05 | 3.56E+01 | 1.28E−04 | 2.40E−08 | 5.74E−10 |
| CD40 | BEAT 1a1 | 2.22E+05 | 1.06E+02 | 1.30E−04 | 5.27E−07 | 5.85E−10 |
| CD40 | BEAT 1a1 | 2.15E+05 | 1.06E+02 | 1.23E−04 | 5.29E−07 | 5.72E−10 |
| CD40 | BEAT 1a2 | 3.13E+05 | 1.47E+02 | 1.47E−04 | 6.22E−07 | 4.71E−10 |
| CD40 | BEAT 1a2 | 3.18E+05 | 1.77E+02 | 1.70E−04 | 7.42E−07 | 5.34E−10 |
| CD40 | BEAT 1a3 | 2.58E+05 | 1.33E+02 | 2.52E−04 | 6.21E−07 | 9.75E−10 |
| CD40 | BEAT 1a3 | 2.63E+05 | 1.71E+02 | 2.51E−04 | 7.93E−07 | 9.57E−10 |
| CD40 | BEAT 1a4 | 3.53E+05 | 1.24E+02 | 1.38E−04 | 4.86E−07 | 3.91E−10 |
| CD40 | BEAT 1a4 | 3.58E+05 | 1.55E+02 | 1.67E−04 | 6.03E−07 | 4.67E−10 |
| CD40 | BEAT 1a4 | 3.60E+05 | 1.39E+02 | 1.60E−04 | 5.38E−07 | 4.44E−10 |
| CD40 | BEAT 1a5 | 1.70E+05 | 9.66E+01 | 1.68E−04 | 5.12E−07 | 9.86E−10 |
| CD40 | BEAT 1a5 | 1.77E+05 | 6.46E+02 | 1.75E−04 | 9.96E−07 | 9.88E−10 |
| CD40 | BEAT 1a6 | 4.13E+05 | 1.60E+02 | 1.53E−04 | 5.56E−07 | 3.71E−10 |
| CD40 | BEAT 1a6 | 3.94E+05 | 9.56E+02 | 1.55E−04 | 6.48E−07 | 3.94E−10 |
| CD40 | BEAT 1a7 | 5.27E+05 | 5.81E+02 | 1.91E−04 | 1.61E−06 | 3.63E−10 |
| CD40 | BEAT 1a7 | 3.04E+05 | 1.70E+02 | 1.52E−04 | 7.30E−07 | 4.98E−10 |
| CD40 | BEAT 1a7 | 2.30E+05 | 3.00E+03 | 4.08E−04 | 2.74E−06 | 1.77E−09 |
| CD40 | BEAT 1a8 | 5.84E+05 | 5.73E+02 | 1.35E−04 | 1.43E−06 | 2.31E−10 |
| CD40 | BEAT 1a8 | 3.45E+05 | 2.01E+02 | 1.43E−04 | 7.20E−07 | 4.15E−10 |
| CD40 | BEAT 1a8 | 4.22E+05 | 9.85E+02 | 3.70E−04 | 1.67E−06 | 8.76E−10 |
| CD40 | BEAT 1b1 | 1.70E+05 | 1.27E+02 | 6.44E−05 | 6.73E−07 | 3.79E−10 |
| CD40 | BEAT 1b1 | 1.81E+05 | 9.34E+01 | 1.12E−04 | 4.89E−07 | 6.20E−10 |
| CD40 | BEAT 1b2 | 4.30E+05 | 1.52E+03 | 1.27E−04 | 9.66E−07 | 2.95E−10 |
| CD40 | BEAT 1b2 | 4.08E+05 | 9.74E+02 | 1.81E−04 | 6.68E−07 | 4.44E−10 |
| CD40 | BEAT 1b4 | 3.21E+05 | 1.08E+02 | 1.56E−04 | 4.51E−07 | 4.87E−10 |
| CD40 | BEAT 1b4 | 3.27E+05 | 1.21E+02 | 1.62E−04 | 5.01E−07 | 4.96E−10 |
| CD40 | hCD40-cMyc rBEAT | 7.94E+06 | 5.41E+03 | 3.82E−04 | 9.63E−07 | 4.82E−11 |
| CD40 | hCD40-cMyc rBEAT | 8.10E+06 | 6.99E+03 | 3.96E−04 | 1.22E−06 | 4.89E−11 |
| CD40 | hCD40-cMyc rBEAT | 7.02E+06 | 4.77E+03 | 4.00E−04 | 9.67E−07 | 5.70E−11 |

TABLE 4
Affinity of BEAT1 to FLAG tag

| | | kₐ | SE(kₐ) | | SE(k_{d}) | |
|---|---|---|---|---|---|---|
| Antigen | Antibody | (M⁻¹s⁻¹) | (M⁻¹s⁻¹) | k_{d} (s⁻¹) | (s⁻¹) | K_{D} (M) |
| FLAG-BAP | Anti-FLAG-L5 | 1.26E+06 | 6.91E+02 | 4.74E−04 | 7.72E−07 | 3.75E−10 |
| FLAG-BAP | Anti-FLAG-L5 | 1.24E+06 | 6.16E+02 | 4.55E−04 | 7.14E−07 | 3.68E−10 |
| FLAG-BAP | Anti-FLAG-M2-V4 | 6.72E+05 | 1.32E+03 | 1.39E−02 | 1.54E−05 | 2.07E−08 |
| FLAG-BAP | Anti-FLAG-M2-V4 | 4.46E+05 | 1.73E+03 | 1.53E−02 | 5.43E−05 | 3.43E−08 |
| FLAG-BAP | Anti-FLAG-M2-V7 | 6.66E+05 | 3.47E+03 | 1.84E−02 | 9.47E−05 | 2.77E−08 |
| FLAG-BAP | Anti-FLAG-M2-V7 | 4.57E+05 | 9.47E+02 | 1.06E−02 | 1.76E−05 | 2.32E−08 |
| FLAG-BAP | BEAT 1a1 | 2.97E+06 | 1.29E+04 | 1.99E−03 | 6.49E−06 | 6.71E−10 |
| FLAG-BAP | BEAT 1a1 | 2.36E+06 | 1.29E+04 | 1.89E−03 | 7.89E−06 | 7.98E−10 |
| FLAG-BAP | BEAT 1a2 | 2.48E+06 | 7.87E+03 | 3.12E−03 | 4.09E−06 | 1.26E−09 |
| FLAG-BAP | BEAT 1a2 | 1.69E+06 | 5.11E+03 | 3.37E−03 | 5.50E−06 | 1.99E−09 |
| FLAG-BAP | BEAT 1a3 | 2.43E+06 | 8.91E+03 | 2.96E−03 | 4.59E−06 | 1.22E−09 |
| FLAG-BAP | BEAT 1a3 | 1.84E+06 | 2.97E+04 | 3.03E−03 | 4.06E−05 | 1.65E−09 |
| FLAG-BAP | BEAT 1a3 | 3.12E+06 | 8.84E+03 | 3.87E−03 | 5.36E−06 | 1.24E−09 |
| FLAG-BAP | BEAT 1a4 | 3.29E+05 | 4.84E+02 | 2.27E−03 | 2.75E−06 | 6.90E−09 |
| FLAG-BAP | BEAT 1a4 | 4.56E+05 | 9.14E+02 | 2.30E−03 | 3.85E−06 | 5.04E−09 |
| FLAG-BAP | BEAT 1a5 | 1.59E+06 | 1.14E+04 | 2.45E−02 | 1.59E−04 | 1.54E−08 |
| FLAG-BAP | BEAT 1a5 | 1.64E+06 | 5.95E+03 | 3.58E−02 | 1.05E−04 | 2.19E−08 |
| FLAG-BAP | BEAT 1a6 | 6.20E+05 | 1.47E+03 | 1.70E−02 | 3.51E−05 | 2.74E−08 |
| FLAG-BAP | BEAT 1a6 | 8.80E+05 | 3.32E+03 | 1.37E−02 | 5.16E−05 | 1.55E−08 |
| FLAG-BAP | BEAT 1a7 | 2.97E+06 | 2.22E+04 | 3.23E−02 | 2.61E−04 | 1.09E−08 |
| FLAG-BAP | BEAT 1a7 | 2.65E+06 | 1.00E+04 | 2.07E−02 | 8.42E−05 | 7.81E−09 |
| FLAG-BAP | BEAT 1a7 | 9.26E+05 | 1.71E+03 | 1.25E−02 | 1.47E−05 | 1.35E−08 |
| FLAG-BAP | BEAT 1a8 | 4.57E+05 | 5.58E+02 | 4.62E−03 | 5.65E−06 | 1.01E−08 |
| FLAG-BAP | BEAT 1a8 | 4.67E+05 | 5.18E+02 | 4.13E−03 | 4.00E−06 | 8.85E−09 |
| FLAG-BAP | BEAT 1b1 | 2.40E+06 | 1.06E+04 | 2.86E−03 | 6.67E−06 | 1.19E−09 |
| FLAG-BAP | BEAT 1b1 | 2.31E+06 | 1.12E+04 | 4.21E−03 | 1.44E−05 | 1.82E−09 |
| FLAG-BAP | BEAT 1b1 | 3.39E+06 | 1.70E+04 | 3.93E−03 | 1.55E−05 | 1.16E−09 |
| FLAG-BAP | BEAT 1b2 | 9.06E+05 | 6.85E+03 | 5.87E−03 | 4.16E−05 | 6.48E−09 |
| FLAG-BAP | BEAT 1b2 | 7.72E+05 | 2.66E+03 | 6.52E−03 | 1.61E−05 | 8.44E−09 |
| FLAG-BAP | BEAT 1b4 | 5.92E+05 | 3.37E+03 | 2.72E−03 | 1.44E−05 | 4.61E−09 |
| FLAG-BAP | BEAT 1b4 | 5.39E+05 | 9.16E+02 | 4.24E−03 | 7.19E−06 | 7.86E−09 |

Next, BEAT 1a4 (F7-G8) simultaneous CD40 and FLAG-BAP binding experiments were conducted. In these experiments BEAT 1a4 was captured on the Protein G surface, then 50 nM FLAG-BAP was injected until saturation was achieved, the CD40 was then immediately injected in the presence of 50 nM FLAG-BAP, then dissociation of CD40 was monitored while only 50 nM FLAG-BAP was injected. This was performed for different concentrations of CD40 which allowed the measurement of association and dissociation of CD40 with BEAT 1a4, which was constantly saturated with FLAG-BAP. Similarly, the reverse experiment was carried out, where after capture of BEAT 1a4, 50 nM CD40 was injected until saturation, followed by FLAG-BAP in the presence of 50 nM CD40, then FLAG-BAP dissociation was monitored while injecting 50 nM CD40 only. These experiments showed that BEAT 1a4 was able to bind CD40 while saturated with FLAG-BAP (FIG. 5). Under these conditions CD40 bound with similar kinetic parameters, affinity and Rmax in the presence or absence of bound FLAG-BAP. Likewise, FLAG-BAP binds to BEAT1a4 while saturated with bound CD40. FLAG-BAP binding to BEAT 1a4 also shows similar kinetic parameters, affinity and Rmax in the presence or absence of bound CD40.

### Example 5—Binding of BEAT1 to APCs

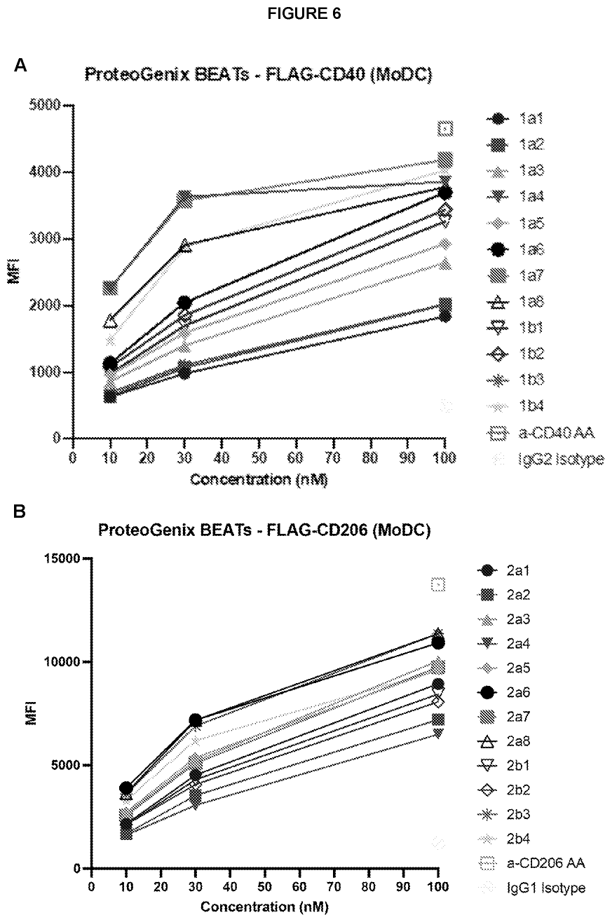

BEAT1s were evaluated for binding to APCs at 10, 30 and 100 nM. The IgG2 isotype control had no binding and the anti-CD40 antibody control had the highest binding (100 nM only) (FIG. 6A). BEATs 1a7, 1a4 and 1b4 had the highest binding.

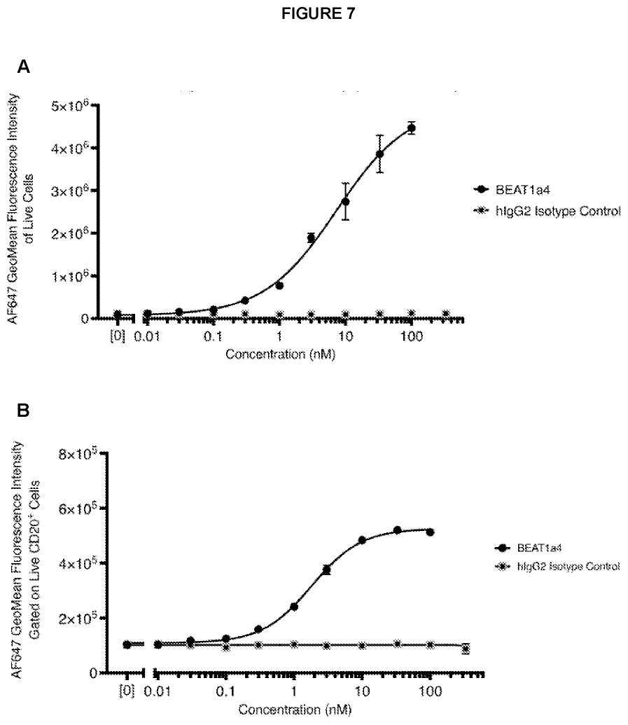

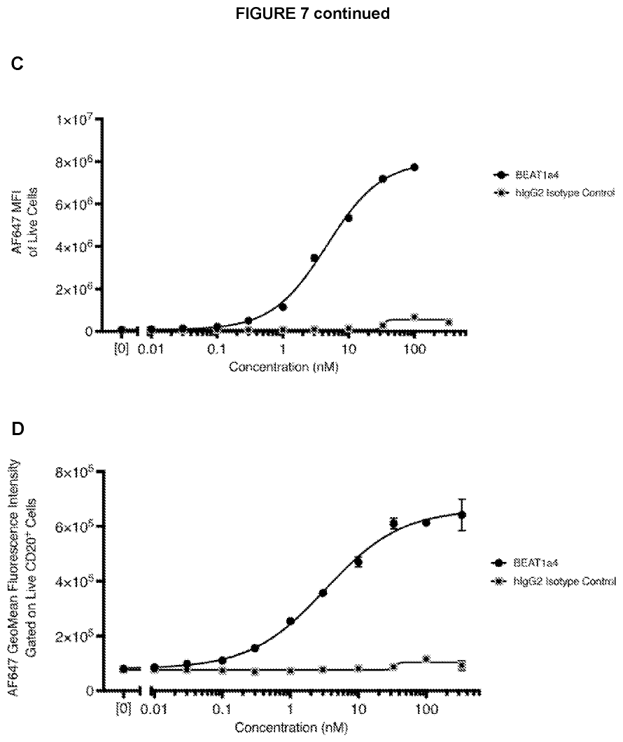

BEAT1a4 demonstrated dose-dependent binding to CD40 on human MoDC cells between 0.1-100 nM (FIG. 7A) and human B Cells (FIG. 7B) and to CD40 on non-human primate MoDC cells between 0.1-100 nM (FIG. 7C) and non-human primate B Cells (FIG. 7D).

Binding of BEAT 1a4 to human cells/DCs: cell binding using two (2) test articles (antibody biologics), including one (1) test article of interest (Bispecific antibody 1 (BEAT 1a4), and one (1) human IgG2 isotype control antibody. PBMCs and MoDCs from 3 healthy human donors were evaluated. Binding of test articles to CD3+, CD20+, CD14+, and CD11c+ cell subsets was evaluated. A dose response of each test article of interest and isotype control antibodies with a 200 nM top concentration followed by a two-fold dilution series (10 points total) and an untreated sample, followed by a secondary antibody (5 μg/mL), was examined in duplicate for binding on all proposed cell types (FIGS. 7A and 7B).

Binding of BEAT 1a4 to NHP cells/DCs: cell binding using two (2) test articles (antibody biologics), including one (1) test article of interest (Bispecific antibody 1 (BEAT 1a4), and one (1) human IgG2 isotype control antibody. PBMCs and MoDCs from 3 NHP donors was evaluated. Binding of test articles to CD3+, CD20+, CD14+, and CD11c+ cell subsets were evaluated. A dose response of each test article of interest and isotype control antibodies with a 200 nM top concentration followed by a two-fold dilution series (10 points total) and an untreated sample, followed by a secondary antibody (5 μg/mL), was examined in duplicate for binding on all proposed cell types (FIGS. 7C and 7D).

### Example 6—Binding of BEAT1 to CAR T Cells

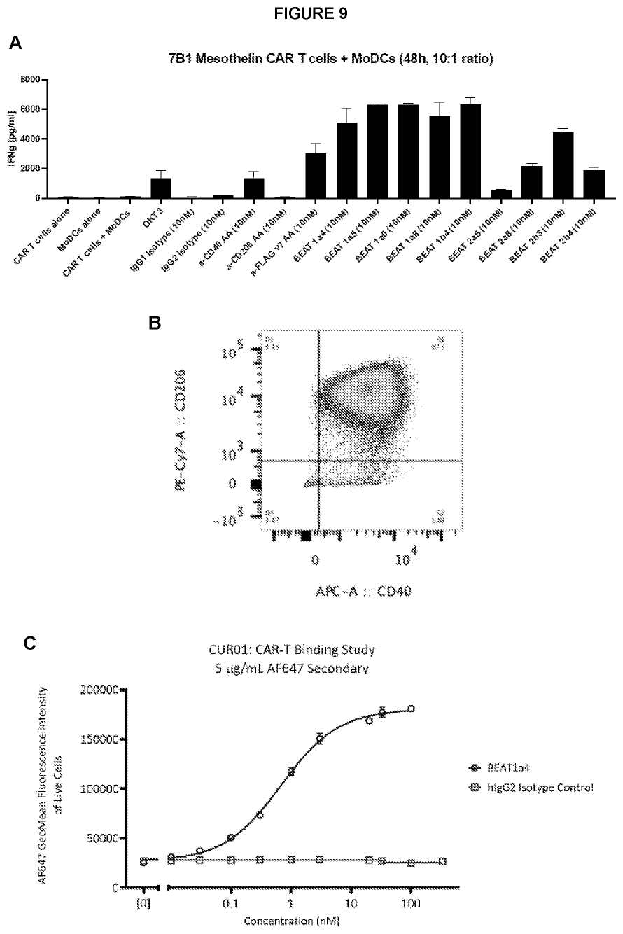

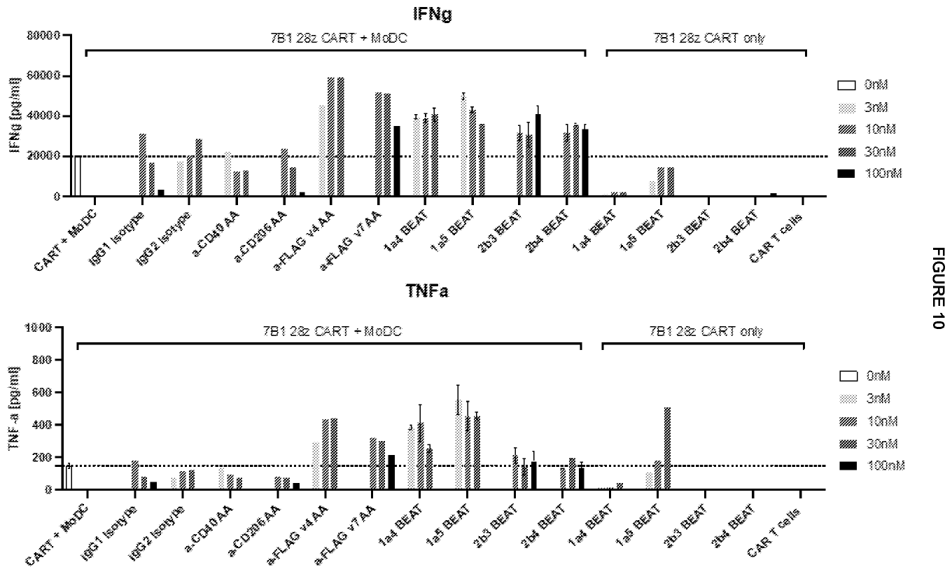

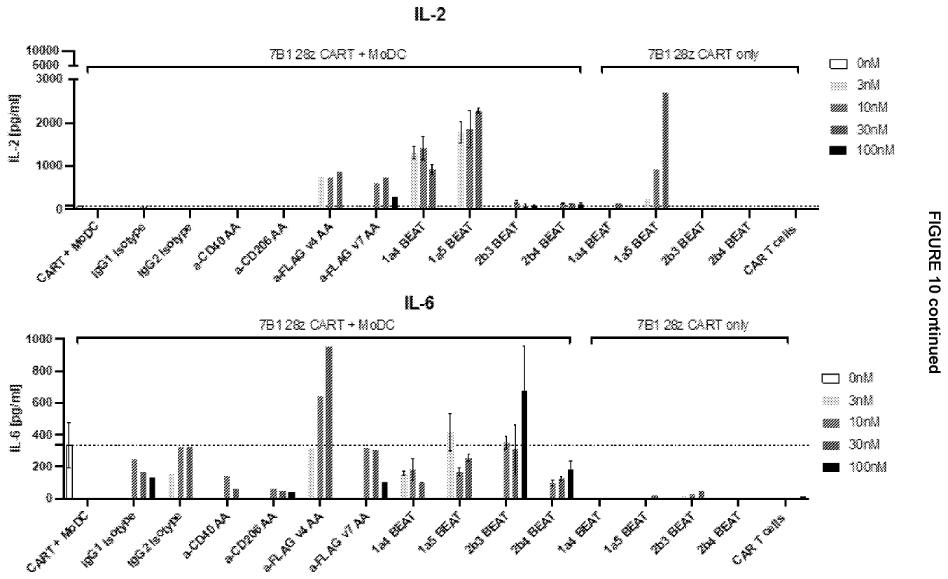

BEAT1a4 was evaluated for binding to CAR T-cells that had a FLAG tag and expressed GFP. Cells were detected by GFP+/AF647+ cells via FACs. BEAT1a4 binding was evaluated across a dose range of 0.1 nM-100 nM (FIG. 9C).

Binding of test article BEAT1a4 to CAR-T target cells that have a FLAG tag was evaluated in a 10 point standard curve to confirm the EC50. After viability dye staining and incubation with 5% FBS to block non-specific binding, CAR-T cells were incubated with a dose response of either BEAT1a4 or human IgG2 isotype control. The following concentrations were tested, in duplicate: 0.01, 0.03, 0.1, 0.3, 1, 3, 10, 33 and 100 nM, in addition to untreated controls. Following incubation, cells were washed and stained with a fluorescently-labelled secondary reagent (AF647—conjugated AffiniPure Fab2 fragment Goat anti-human IgG Fcg specific) at 5 μg/mL, then washed and acquired via high throughput flow cytometry. The GeoMean fluorescence intensity (MFI) values of AF647 within the live cells (viability dye-negative) were plotted on an XY chart, graphing MFI against the log of the concentration and the data fit to a non-linear regression curve from which the EC50 was calculated. The percent AF647 cells gated on live cells was also analyzed.

The BEAT 1a4 demonstrated dose dependent binding to CAR T cells with an EC50 of 0.51 nM.

### Example 7—Functional Activity of BEAT1 on APCs and CAR T Cells

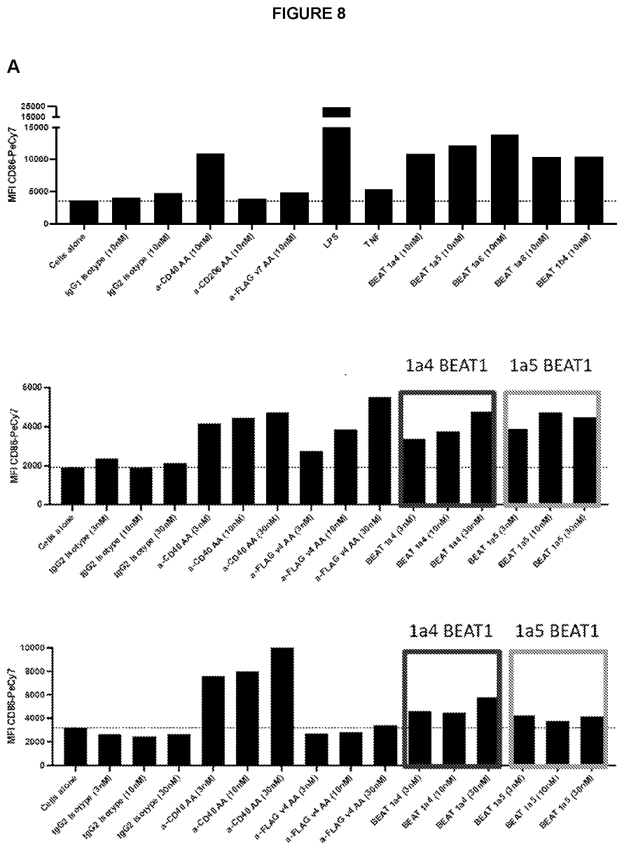

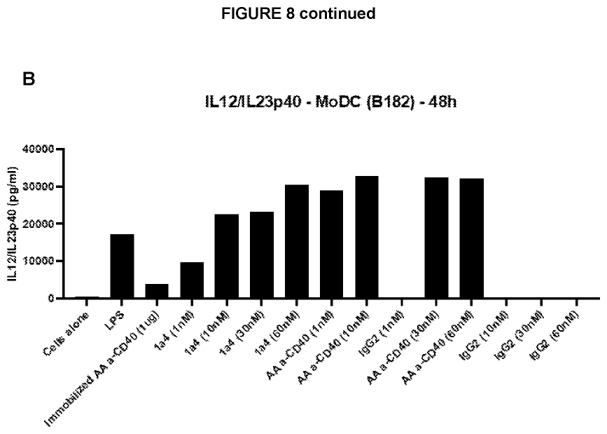

CD86 upregulation was evaluated for lead BEAT candidates with the addition of the BEATs to monocyte derived DCs (MoDCs). Agonism of CD40 is expected to upregulate CD86 expression. At 10 nM of BEAT 1a4, CD86 was upregulated in a dose-dependent manner with the addition of anti-CD40 BEATs BEAT1a4 and BEAT 1a5 (FIG. 8A). Control antibodies had no effect on CD86 increases.

BEAT1a4 (and an anti-CD40 agonist control) increased IL-12/IL-23p40 release in a dose responsive manner between 3-30 nM (CD86) and 1-60 nM (IL-12/IL-23p40) (FIG. 8B).

CAR T cells that included the FLAG tag, plus MoDCs were evaluated for functional activity with the addition of the BEAT1a4. MoDCs were confirmed to have CD40 expression (FIG. 9B). Increases in IFN-γ were seen with the addition of the anti-CD40 BEATs (FIG. 9A). Negative controls (IgG1 and IgG2 isotypes) had no effect on IFN-γ.

### Example 8—BEAT1 Cytokine Analysis

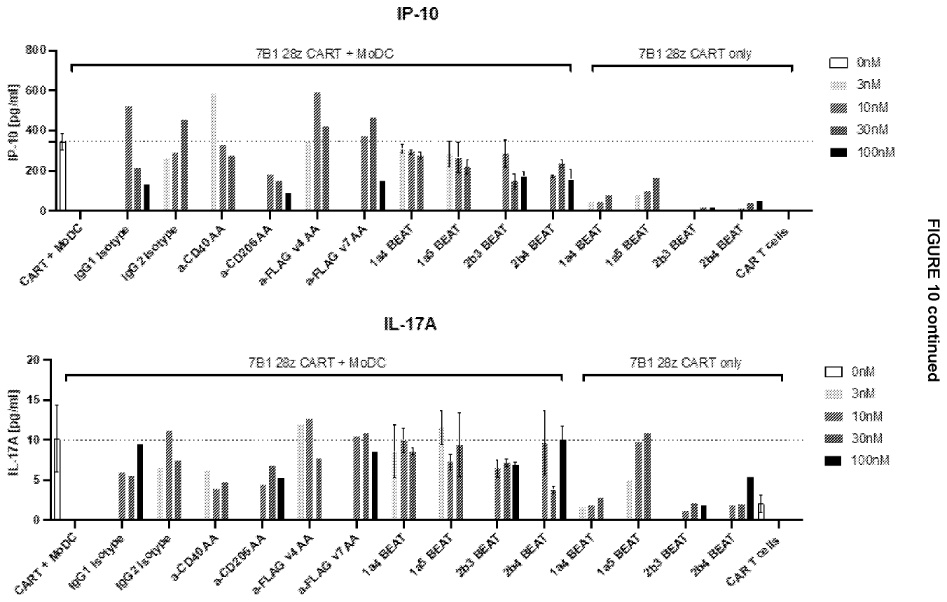

A cytokine analysis was completed with one donor to test BEAT1a4. anti-CD40 BEAT1 s stimulated IFN-γ, TNF-α, and IL2 with some activity with 1a4 and 1a5 at low doses with IL12-p70 (FIG. 10). There was limited activity with IL-6, IL-4, IL-10, IL-8, IP-10, MCP-1 and IL-17A above the cells alone background for BEAT1a4.

### Example 9—T-Cell Proliferation with Addition of BEAT1

The proliferation of 7B1 28z CAR T cells in the presence of control compounds/BEAT1 (30 nM) and MoDCs was examined after 120 hrs proliferation.

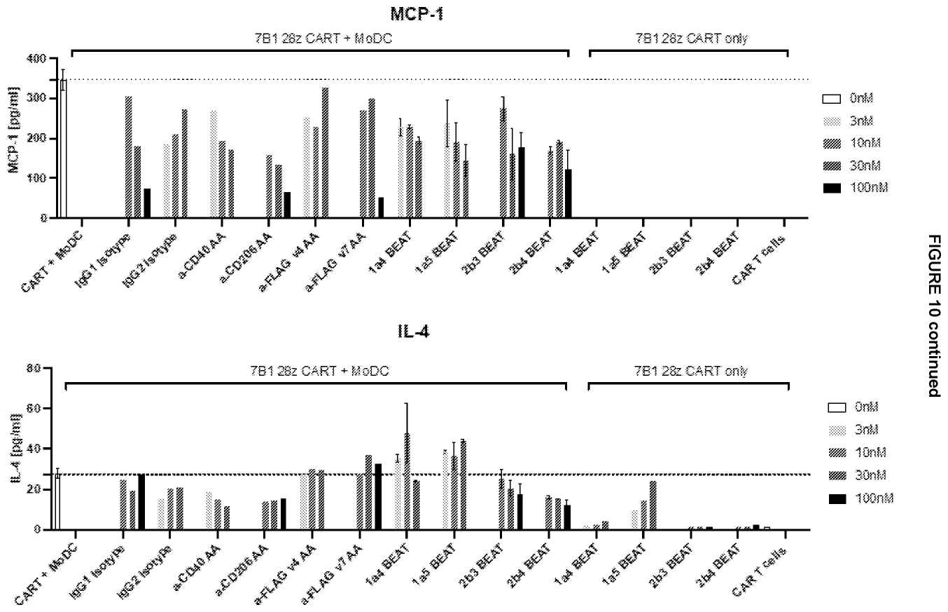

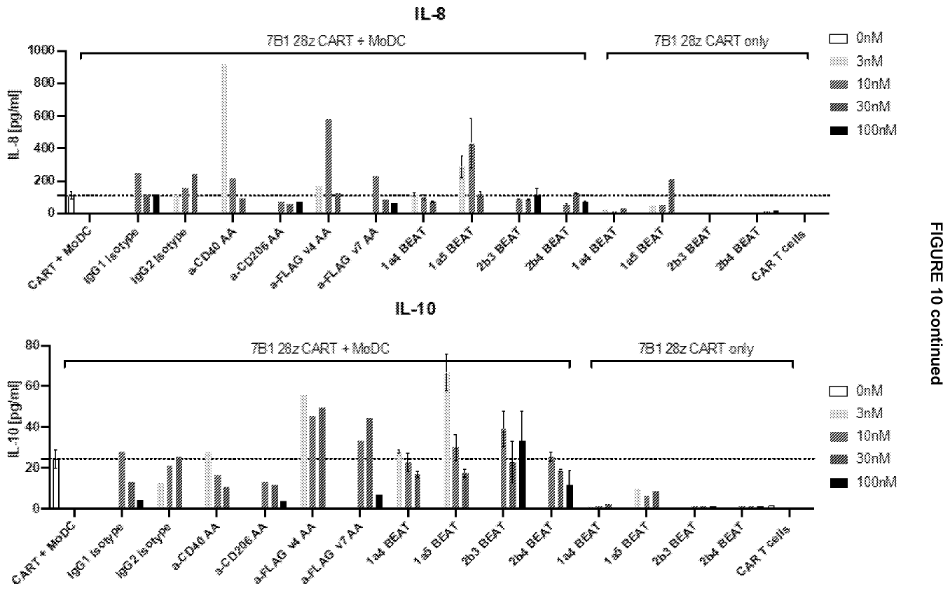

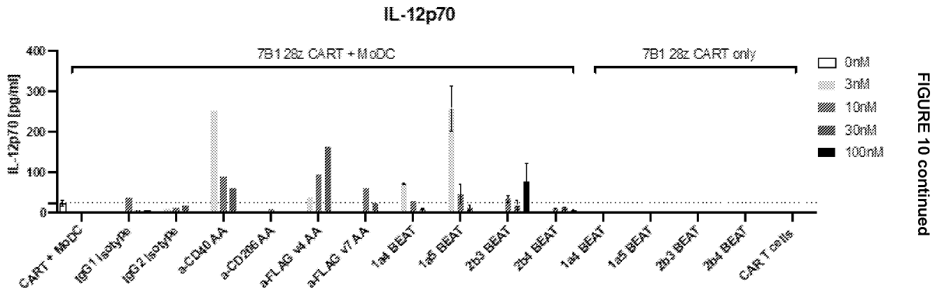

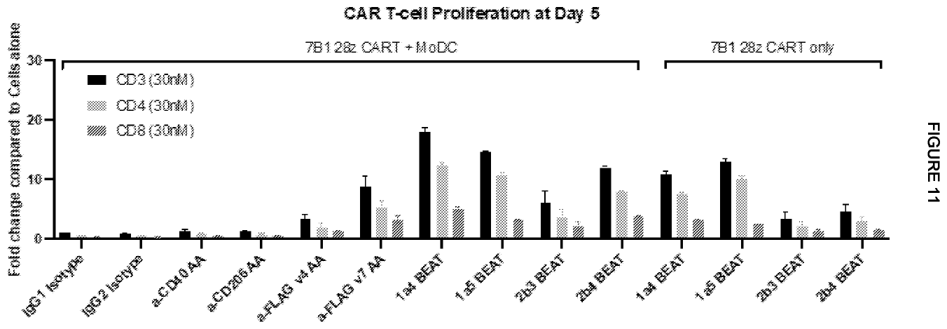

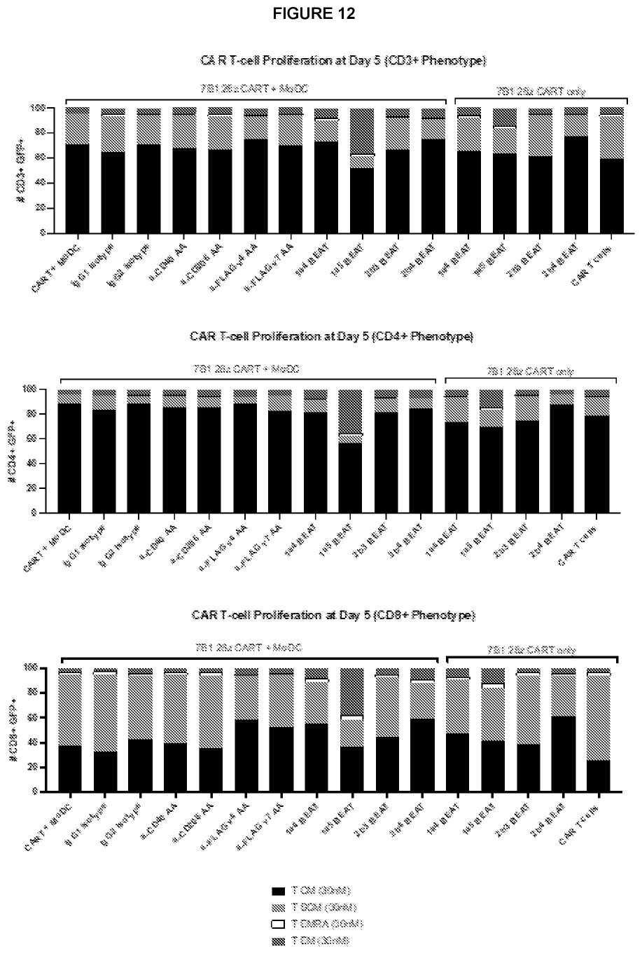

Significant increase in fold change was observed relative to controls for CD3+, CD4+ and CD8+ cells for anti-CD40 BEATs 1a4 and 1a5 (FIG. 11), in particular there was an increase the percentage of effector memory T cells with addition of the anti-CD40 BEAT1a4, in particular the CD8+ T cells (FIG. 12).

### Example 10—Cytotoxicity of BEAT1+CAR T

Cytotoxicity was measured by a luciferase assay using the ovarian cancer cell line SKOV-3 (parental, MSLN −ve and MSLN-expressing)+MoDC+CAR T.

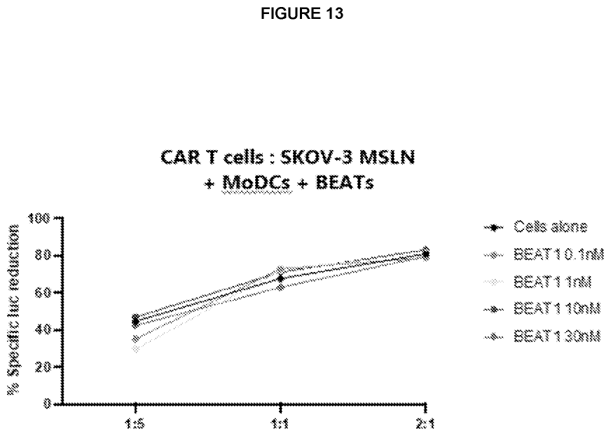

BEATs had no effect in MSLN-expressing SKOV-3 cells (FIG. 13).

### Example 11—In Vivo Efficacy of CAR T+BEAT

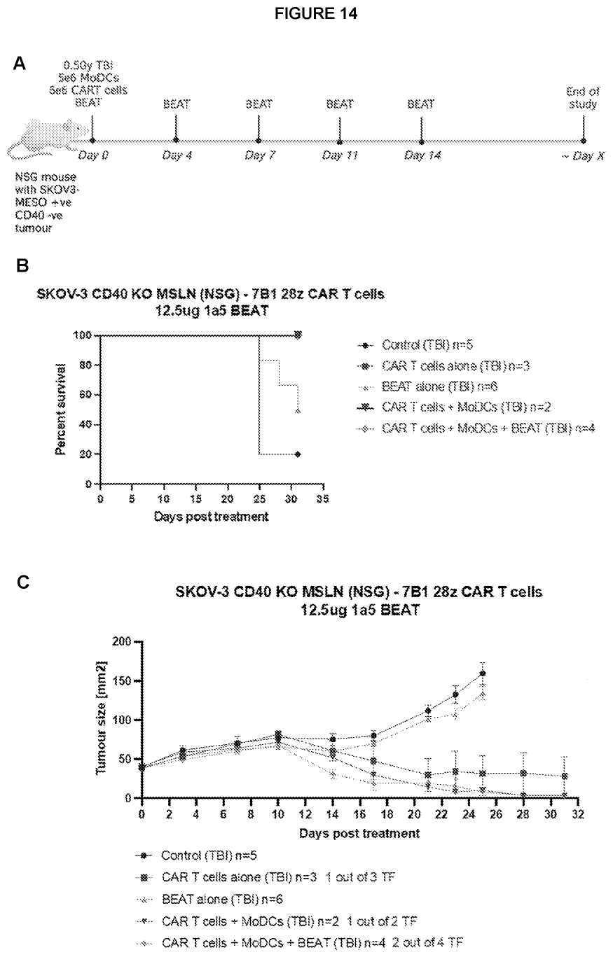

BEAT1a5 was evaluated in an NSG mouse model using SKOV3-Mesothelin transfected CD40 knock-out cells. Tumours were first established (50 mm²) in the NSG mice. A single dose of 5 million anti-mesothelin CARs/5 million MoDCs (with high expression of mesothelin) and bi-weekly doses of BEAT at 12.5 ug BEAT (5 doses) (FIG. 14A).

Survival and tumour size were measured (FIGS. 14B and C), with 2/4 mice tumour free at day 21 with CART cells+MoDCs+BEAT compared to ⅓ with CAR T cells alone.

### Example 12—Anti-CD206 Antibody Generation

An anti-CD206 antibody was obtained. 500 mL HEK293 cultures were transfected with anti-CD206 (B11) encoding vectors. Cultures were harvested by centrifugation at 6 days. An SDS-PAGE gel was completed at 3 days. Clarified supernatants of the IgG1 protein were purified by Protein A Affinity Chromatography ProtA (Agarose Resin) & Buffer exchanged (PBS). Materials were dialysed into PBS buffer. 15 μg samples were formulated in 4× loading buffer (reducing and non-reducing) and analyzed by SDS-PAGE. Based on sizes, the reducing conditions give >95% purity of the B11.

### Example 13—Anti-CD206-anti-FLAG BEAT2 Design and Characteristics

The anti-FLAGs were designed as described in Example 2 and 3 above. 12 anti-CD206-anti-FLAG BEATs (BEAT2) were designed and evaluated using the IgG and scFv domains as outlined in FIG. 4. The 12 variations of BEAT2s generated are summarised in the table below:

| Short | | | |
|---|---|---|---|
| name | Description | Heavy chain | Light chain |
| BEAT2a1 | Nter of Light chain with VL- | HC-IgG1LALA(anti-CD206) | hM2V4_CurrusLinker- |
| | (GGGGS)3)-VH | | (GGGGS)3-LC- |
| | | | IgG1LALA(anti-CD206) |
| BEAT2a2 | Cter of Light chain with VL- | HC-IgG1LALA(anti-CD206) | LC-IgG1LALA(anti-CD206)- |
| | (GGGGS)3)-VH | | (GGGGS)3- |
| | | | hM2V4_CurrusLinker |
| BEAT2a3 | Nter of Heavy chain with VL- | hM2V4_CurrusLinker- | LC-IgG1LALA(anti-CD206) |
| | (GGGGS)3)-VH | (GGGGS)3-HC- | |
| | | IgG1LALA(anti-CD206) | |
| BEAT2a4 | Cter of Heavy chain with VL- | HC-IgG1LALA(anti-CD206)- | LC-IgG1LALA(anti-CD206) |
| | (GGGGS)3)-VH | (GGGGS)3- | |
| | | hM2V4_CurrusLinker | |
| BEAT2a5 | Nter of Light chain with VH- | HC-IgG1LALA(anti-CD206) | hM2V4_PTGXLinker-(GGGGS)3- |
| | PTGXlinker-VL | | LC- |
| | | | IgG1LALA(anti-CD206) |
| BEAT2a6 | Cter of Light chain with VH- | HC-IgG1LALA(anti-CD206) | LC-IgG1LALA(anti-CD206)- |
| | PTGXlinker-VL | | (GGGGS)3- |
| | | | hM2V4_PTGXLinker |
| BEAT2a7 | Nter of Heavy chain with VH- | hM2V4_PTGXLinker- | LC-IgG1LALA(anti-CD206) |
| | PTGXlinker-VL | (GGGGS)3-HC- | |
| | | IgG1LALA(anti-CD206) | |
| BEAT2a8 | Cter of Heavy chain with VH- | HC-IgG1LALA(anti-CD206)- | LC-IgG1LALA(anti-CD206) |
| | PTGXlinker-VL | (GGGGS)3- | |
| | | hM2V4_PTGXLinker | |
| BEAT2b1 | Nter of Light chain with VL- | HC-IgG1LALA(anti-CD206) | hM2V7_Curruslinker- |
| | (GGGGS)3)-VH | | (GGGGS)3-LC- |
| | | | IgG1LALA(anti-CD206) |
| BEAT2b2 | Cter of Light chain with VL- | HC-IgG1LALA(anti-CD206) | LC-IgG1LALA(anti-CD206)- |
| | (GGGGS)3)-VH | | (GGGGS)3- |
| | | | hM2V7_Curruslinker |
| BEAT2b3 | Nter of Heavy chain with VL- | hM2V7_CurrusLinker- | LC-IgG1LALA(anti-CD206) |
| | (GGGGS)3)-VH | (GGGGS)3-HC- | |
| | | IgG1LALA(anti-CD206) | |
| BEAT2b4 | Cter of Heavy chain with VL- | HC-IgG1LALA(anti-CD206)- | LC-IgG1LALA(anti-CD206) |
| | (GGGGS)3)-VH | (GGGGS)3- | |
| | | hM2V7_CurrusLinker | |

The linker used between VL-VH and also between the scFvs and IgG2 is (G4S)*3. The linker used between the VH-VL is 21 AAs

(GGGGSGGGGSGGGGSGGGGAS).

Binding of CD206 against 12 anti-CD206 BEAT antibodies and anti-CD206 IgG control were characterized using multi-cycle SPR experiments. At least two reliable data sets were obtained for the binding of each anti-CD206 antibody to CD206. The binding of CD206 to all Anti-CD206 BEAT antibodies, produced good fits with high affinities, and K_{D}s in the 10-80 nM range. The binding of CD206 to the control antibody anti-CD206 IgG1 showed a KD of 11 nM. The association rate constant for all anti-CD206 BEAT antibodies against CD206 were similar (6×10³-3×10⁴ M⁻¹s⁻¹) as were the dissociation rate constants (2-5×10⁻⁴ s⁻¹). (Table 5).

Binding of FLAG-BAP against 12 BEAT antibodies, and 3 anti-FLAG IgG control antibodies were characterized using multi-cycle SPR experiments. At least two reliable data sets were obtained for the binding of each BEAT binding to FLAG with the exception of BEAT 2a1. The binding of FLAG-BAP to the BEAT antibodies, produced good fits with high affinities, and KDs in the 1-70 nM range. The binding of FLAG-BAP to control anti-FLAG antibodies showed a KD ranging from 0.4-35 nM. The association rate constant for all BEAT antibodies against FLAG-BAP ranged from 1×10⁵-5×10⁶ M⁻¹s⁻¹, while the dissociation rate constants ranged from 1×10⁻³-4×10⁻² s⁻¹. (Table 6).

TABLE 5
Affinity of BEAT2 to CD206 antigen

| | | kₐ | SE(kₐ) | | SE(k_{d}) | |
|---|---|---|---|---|---|---|
| Antigen | Antibody | (M⁻¹s⁻¹) | (M⁻¹s⁻¹) | k_{d} (s⁻¹) | (s⁻¹) | K_{D} (M) |
| CD206 | anti-CD206 IgG1 | 2.65E+04 | 1.05E+01 | 2.77E−04 | 3.99E−07 | 1.05E−08 |
| CD206 | anti-CD206 IgG1 | 2.31E+04 | 3.04E+01 | 2.58E−04 | 4.87E−07 | 1.12E−08 |
| CD206 | anti-CD206 IgG1 | 2.43E+04 | 9.75E+00 | 2.51E−04 | 5.67E−07 | 1.03E−08 |
| CD206 | anti-CD206 IgG1 | 2.30E+04 | 3.37E+01 | 2.55E−04 | 6.26E−07 | 1.11E−08 |
| CD206 | BEAT 2a1 | 1.48E+04 | 3.39E+01 | 2.85E−04 | 1.54E−06 | 1.92E−08 |
| CD206 | BEAT 2a1 | 6.15E+03 | 1.30E+01 | 4.73E−04 | 1.35E−06 | 7.68E−08 |
| CD206 | BEAT 2a2 | 2.45E+04 | 1.01E+01 | 2.32E−04 | 6.02E−07 | 9.48E−09 |
| CD206 | BEAT 2a2 | 2.27E+04 | 8.60E+00 | 2.68E−04 | 5.38E−07 | 1.18E−08 |
| CD206 | BEAT 2a3 | 1.51E+04 | 1.02E+01 | 3.56E−04 | 5.14E−07 | 2.36E−08 |
| CD206 | BEAT 2a3 | 1.46E+04 | 3.88E+01 | 3.24E−04 | 7.08E−07 | 2.22E−08 |
| CD206 | BEAT 2a4 | 2.41E+04 | 1.27E+01 | 2.87E−04 | 2.04E−07 | 1.19E−08 |
| CD206 | BEAT 2a4 | 2.12E+04 | 9.04E+00 | 2.19E−04 | 3.65E−07 | 1.03E−08 |
| CD206 | BEAT 2a5 | 1.20E+04 | 5.47E+00 | 3.61E−04 | 5.23E−07 | 3.01E−08 |
| CD206 | BEAT 2a5 | 1.20E+04 | 5.37E+00 | 3.93E−04 | 3.78E−07 | 3.27E−08 |
| CD206 | BEAT 2a6 | 2.46E+04 | 1.25E+01 | 2.54E−04 | 4.35E−07 | 1.03E−08 |
| CD206 | BEAT 2a6 | 2.47E+04 | 7.41E+00 | 2.56E−04 | 3.65E−07 | 1.04E−08 |
| CD206 | BEAT 2a7 | 1.92E+04 | 8.75E+00 | 3.05E−04 | 5.77E−07 | 1.59E−08 |
| CD206 | BEAT 2a7 | 1.76E+04 | 1.76E+01 | 2.51E−04 | 1.22E−06 | 1.43E−08 |
| CD206 | BEAT 2a8 | 2.20E+04 | 1.84E+01 | 3.43E−04 | 6.93E−07 | 1.56E−08 |
| CD206 | BEAT 2a8 | 2.10E+04 | 3.02E+01 | 3.32E−04 | 4.96E−07 | 1.58E−08 |
| CD206 | BEAT 2b1 | 9.66E+03 | 1.44E+01 | 2.68E−04 | 1.35E−06 | 2.78E−08 |
| CD206 | BEAT 2b1 | 8.16E+03 | 1.40E+01 | 3.84E−04 | 9.44E−07 | 4.70E−08 |
| CD206 | BEAT 2b2 | 2.26E+04 | 1.04E+01 | 2.86E−04 | 4.57E−07 | 1.27E−08 |
| CD206 | BEAT 2b2 | 2.36E+04 | 1.46E+01 | 3.17E−04 | 4.59E−07 | 1.34E−08 |
| CD206 | BEAT 2b3 | 1.63E+04 | 9.73E+00 | 3.28E−04 | 5.22E−07 | 2.01E−08 |
| CD206 | BEAT 2b3 | 1.60E+04 | 6.23E+00 | 3.42E−04 | 4.80E−07 | 2.13E−08 |
| CD206 | BEAT 2b4 | 2.25E+04 | 1.69E+01 | 2.79E−04 | 3.94E−07 | 1.24E−08 |
| CD206 | BEAT 2b4 | 2.13E+04 | 9.12E+00 | 2.98E−04 | 5.23E−07 | 1.40E−08 |

TABLE 6
Affinity of BEAT2 to FLAG tag

| | | kₐ | SE(kₐ) | | SE(k_{d}) | |
|---|---|---|---|---|---|---|
| Antigen | Antibody | (M⁻¹s⁻¹) | (M⁻¹s⁻¹) | k_{d} (s⁻¹) | (s⁻¹) | K_{D} (M) |
| FLAG-BAP | Anti-FLAG-L5 | 1.26E+06 | 6.91E+02 | 4.74E−04 | 7.72E−07 | 3.75E−10 |
| FLAG-BAP | Anti-FLAG-L5 | 1.24E+06 | 6.16E+02 | 4.55E−04 | 7.14E−07 | 3.68E−10 |
| FLAG-BAP | Anti-FLAG-M2-V4 | 6.72E+05 | 1.32E+03 | 1.39E−02 | 1.54E−05 | 2.07E−08 |
| FLAG-BAP | Anti-FLAG-M2-V4 | 4.46E+05 | 1.73E+03 | 1.53E−02 | 5.43E−05 | 3.43E−08 |
| FLAG-BAP | Anti-FLAG-M2-V7 | 6.66E+05 | 3.47E+03 | 1.84E−02 | 9.47E−05 | 2.77E−08 |
| FLAG-BAP | Anti-FLAG-M2-V7 | 4.57E+05 | 9.47E+02 | 1.06E−02 | 1.76E−05 | 2.32E−08 |
| FLAG-BAP | BEAT 2a1 | 1.06E+06 | 5.36E+03 | 5.65E−03 | 1.99E−05 | 5.35E−09 |
| FLAG-BAP | BEAT 2a1 | 1.61E+06 | 1.39E+04 | 5.50E−03 | 4.04E−05 | 3.41E−09 |
| FLAG-BAP | BEAT 2a1 | 1.02E+06 | 5.95E+03 | 1.64E−02 | 7.20E−05 | 1.62E−08 |
| FLAG-BAP | BEAT 2a2 | 2.67E+05 | 6.80E+02 | 5.81E−03 | 6.28E−06 | 2.17E−08 |
| FLAG-BAP | BEAT 2a2 | 1.82E+05 | 1.74E+03 | 9.46E−03 | 8.80E−05 | 5.19E−08 |
| FLAG-BAP | BEAT 2a3 | 5.46E+06 | 8.72E+04 | 1.66E−02 | 2.66E−04 | 3.04E−09 |
| FLAG-BAP | BEAT 2a3 | 1.37E+06 | 9.13E+03 | 3.60E−03 | 9.81E−06 | 2.63E−09 |
| FLAG-BAP | BEAT 2a3 | 1.20E+06 | 7.56E+03 | 6.18E−03 | 3.14E−05 | 5.13E−09 |
| FLAG-BAP | BEAT 2a4 | 5.34E+05 | 3.41E+03 | 7.10E−03 | 2.96E−05 | 1.33E−08 |
| FLAG-BAP | BEAT 2a4 | 3.02E+05 | 5.68E+02 | 5.13E−03 | 9.17E−06 | 1.70E−08 |
| FLAG-BAP | BEAT 2a5 | 1.07E+06 | 4.82E+03 | 1.95E−02 | 5.02E−05 | 1.81E−08 |
| FLAG-BAP | BEAT 2a5 | 9.36E+05 | 3.19E+03 | 1.82E−02 | 3.49E−05 | 1.95E−08 |
| FLAG-BAP | BEAT 2a6 | 2.60E+05 | 5.20E+02 | 9.14E−03 | 1.47E−05 | 3.51E−08 |
| FLAG-BAP | BEAT 2a6 | 2.68E+05 | 4.22E+02 | 8.73E−03 | 1.09E−05 | 3.26E−08 |
| FLAG-BAP | BEAT 2a7 | 2.08E+06 | 1.66E+04 | 1.97E−02 | 7.39E−05 | 9.46E−09 |
| FLAG-BAP | BEAT 2a7 | 2.56E+06 | 5.49E+04 | 1.69E−02 | 3.68E−04 | 6.59E−09 |
| FLAG-BAP | BEAT 2a8 | 4.43E+05 | 4.34E+02 | 4.03E−03 | 2.51E−06 | 9.09E−09 |
| FLAG-BAP | BEAT 2a8 | 4.17E+05 | 4.76E+02 | 4.49E−03 | 2.83E−06 | 1.08E−08 |
| FLAG-BAP | BEAT 2b1 | 1.23E+06 | 2.36E+03 | 2.79E−03 | 3.36E−06 | 2.27E−09 |
| FLAG-BAP | BEAT 2b1 | 1.34E+06 | 2.30E+03 | 2.25E−03 | 2.84E−06 | 1.68E−09 |
| FLAG-BAP | BEAT 2b2 | 1.41E+05 | 6.54E+02 | 1.02E−02 | 2.17E−05 | 7.22E−08 |
| FLAG-BAP | BEAT 2b2 | 2.36E+05 | 7.14E+02 | 8.82E−03 | 2.30E−05 | 3.74E−08 |
| FLAG-BAP | BEAT 2b3 | 1.14E+06 | 9.09E+02 | 1.27E−03 | 1.16E−06 | 1.12E−09 |
| FLAG-BAP | BEAT 2b3 | 1.07E+06 | 7.35E+02 | 1.15E−03 | 9.74E−07 | 1.08E−09 |
| FLAG-BAP | BEAT 2b4 | 3.07E+05 | 5.38E+02 | 2.53E−03 | 3.98E−06 | 8.24E−09 |
| FLAG-BAP | BEAT 2b4 | 2.70E+05 | 3.83E+02 | 2.22E−03 | 2.29E−06 | 8.23E−09 |

### Example 14—Binding of BEAT2 to APCs

BEATs were evaluated to binding to APCs at 10, 30 and 100 nM. The IgG2 isotype control had no binding and the B11 anti-CD206 Mab control had the highest binding (100 nM only) (FIG. 6B). BEATs 2a6, 2a8 and 2b3 had the highest binding.

### Example 15—Functional Activity of BEAT2 on APCs and CAR T Cells

CART plus MoDCs were evaluated with the BEATs. MoDCs were confirmed to have CD206 expression (FIG. 9B). Binding via the FLAG tag stimulated IFN-γ response. Increases in IFN-γ were seen with the addition of the anti-CD206 BEATs (FIG. 9A). Negative controls (IgG1 and IgG2 isotypes) had no effect on IFN-γ. Lead anti-CD206 BEATs included 2b3.

### Example 16—BEAT2 Cytokine Analysis

A cytokine analysis was completed with one donor testing the lead BEAT2s. anti-CD206 BEAT2s stimulated IFN-γ, TNF-α, and IL-6 above the cells alone background (FIG. 10). There was limited activity with IL-2, IL-4, IL-10, IL-8, IP-10, MCP-1, IL-12p70 and IL-17A.

### Example 17—T Cell Proliferation with 7B1 28z CAR+BEAT2+MoDC

The proliferation of 7B1 28z CAR T cells in the presence of control compounds/BEAT2 (30 nM) and MoDCs was examined after 120 h proliferation.

Increase in fold change was observed relative to controls for CD3+, CD4+ and CD8+ cells (FIG. 11) for anti-CD206 BEATs 2B3 and 2B4, in particular there was an increase the percentage of central memory T cells with addition of the BEAT 2B4 to the CD8+ T cells (FIG. 12).

### Example 18—In Vivo Efficacy of CAR T+ anti-CD206 BEAT

BEAT2a6 was evaluated in an NSG mouse model using SKOV3-Mesothelin transfected CD40 knock-out cells. Tumours were first established (50 mm²) in the NSG mice. A single dose of 5 million anti-mesothelin CARs/5 million MoDCs (with high expression of mesothelin) and bi-weekly doses of BEAT at 12.5 ug BEAT (5 doses). (FIG. 14A).

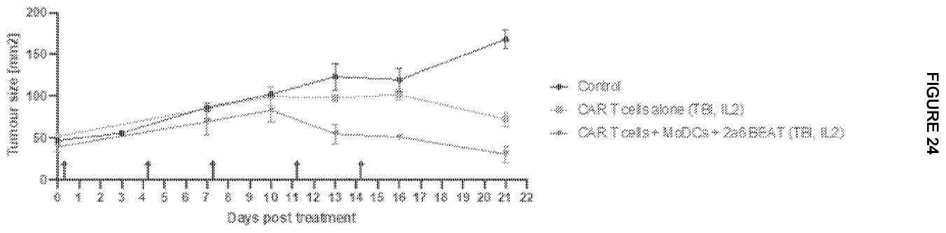

Tumour size was measured for CART cells+MoDCs+BEAT 2a6 compared to CAR T cells alone or no treatment control (FIG. 24).

### Example 19—One Arm BEATs Expression and Purification

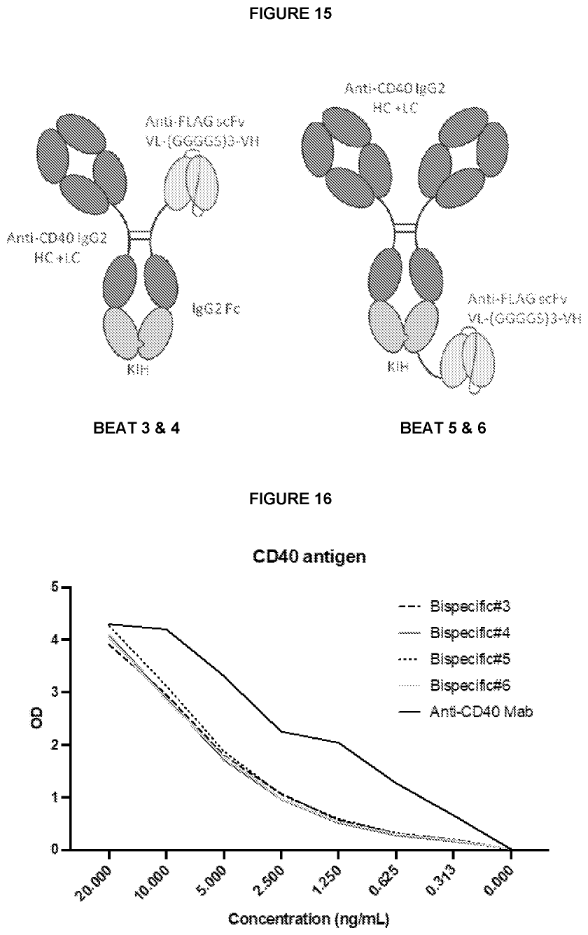

One arm anti-CD40 and anti-FLAG BEATS were designed using two different Knobs-in-Holes mutations to form bispecific BEAT3&4 and BEAT 5&6 (FIG. 15).

### BEAT3:

One arm with anti-CD40 IgG2 (Fab+Fc) with KIH mutation set #1
One arm with anti-FLAG scFv+Fc (hM2V4 scFv in the VL-(GGGGs)3-VH) with KIH mutation with KIH mutation set #1
   Heavy chain 1
      HC-IgG2(anti-CD40)KIH1a
   Light chain 1
      LC-IgG2(anti-CD40)
   Heavy chain 2
      HC-hM2V4_CurrusLinker-(GGGGS)3-IgG2 Fc-KIH1b

### BEAT4:

One arm with anti-CD40 IgG2 (Fab+Fc) with KIH mutation set #2
One arm with anti-FLAG scFv+Fc (hM2V4 scFv in the VL-(GGGGs)3-VH) with KIH mutation with KIH mutation set #2
   Heavy chain 1
      HC-IgG2(anti-CD40)KIH2a
   Light chain 1
      LC-IgG2(anti-CD40)
   Heavy chain 2
      HC-hM2V4_CurrusLinker-(GGGGS)3-IgG2 Fc-KIH2b

### BEAT5:

One arm bivalent with anti-CD40 IgG2 with coupled anti-FLAG scFv (at the C terminus of the heavy chain) with KIH mutation set #1
One arm with anti-CD40 IgG2 with KIH mutation set #1
   Heavy chain 1
      HC-IgG2(anti-CD40)KIH1a
   Light chain 1
      LC-IgG2(anti-CD40)
   Heavy chain 2
      HC-IgG2(anti-CD40)-(GGGGS)3-hM2V4_CurrusLinker-KIH1 b
   Light chain 2
      LC-IgG2(anti-CD40)

### BEAT6:

One arm bivalent with anti-CD40 IgG2 with coupled anti-FLAG scFv (at the C terminus of the heavy chain) with KIH mutation set #2
One arm with anti-CD40 IgG2 with KIH mutation set #2
   Heavy chain 1
      HC IgG2(anti-CD40)KIH2a
   Light chain 1
      LC-IgG2(anti-CD40)
   Heavy chain 2
      HC-IgG2(anti-CD40)-(GGGGS)3-hM2V4_CurrusLinker-KIH2b

Light chain 2
LC-IgG2(anti-CD40)

The cDNAs of variable heavy (VH) and variable light (VL) sequences corresponding to all 4 BEAT variants were synthesized and sub-cloned into a mammalian cell expression vector pTXs1.

500 mL XtenCHO cultures were transfected with one arm BEAT (BEAT 3, 4, 5, 6) encoding vectors. Cultures were harvested by centrifugation at 14 days post transfection. Clarified supernatants of the proteins were purified by Protein A Affinity Chromatography ProtA (Agarose Resin) & Buffer exchanged (PBS). Materials were dialysed into PBS buffer. 15 μg samples were formulated in 4× loading buffer (reducing and non-reducing) and analyzed by SDS-PAGE and SEC-HPLC.

More than 10 mgs of antibody was expressed for all BEAT formats with greater than 90% purity as shown in the below table:

| | BEAT 3 | | BEAT 4 | | BEAT 5 | | BEAT 6 | |
|---|---|---|---|---|---|---|---|---|
| Yield/purity | 100 mg/L | 69.45% | 106 mg/L | 67.95% | 104 mg/L | 69.67% | 101 mg/L | 49.27% |
| after | | | | | | | | |
| Protein A | | | | | | | | |
| Yield/purity | 26 mg/L | 92.06% | 26 mg/L | 94.95% | 40 mg/L | 99.66% | 20 mg/L | 92.28% |
| after SEC | | | | | | | | |

### Example 15—Affinity of One Arm BEATs to CD40 and FLAG-tag

Binding of one arm BEATs to CD40 was evaluated by ELISA. All BEATs had similar binding to CD40 (FIG. 16). The affinity of the one arm BEAT to CD40 or to FLAG was confirmed via SPR using a 1:1 model. The affinity of the one arm BEATs was equivalent to the anti-CD40 control Mab and BEAT1a4 using the 1:1 model as shown in the below table:

| | CD40 | FLAG |
|---|---|---|
| anti-CD40 control Mab | 0.77 nM | n/a |
| Beat1a4 | 0.59 nM | 2.59 nM |
| BEAT 3 | 0.52 nM | 0.475 nM |
| BEAT 4 | 0.92 nM | 0.357 nM |
| BEAT 5 | 0.73 nM | 1.95 nM |
| BEAT 6 | 0.42 nM | 1.44 nM |

### Example 16—Binding of One Arm BEATs to CAR T and AP Cells

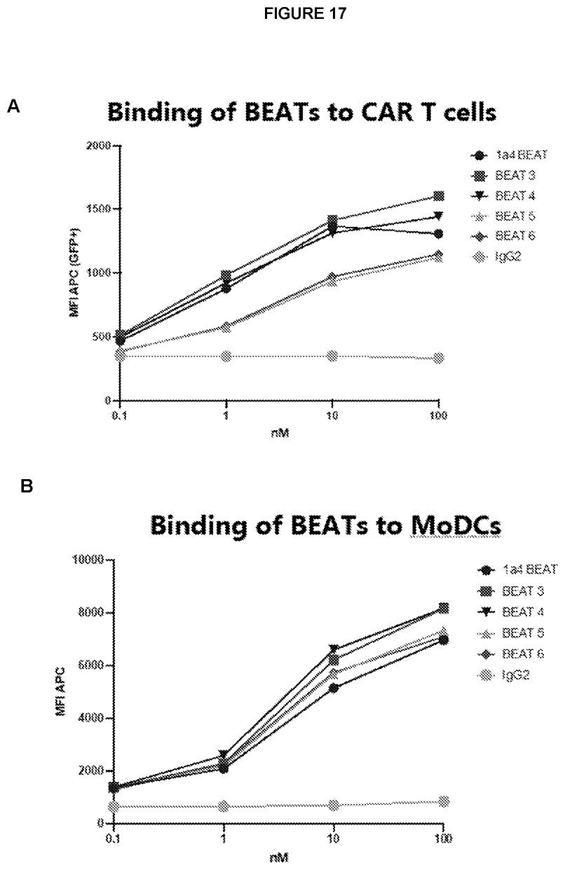

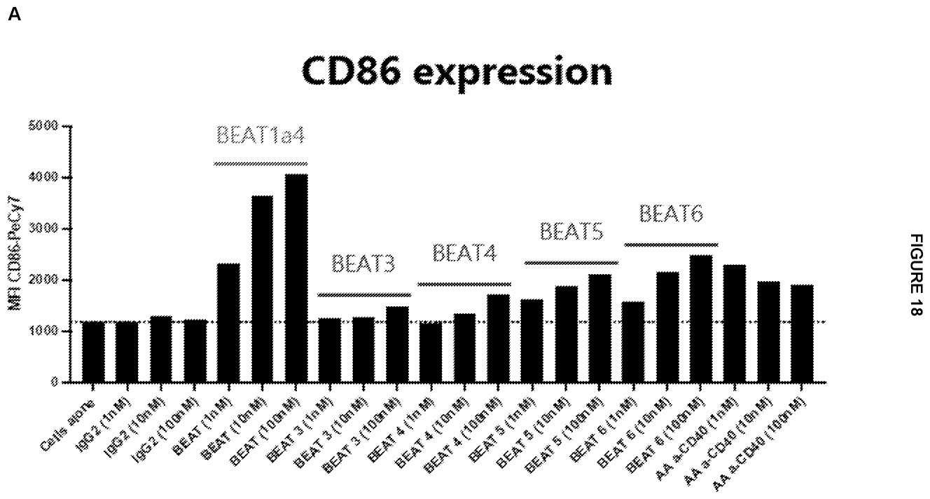

Binding of monovalent one arm BEAT 3 and 4 to CAR T cells was equivalent to bivalent BEAT1a4 with BEAT5 and 6 showing slightly decreased binding compared with BEAT 1a4, 3 and 4 (FIG. 17A). Binding of monovalent one arm BEATs to MoDCs was also equivalent to the bivalent BEAT1a4 (FIG. 17B).

### Example 17—Functional Activity of One Arm BEATs

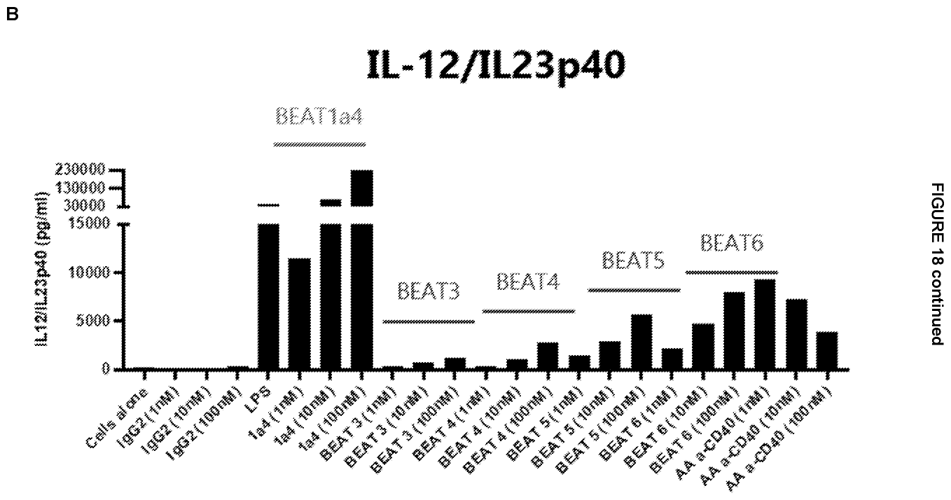

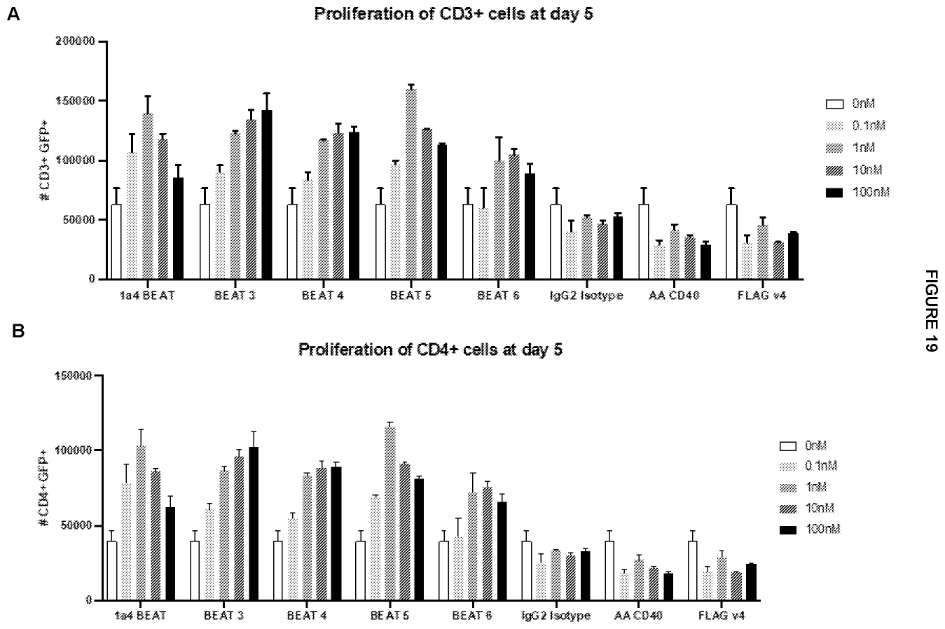

CD86 expression and IL-12/IL-23p40 release were evaluated in the presence of the BEATs and compared to BEAT1a4 and an anti-CD-40 control antibody. The one arm BEATs had a lower activity compared to BEAT1a4, with BEAT 3 and BEAT 4 (monovalent anti-CD40) decreased compared to BEAT 5 and BEAT 6 (bivalent anti-CD40) (FIG. 18) for both CD86 expression and IL-12/IL-23p40 release.

### Example 18—T-Cell Proliferation at Day 5 with One Arm BEATs

Proliferation of one arm BEATs was compared to BEAT1a4 in a dose response assay when added to both CAR T and MoDCs. 7B1-CD28z CARs were incubated with MoDCs and various BEATs and controls for 120 h and CAR T cell numbers were measured (via CD4+GFP+).

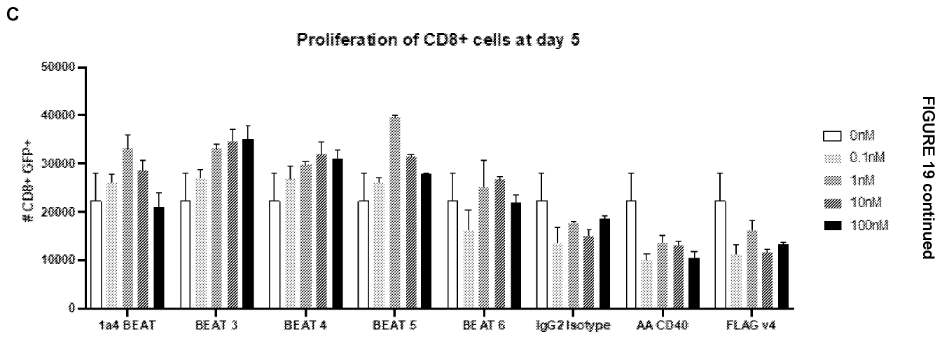

A prozone effect was seen with CAR T cell proliferation with BEAT1a4 at 1 nM. Dose responses were seen with various one arm BEATs with BEAT3 the most potent, followed by BEAT4, BEAT5 and BEAT6 at the highest dose with equivalent proliferation seen between 0.1-10 nM and a prozone effect seen with BEAT5 above 1 nM (FIG. 19).

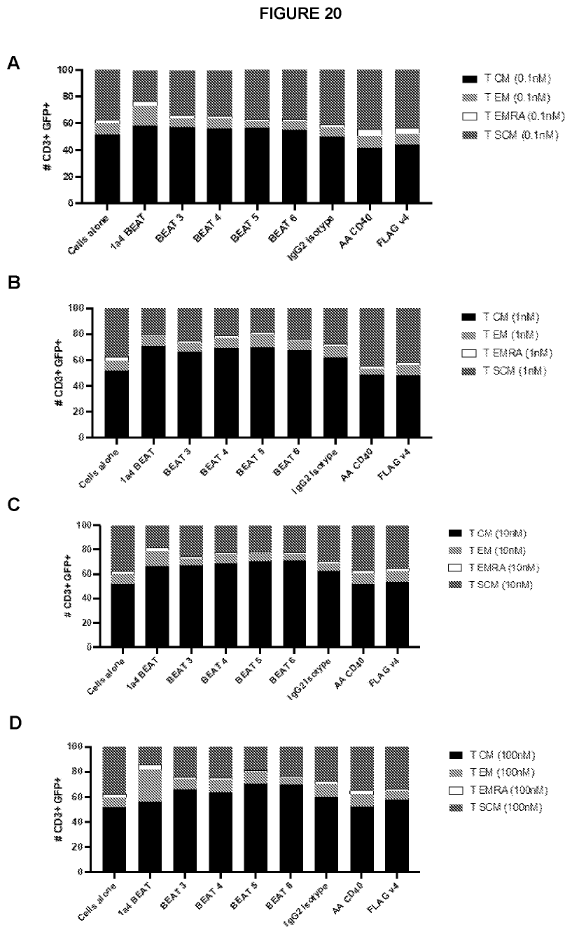

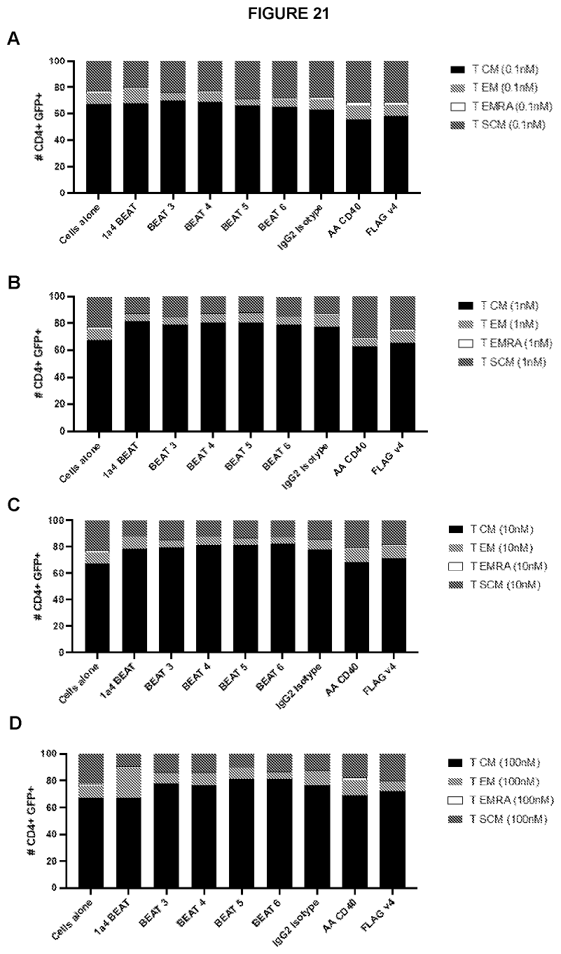

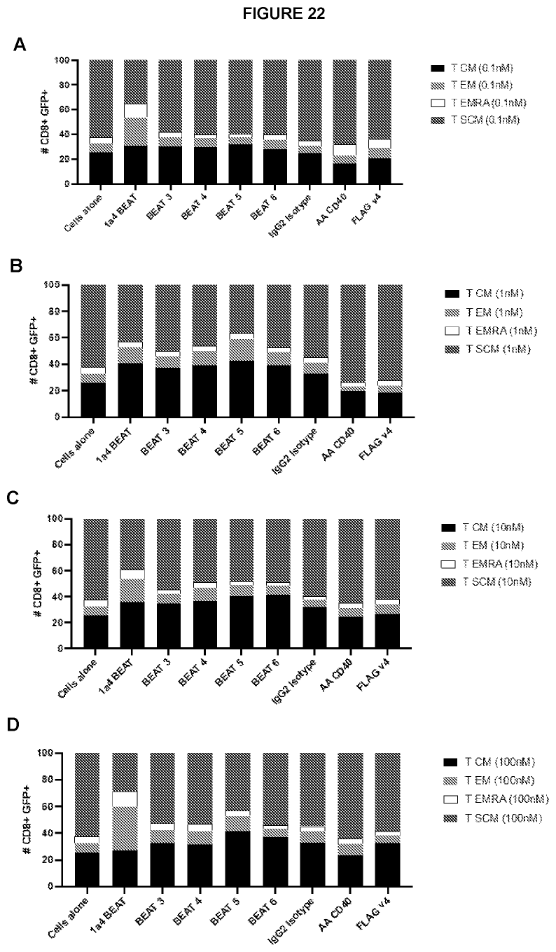

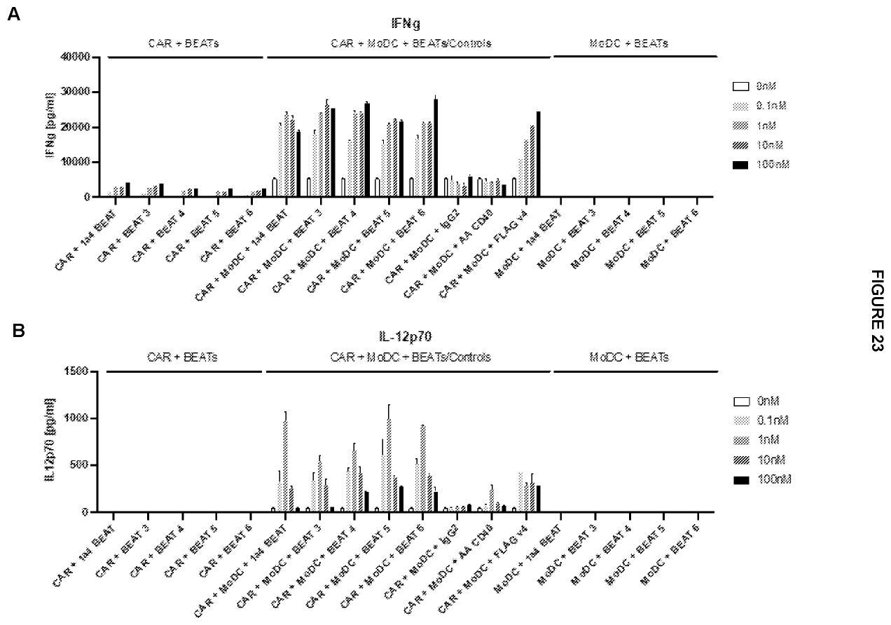

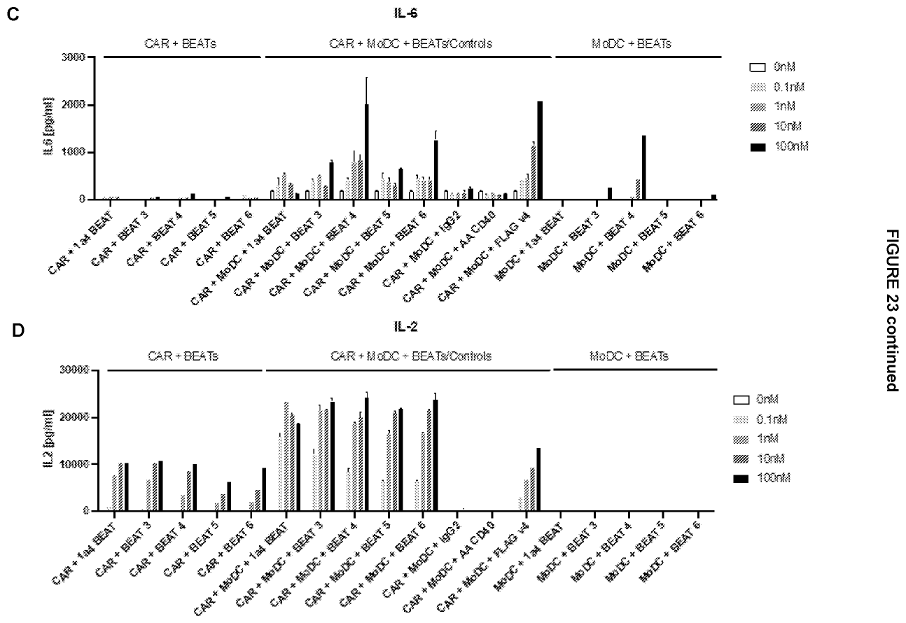

The phenotypes of cells were also evaluated in this proliferation assay, with increased TEM of T cells (FIGS. 20 A, C and D) and driven by increases mainly in the CD8+ cells (FIG. 22 A, B, C, D), with CD4+ cell with increased TEM at the highest dose of 100 nM dose (FIG. 21D). The phenotypes of T cells (FIG. 20), CD4+ cells (FIG. 21), and CD8+ cells (FIG. 22), with the one arm BEATs were similar with all doses.

### Example 19—Cytokine Analysis of One Arm BEATs

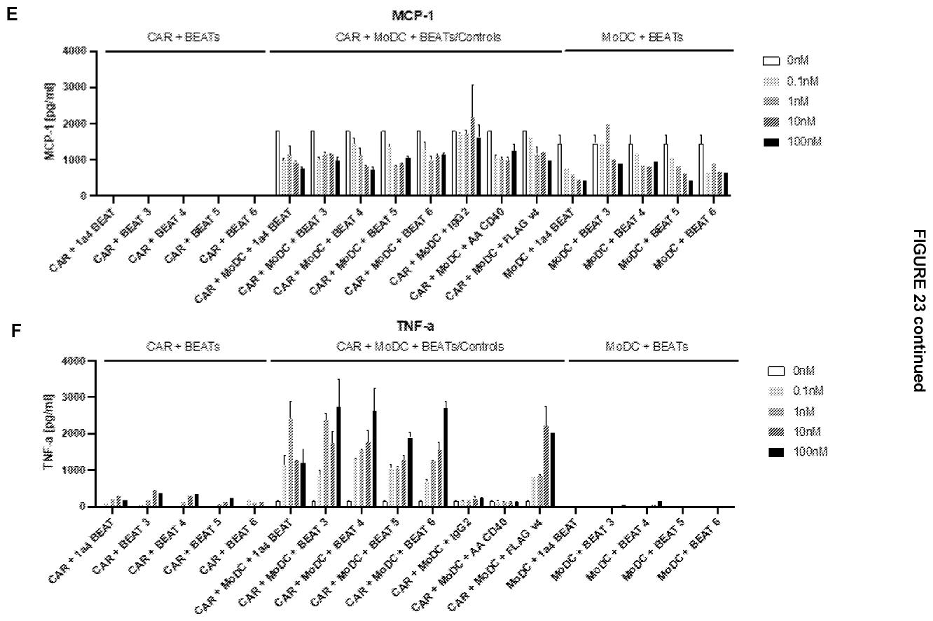

Cytokines were analysed in the presence of CARs, CAR+MoDCs and MoDCs alone. BEATs had no impact on IFNγ in the presence of CARs alone or MoDCs alone (FIG. 23A). IFNγ was increased in the presence of CAR T cells+MoDCs and BEATs in a dose responsive manner with equivalent responses seen with all BEATs and maximum responses seen at 1 nM (FIG. 23A).

BEATs had no impact on IL-12p70 or IL-6 in the presence of CARs alone or MoDCs alone (FIG. 23B and FIG. 23C). IL-12p70 increased at 0.1 nM and 1 nM with all BEATs and had a prozone effect above 1 nM in the presence of CARs and MoDCs (FIG. 23B). IL-6 increased with the addition of the BEATs to CARs and MoDCs in a dose response manner with BEAT 4 cytokine levels greater than other BEATs (FIG. 23C).

IL-2 increased in the presence of CARs alone and in a dose responsive manner in the presence of BEAT+CAR+MoDCs (FIG. 23D). No change was seen with MCP-1 above background (FIG. 23E). TNF-α increased in the presence of BEAT+CAR+MoDCs with no change with CAR or MoDCs alone (FIG. 23F).

1. A bispecific polypeptide comprising a first antigen binding protein and a second antigen binding protein, wherein the first antigen binding protein specifically binds to CD40 and wherein the second antigen binding protein specifically binds to a FLAG tag,
wherein the first antigen binding protein binds to or specifically binds to CD40 and wherein the first antigen binding protein comprises a CDRH1, a CDRH2 and/or a CDRH3 of an antigen binding domain having a variable heavy domain as defined in SEQ ID NO: 1; and/or a CDRL1, a CDRL2 and/or a CDRL3 of an antigen binding domain having a variable light chain as defined in of SEQ ID NO: 2.

2-6. (canceled)

7. The bispecific polypeptide of claim 1, wherein the first antigen binding protein comprises:
(i) a VH comprising a complementarity determining region (CDR) 1 comprising or consisting of a sequence as set forth in SEQ ID NO: 3, a CDR2 comprising or consisting of a sequence as set forth in SEQ ID NO: 6 and a CDR3 comprising or consisting of a sequence as set forth in SEQ ID NO: 10, and
(ii) a VL comprising a complementarity determining region (CDR) 1 comprising or consisting of a sequence as set forth in SEQ ID NO: 12, a CDR2 comprising or consisting of a sequence as set forth in SEQ ID NO: 14 and a CDR3 comprising or consisting of a sequence as set forth in SEQ ID NO: 16;
or
(iii) a VH comprising a complementarity determining region (CDR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 3, a CDR2 comprising or consisting of a sequence at least about 80%, at least 85%, at least 90%, at least 92%, at least 95%, at least 97%, at least 99% identical to a sequence set forth in SEQ ID NO: 6; a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 10, and
(iv) a VL comprising a CDR1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 12, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 14, a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 16;
or
(v) a VH comprising a complementarity determining region (CDR) 1 comprising or consisting of a sequence as set forth in SEQ ID NO: 5, a CDR2 comprising or consisting of a sequence as set forth in SEQ ID NO: 9 and a CDR3 comprising or consisting of a sequence as set forth in SEQ ID NO: 11, and
(vi) a VL comprising a complementarity determining region (CDR) 1 comprising or consisting of a sequence as set forth in SEQ ID NO: 13, a CDR2 comprising or consisting of a sequence as set forth in SEQ ID NO: 15 and a CDR3 comprising or consisting of a sequence as set forth in SEQ ID NO: 16;
or
(vii) a VH comprising a complementarity determining region (CDR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 5, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 9; a CDR3 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 11, and
(viii) a VL comprising a CDR1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 13, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 15, a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 16;
or
(ix) a VH comprising a complementarity determining region (CDR) 1 comprising or consisting of a sequence as set forth in SEQ ID NO: 4, a CDR2 comprising or consisting of a sequence as set forth in SEQ ID NO: 7 and a CDR3 comprising or consisting of a sequence as set forth in SEQ ID NO: 10, and
(x) a VL comprising a complementarity determining region (CDR) 1 comprising or consisting of a sequence as set forth in SEQ ID NO: 12, a CDR2 comprising or consisting of a sequence as set forth in SEQ ID NO: 14 and a CDR3 comprising or consisting of a sequence as set forth in SEQ ID NO: 16;
or
(xi) a VH comprising a complementarity determining region (CDR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 4, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 7; a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 10, and
(xii) a VL comprising a CDR1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 12, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 14, a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 16;
or
(xiii) a VH comprising a complementarity determining region (CDR) 1 comprising or consisting of a sequence as set forth in SEQ ID NO: 4, a CDR2 comprising or consisting of a sequence as set forth in SEQ ID NO: 8 and a CDR3 comprising or consisting of a sequence as set forth in SEQ ID NO: 10, and
xiv a VL comprising a complementarity determining region (CDR) 1 comprising or consisting of a sequence as set forth in SEQ ID NO: 12, a CDR2 comprising or consisting of a sequence as set forth in SEQ ID NO: 14 and a CDR3 comprising or consisting of a sequence as set forth in SEQ ID NO: 16;
or
(xv) a VH comprising a complementarity determining region (CDR) 1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 4, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 8; a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 10, and
(xvi) a VL comprising a CDR1 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 12, a CDR2 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 14, a CDR3 comprising or consisting of a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence set forth in SEQ ID NO: 16.

8. The bispecific polypeptide of claim 1, wherein:
(a) the first antigen binding protein comprises a heavy chain variable domain comprising or consisting of the amino acid sequence as set forth in SEQ ID NO: 1, or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto;
optionally wherein the heavy chain variable domain of the first antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEQ ID NO: 1; wherein the amino acid substitutions are not in the CDRs and/or the antigen binding protein retains the ability to bind to CD40;
and/or
(b) the first antigen binding protein comprises a light chain variable domain comprising or consisting of the amino acid sequence as set forth in SEQ ID NO: 2; or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto;
optionally wherein the light chain variable domain of the first antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEQ ID NO: 2; wherein the amino acid substitutions are not in the CDRs and/or the antigen binding protein retains the ability to bind to CD40.

9-23. (canceled)

24. The bispecific polypeptide of claim 1, wherein:
(a) the first antigen binding protein comprises from N to C terminus a VH then VL, or a VL then VH, or any CDR 1, 2 and 3 defined herein as VH then any CDR 1, 2 and 3 defined herein as VL, or any CDR 1, 2 and 3 defined herein as VL then any CDR 1, 2 and 3 defined herein as VH; and/or
(b) the first antigen binding protein is in the form of:
(i) a single domain antibody (sdAb);
(ii) a single chain Fv fragment (scFv);
(iii) a dimeric scFv (di-scFv); or
(iv) one of (ii) to (iii) linked to a constant region of an antibody, Fc or a heavy chain constant domain (CH) 2 and/or CH3; and/or
(c) the first antigen binding protein is in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody; or
(viii) one of (i) to (vii) linked to a constant region of an antibody, Fc or a heavy chain constant domain (CH) 2 and/or CH3.

25-26. (canceled)

27. The bispecific polypeptide of claim 1, wherein the first antigen binding protein is in the form of an immunoglobulin G molecule (IgG).

28. The bispecific polypeptide of claim 1, wherein:
(a) the second antigen binding protein is capable of specifically binding to a FLAG tag or variant or modified form thereof; and/or
(b) the second antigen binding protein is for binding to a FLAG tag as defined in SEQ ID NOs: 80 and 99 or a variant thereof, such as wherein the FLAG-tag that contains, comprises or consists of the sequence GDYKDDDDKG (SEQ ID NO: 98), DYKDDDDK (SEQ ID NO: 99), MDYKDDDDK (SEQ ID NO: 100), DFKDDDK (SEQ ID NO: 101), DYKAFDNL (SEQ ID NO: 102), DYKDHDG (SEQ ID NO: 103), MDFKDDDDK (SEQ ID NO: 104), MDYKAFDNL (SEQ ID NO: 105), DYKDHDI (SEQ ID NO: 106), DYKDH (SEQ ID NO: 107), DYKDD (SEQ ID NO: 108), DYKDHD (SEQ ID NO: 109) and/or DYKDDD (SEQ ID NO: 110); and/or
(c) the FLAG tag is present at the N-terminus, the C-terminus or within the protein to which the bispecific polypeptide is capable of binding to, binds to or specifically binds to.

29-31. (canceled)

32. The bispecific polypeptide of claim 1, wherein the second antigen binding protein comprises a CDRH1, a CDRH2 and/or a CDRH3 of an antigen binding domain having a variable heavy chain as defined in any one of SEQ ID NOs: 71 to 74, and/or a CDRL1, a CDRL2 and/or a CDRL3 of an antigen binding domain having a variable light chain as defined in any one of SEQ ID NOs: 76 to 79.

33. The bispecific polypeptide of claim 1, wherein the second antigen binding protein comprises an antigen binding domain comprising:
(a) a VH comprising a CDR1 comprising or consisting of an amino acid sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a sequence as set forth in SEQ ID NO: 81; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 82; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 83, and
(b) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 90; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 91; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 92;
or
(c) a VH comprising a complementarity determining region CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NOs: 111 or 112; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 113; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 114, and
(d) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 120; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 121; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 92;
or
(e) a VH comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NOs: 111 or 112; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 113; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 114, and
(f) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 133; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 121; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 92;
or
(g) a VH comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NOs: 111 or 156; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 157; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 114, and
(h) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 120; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 121; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 92;
or
(i) a VH comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NOs: 111 or 156; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 157; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 114, and
(j) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NOs: 133; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 121; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 92;
or
(k) a VH comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 126; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 127; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 114, and
(l) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 120; a CDR2 comprising or consisting of an amino acid sequence of at least about 80%, at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 121; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 92;
or
(m) a VH comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 126; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 127; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 114, and
(n) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 133; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 121; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 92;
or
(o) a VH comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 111; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 132; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 114, and
(p) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 133; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 121; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 92;
or
(q) a VH comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 126; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 127; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 114, and
(r) a VL comprising a CDR1 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 133; a CDR2 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 121; a CDR3 comprising or consisting of an amino acid sequence of at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the sequence of SEQ ID NO: 92.

34. The bispecific polypeptide of claim 33, wherein the second antigen binding protein comprises an antigen binding domain comprising:
(a) a VH comprising a CDR1 comprising or consisting of an amino acid sequence of SEQ ID NO: 81; a CDR2 comprising or consisting of an amino acid sequence of SEQ ID NO: 82; a CDR3 comprising or consisting of an amino acid sequence of SEQ ID NO: 83, and
(b) a VL comprising a CDR1 comprising or consisting of an amino acid sequence SEQ ID NO: 90; a CDR2 comprising or consisting of an amino acid sequence of SEQ ID NO: 91; a CDR3 comprising or consisting of an amino acid sequence SEQ ID NO: 92;
or
(c) a VH comprising a CDR1 comprising or consisting of an amino acid sequence of SEQ ID NOs: 111 or 112; a CDR2 comprising or consisting of an amino acid sequence of SEQ ID NO: 113; a CDR3 comprising or consisting of an amino acid sequence of SEQ ID NO: 114, and
(d) a VL comprising a CDR1 comprising or consisting of an amino acid sequence of SEQ ID NO: 120; a CDR2 comprising or consisting of an amino acid sequence of SEQ ID NO: 121; a CDR3 comprising or consisting of an amino acid sequence of SEQ ID NO: 92;
or
(e) a VH comprising a CDR1 comprising or consisting of an amino acid sequence of SEQ ID NOs: 111 or 112; a CDR2 comprising or consisting of an amino acid sequence of SEQ ID NO: 113; a CDR3 comprising or consisting of an amino acid sequence of SEQ ID NO: 114, and
(f) a VL comprising a CDR1 comprising or consisting of an amino acid sequence of SEQ ID NO: 133; a CDR2 comprising or consisting of an amino acid sequence SEQ ID NO: 121; a CDR3 comprising or consisting of an amino acid sequence of SEQ ID NO: 92;
or
(g) a VH comprising a CDR1 comprising or consisting of an amino acid sequence of SEQ ID NOs: 111 or 156; a CDR2 comprising or consisting of an amino acid sequence of SEQ ID NO: 157; a CDR3 comprising or consisting of an amino acid sequence of SEQ ID NO: 114, and
(h) a VL comprising a CDR1 comprising or consisting of an amino acid sequence of SEQ ID NO: 120; a CDR2 comprising or consisting of an amino acid sequence of SEQ ID NO: 121; a CDR3 comprising or consisting of an amino acid sequence of SEQ ID NO: 92;
or
(i) a VH comprising a CDR1 comprising or consisting of an amino acid sequence of SEQ ID NOs: 111 or 156; a CDR2 comprising or consisting of an amino acid sequence of SEQ ID NO: 157; a CDR3 comprising or consisting of an amino acid sequence of SEQ ID NO: 114, and
(j) a VL comprising a CDR1 comprising or consisting of an amino acid sequence of SEQ ID NO: 133; a CDR2 comprising or consisting of an amino acid sequence of SEQ ID NO: 121; a CDR3 comprising or consisting of an amino acid sequence of SEQ ID NO: 92;
or
(k) a VH comprising a CDR1 comprising or consisting of an amino acid sequence of SEQ ID NO: 126; a CDR2 comprising or consisting of an amino acid sequence of SEQ ID NO: 127; a CDR3 comprising or consisting of an amino acid sequence of SEQ ID NO: 114, and
(1) a VL comprising a CDR1 comprising or consisting of an amino acid sequence of SEQ ID NO: 120; a CDR2 comprising or consisting of an amino acid sequence of SEQ ID NO: 121; a CDR3 comprising or consisting of an amino acid sequence of SEQ ID NO: 92;
or
(m) a VH comprising a CDR1 comprising or consisting of an amino acid sequence of SEQ ID NO: 126; a CDR2 comprising or consisting of an amino acid sequence of SEQ ID NO: 127; a CDR3 comprising or consisting of an amino acid sequence of SEQ ID NO: 114, and
(n) a VL comprising a CDR1 comprising or consisting of an amino acid sequence of SEQ ID NO: 133; a CDR2 comprising or consisting of an amino acid sequence of SEQ ID NO: 121; a CDR3 comprising or consisting of an amino acid sequence of SEQ ID NO: 92;
or
(o) a VH comprising a CDR1 comprising or consisting of an amino acid sequence of SEQ ID NO: 111; a CDR2 comprising or consisting of an amino acid sequence of SEQ ID NO: 132; a CDR3 comprising or consisting of an amino acid sequence of SEQ ID NO: 114, and
(p) a VL comprising a CDR1 comprising or consisting of an amino acid sequence of SEQ ID NO: 133; a CDR2 comprising or consisting of an amino acid sequence of SEQ ID NO: 121; a CDR3 comprising or consisting of an amino acid sequence of SEQ ID NO: 92;
or
(q) a VH comprising a CDR1 comprising or consisting of an amino acid sequence of SEQ ID NO: 126; a CDR2 comprising or consisting of an amino acid sequence of SEQ ID NO: 127; a CDR3 comprising or consisting of an amino acid sequence of SEQ ID NO: 114, and
(r) a VL comprising a CDR1 comprising or consisting of an amino acid sequence of SEQ ID NO: 133; a CDR2 comprising or consisting of an amino acid sequence of SEQ ID NO: 121; a CDR3 comprising or consisting of an amino acid sequence of SEQ ID NO: 92.

35. The bispecific polypeptide of a m claim 1, wherein:
(a) the second antigen binding protein comprises an antigen binding domain with a FR H1, a FR H2, a FR H3 and/or a FR H4 from a human germline, wherein the human germline is IGHV1-46*01 or IGHV7-4-1*02; and/or wherein the second antigen binding protein comprises an antigen binding domain with a FR L1, a FR L2, a FR L3 and/or a FR L4 from a human germline, wherein the human germline is IGKV2-30*01 or IGKV4-1*01; and/or
(b) the second antigen binding protein comprises an antigen binding domain with a FR H1, a FR H2, a FR H3 and/or a FR H4 of an antigen binding domain having a variable heavy chain as defined in any one of SEQ ID NOs: 71 to 74, or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto;
optionally wherein the heavy chain variable domain of the second antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEQ ID NOs: 71 to 74; wherein the amino acid substitutions are not in the CDRs and/or the antigen binding protein retains the ability to bind to FLAG tag; and/or
(c) the second antigen binding protein comprises an antigen binding domain with a FR L1, a FR L2, a FR L3 and/or a FR L4 of an antigen binding domain having a variable light chain as defined in any one of SEQ ID NOs: 76 to 79, or a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical thereto;
optionally wherein the light chain variable domain of the second antigen binding protein comprises no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19 or no more than 20 amino acid residue substitutions, compared to the amino acid sequence as set forth in SEO ID NOs: 76 to 79; wherein the amino acid substitutions are not in the CDRs and/or the antigen binding protein retains the ability to bind to FLAG tag.

36-37. (canceled)

38. The bispecific polypeptide of claim 33, wherein:
(I) the second antigen binding protein comprises an antigen binding domain, wherein the antigen binding domain comprises a VH comprising a complementarity determining region (CDR) 1, CDR2 and CDR3 as defined in (a) and a VL comprising a CDR1, CDR2 and CDR3 as defined in (b), and:
A. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 134, 135, 136 and 137 respectively, and for VL FRs the reference sequences are set forth in SEQ ID NO: 138, 139, 140 and 97 respectively;
or
B. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 84, 85, 87 and 89 respectively, and for VL FRs the reference sequences are set forth in SEQ ID NO: 138, 139, 140 and 97 respectively;
or
C. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 84, 85, 87 and 89 respectively, and for VL FRs the reference sequences are set forth in SEQ ID NO: 153, 154, 155 and 97 respectively;
or
D. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 84, 85, 87 and 89 respectively, and for VL FRs the reference sequences are set forth in SEQ ID NO: 93, 95, 96 and 97 respectively;
or
E. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 85, 87 and 89 respectively, and for VL FRs the reference sequences are set forth in SEQ ID NO: 93, 94, 96 and 97 respectively;
or
F. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 86, 88 and 89 respectively, and for VL FRs the reference sequences are set forth in SEQ ID NO: 138, 139, 140 and 97 respectively;
or
G. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 84, 86, 88 and 89 respectively, and for VL FRs the reference sequences are set forth in SEQ ID NO: 153, 154, 155 and 97 respectively;
or
H. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 86, 88 and 89 respectively, and for VL FRs the reference sequences are set forth in SEQ ID NO: 93, 95, 96 and 97 respectively;
or
I. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 84, 86, 88 and 89 respectively, and for VL FRs the reference sequences are set forth in SEQ ID NO: 93, 94, 96 and 97 respectively;
or
J. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 149, 85, 151 and 89 respectively, and for VL FRs the reference sequences are set forth in SEQ ID NO: 138, 139, 140 and 97 respectively;
or
K. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 149, 85, 151 and 89 respectively, and for VL FRs the reference sequences are set forth in SEQ ID NO: 153, 154, 155 and 97 respectively;
or
L. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 149, 85, 151 and 89 respectively, and for VL FRs the reference sequences are set forth in SEQ ID NO: 93, 95, 96 and 97 respectively;
or
M. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 149, 85, 151 and 89 respectively, and for VL FRs the reference sequences are set forth in SEQ ID NO: 93, 94, 96 and 97 respectively;
or
N. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 149, 150, 152 and 89 respectively, and for VL FRs the reference sequences are set forth in SEQ ID NO: 138, 139, 140 and 97 respectively;
or
O. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 149, 150, 152 and 89 respectively, and for VL FRs the reference sequences are set forth in SEQ ID NO: 153, 154, 155 and 97 respectively;
or
P. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 149, 150, 152 and 89 respectively, and for VL FRs the reference sequences are set forth in SEQ ID NO: 93, 95, 96 and 97 respectively;
or
Q. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 149, 150, 152 and 89 respectively, and for VL FRs the reference sequences are set forth in SEQ ID NO: 93, 94, 96 and 97 respectively;
OR
(II) the second antigen binding protein comprises an antigen binding domain, wherein the antigen binding domain comprises a VH comprising a complementarity determining region (CDR) 1, CDR2 and CDR3 as defined in (c) and a VL comprising a CDR1, CDR2 and CDR3 as defined in (d), and:
A. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NO: 115, 116, 118 and 89 respectively, and for VL FRs the reference sequences are set forth in SEQ ID NO: 122, 124, 125 and 127 respectively;
or
B. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NO: 115, 116, 118 and 89 respectively, and for VL FRs the reference sequences are set forth in SEQ ID NO: 122, 123, 125 and 127 respectively;
or
C. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEO ID NO: 115, 117, 119 and 89 respectively, and for VL FRs the reference sequences are set forth in SEQ ID NO: 122, 124, 125 and 127 respectively;
or
D. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NO: 115, 117, 119 and 89 respectively, and for VL FRs the reference sequences are set forth in SEQ ID NO: 122, 123, 125 and 127 respectively;
OR
(III) the second antigen binding protein comprises an antigen binding domain, wherein the antigen binding domain comprises a VH comprising a complementarity determining region (CDR) 1, CDR2 and CDR3 as defined in (e) and a VL comprising a CDR1, CDR2 and CDR3 as defined in (f), and:
A. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 115, 116, 118 and 89 respectively, and for VL FRs the reference sequences are set forth in SEQ ID NO: 144, 145, 146 and 97 respectively;
or
B. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 115, 116, 118 and 89 respectively, and for VL FRs the reference sequences are set forth in SEQ ID NO: 161, 162, 163 and 97 respectively;
or
C. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 115, 117, 119 and 89 respectively, and for VL FRs the reference sequences are set forth in SEQ ID NO: 144, 145, 146 and 97 respectively;
or
D. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 115, 117, 119 and 89 respectively, and for VL FRs the reference sequences are set forth in SEQ ID NO: 161, 162, 163 and 97 respectively;
OR
(IV) the second antigen binding protein comprises an antigen binding domain comprising VH comprising a complementarity determining region (CDR) 1, CDR2 and CDR3 as defined in (g) and a VL comprising a CDR1, CDR2 and CDR3 as defined in (h), and:
A. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 158, 116, 159 and 89 respectively, and for VL FRs the reference sequences are set forth in SEQ ID NO: 122, 124, 125 and 127 respectively;
or
B. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 158, 116, 159 and 89 respectively, and for VL FRs the reference sequences are set forth in SEQ ID NO: 122, 123, 125 and 127 respectively;
or
C. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 158, 117, 160 and 89 respectively, and for VL FRs the reference sequences are set forth in SEQ ID NO: 122, 124, 125 and 127 respectively;
or
D. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 158, 117, 160 and 89 respectively, and for VL FRs the reference sequences are set forth in SEQ ID NO: 122, 123, 125 and 127 respectively;
OR
(V) the second antigen binding protein comprises an antigen binding domain, wherein the antigen binding domain comprises a VH comprising a complementarity determining region (CDR) 1, CDR2 and CDR3 as defined in (i) and a VL comprising a CDR1, CDR2 and CDR3 as defined in (j), and:
A. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 158, 116, 159 and 89 respectively, and for VL FRs the reference sequences are set forth in SEQ ID NO: 144, 145, 146 and 97 respectively;
or
B. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 158, 116, 159 and 89 respectively, and for VL FRs the reference sequences are set forth in SEQ ID NO: 161, 162, 163 and 97 respectively;
or
C. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 158, 117, 160 and 89 respectively, and for VL FRs the reference sequences are set forth in SEQ ID NO: 144, 145, 146 and 97 respectively;
or
D. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 158, 117, 160 and 89 respectively, and for VL FRs the reference sequences are set forth in SEQ ID NO: 161, 162, 163 and 97 respectively;
OR
(VI) the second antigen binding protein comprises an antigen binding domain, wherein the antigen binding domain comprises a VH comprising a complementarity determining region (CDR) 1, CDR2 and CDR3 as defined in (k) and a VL comprising a CDR1, CDR2 and CDR3 as defined in (1), and:
A. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 128, 130 and 89 respectively, and for VL FRs the reference sequences are set forth in SEQ ID NO: 122, 124, 125 and 97 respectively;
or
B. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 128, 130 and 89 respectively, and for VL FRs the reference sequences are set forth in SEQ ID NO: 122, 123, 125 and 97 respectively;
or
C. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 129, 131 and 89 respectively, and for VL FRs the reference sequences are set forth in SEQ ID NO: 122, 124, 125 and 97 respectively;
or
D. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 129, 131 and 89 respectively, and for VL FRs the reference sequences are set forth in SEQ ID NO: 122, 123, 125 and 97 respectively;
or
E. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical or is 100% identical to reference sequences: for VH FRs the reference sequences are set forth in SEQ ID NO: 164, 128, 166 and 89 respectively, and for VL FRs the reference sequences are set forth in SEQ ID NO: 122, 124, 125 and 97 respectively;
or
F. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 164, 128, 166 and 89 respectively, and for VL FRs the reference sequences are set forth in SEQ ID NO: 122, 123, 125 and 97 respectively;
or
G. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 164, 165, 167 and 89 respectively, and for VL FRs the reference sequences are set forth in SEQ ID NO: 122, 124, 125 and 97 respectively;
or
H. a VH and a VL each comprising framework regions (FR) 1, 2, 3 and 4 comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to reference sequences: for VH FRs the reference sequences are set forth in SEQ ID NO: 164, 165, 167 and 89 respectively, and for VL FRs the reference sequences are set forth in SEQ ID NO: 122, 123, 125 and 97 respectively;
OR
(VII) the second antigen binding protein comprises an antigen binding domain, wherein the antigen binding domain comprises a VH comprising a complementarity determining region (CDR) 1, CDR2 and CDR3 as defined in (m) and a VL comprising a CDR1, CDR2 and CDR3 as defined in (n), and:
A. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 128, 130 and 89 respectively, and for VL FRs the reference sequences are set forth in SEQ ID NO: 144, 145, 146 and 97 respectively;
or
B. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 128, 130 and 89 respectively, and for VL FRs the reference sequences are set forth in SEQ ID NO: 168, 169, 170 and 97 respectively;
or
C. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 129, 131 and 89 respectively, and for VL FRs the reference sequences are set forth in SEQ ID NO: 144, 145, 146 and 97 respectively;
or
D. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 129, 131 and 89 respectively, and for VL FRs the reference sequences are set forth in SEQ ID NO: 168, 169, 170 and 97 respectively;
or
E. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 164, 128, 166 and 89 respectively, and for VL FRs the reference sequences are set forth in SEQ ID NO: 144, 145, 146 and 97 respectively;
or
F. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 164, 128, 166 and 89 respectively, and for VL FRs the reference sequences are set forth in SEQ ID NO: 168, 169, 170 and 97 respectively;
or
G. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 164, 165, 167 and 89 respectively, and for VL FRs the reference sequences are set forth in SEQ ID NO: 144, 145, 146 and 97 respectively;
or
H. a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 164, 165, 167 and 89 respectively, and for VL FRs the reference sequences are set forth in SEQ ID NO: 168, 169, 170 and 97 respectively;
OR
(VIII) the second an antigen binding protein comprises an antigen binding domain, wherein the antigen binding domain comprises a VH comprising a complementarity determining region (CDR) 1, CDR2 and CDR3 as defined in (o) and a VL comprising a CDR1, CDR2 and CDR3 as defined in (p), and a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 141, 142, 143 and 137 respectively, and for VL FRs the reference sequences are set forth in SEQ ID NO: 144, 145, 146 and 97 respectively;
OR
(IX) the second an antigen binding protein comprises an antigen binding domain, wherein the antigen binding domain comprises a VH comprising a complementarity determining region (CDR) 1, CDR2 and CDR3 as defined in (q) and a VL comprising a CDR1, CDR2 and CDR3 as defined in (r) and a VH and a VL each comprising framework regions (FR) 1, 2, 3 and 4 comprising a sequence at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical to reference sequences: for VH FRs the reference sequences are set forth in SEQ ID NO: 84, 147, 148 and 137 respectively, and for VL FRs the reference sequences are set forth in SEQ ID NO: 144, 145, 146 and 97 respectively.

39-49. (canceled)

50. The bispecific polypeptide of claim 1, wherein:
(a) the second antigen binding protein is in the form of:
(i) a single domain antibody (sdAb);
(ii) a single chain Fv fragment (scFv);
(iii) a dimeric scFv (di-scFv); or
(iv) one of (ii) to (iii) linked to a constant region of an antibody, Fc or a heavy chain constant domain (CH) 2 and/or CH3; and/or
(b) the second antigen binding protein is in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody; or
(viii) one of (i) to (vii) linked to a constant region of an antibody, Fc or a heavy chain constant domain (CH) 2 and/or CH3; and/or
(c) the second antigen binding protein is in the form of an scFv and the first antigen binding protein is in the form of an immunoglobulin G (IgG) antibody; and/or
(d) the second antigen binding protein and the first antigen binding protein are both antibodies or antigen binding fragments thereof; optionally wherein the first and second antigen binding proteins are of the same antibody format, or fragment thereof; and/or
(e) the polypeptide is a fusion protein comprising the first and second antigen binding proteins.

51-53. (canceled)

54. The bispecific polypeptide of claim 1, wherein:
(A) the first antigen binding protein comprises, consists essentially of or consists of the amino acid sequence of (in order of N to C terminus or C to N terminus) SEQ ID NO: 1 and 2; and/or
(B) the second antigen binding domain comprises, consists essentially of or consists of an amino acid sequence of (in order of N to C terminus or C to N terminus);
(a) SEQ ID NO: 71 and 79;
(b) SEQ ID NO: 71 and 78;
(c) SEQ ID NO: 71 and 76;
(d) SEQ ID NO: 71 and 77;
(e) SEQ ID NO: 72 and 76;
(f) SEQ ID NO: 72 and 77;
(g) SEQ ID NO: 72 and 78;
(h) SEQ ID NO: 72 and 79;
(i) SEQ ID NO: 73 and 76;
(j) SEQ ID NO: 73 and 77;
(k) SEQ ID NO: 73 and 78;
(l) SEQ ID NO: 73 and 79;
(m) SEQ ID NO: 74 and 76;
(n) SEQ ID NO: 74 and 77;
(o) SEQ ID NO: 74 and 78; or
(p) SEQ ID NO: 74 and 79.

55. The bispecific polypeptide of claim 1, wherein the bispecific polypeptide comprises the amino acid sequences of:
SEQ ID NO: 19 and 20;
SEQ ID NO: 19 and 21;
SEQ ID NO: 22 and 23;
SEQ ID NO: 23 and 24;
SEQ ID NO: 19 and 25;
SEQ ID NO: 19 and 26;
SEQ ID NO: 23 and 27;
SEQ ID NO: 23 and 28;
SEQ ID NO: 19 and 29;
SEQ ID NO: 19 and 30;
SEQ ID NO: 23 and 31;
SEQ ID NO: 23 and 32;
SEQ ID NO: 171, 172 and 173;
SEQ ID NO: 172, 174 and 175;
SEQ ID NO: 171, 172 and 176; or
SEQ ID NO: 172, 174 and 177.

56. (canceled)

57. A fusion protein comprising the bispecific polypeptide of claim 1.

58. A nucleic acid encoding a bispecific polypeptide of claim 1.

59. (canceled)

60. A cell comprising a nucleic acid of claim 58.

61. A pharmaceutical composition comprising a bispecific polypeptide of claim 1.

62-63. (canceled)

64. A method of treating or preventing cancer, the method comprising administering to a subject a bispecific polypeptide of claim 1 thereby treating or preventing cancer.

65. A method of treating or preventing cancer, the method comprising administering to a subject a fusion protein comprising the bispecific polypeptide of claim 1, thereby treating or preventing cancer.
